(19) United States
(12) Patent Application Publication
(10) Pub. No.: US 2006/0018915 A1
(43) Pub. Date: Jan. 26, 2006

(54) Heteroclitic analogs and related methods

(76) Inventors: Glenn Ishioka, San Diego, CA (US); John Fikes, San Diego, CA (US); Shabnam Tangri, San Diego, CA (US); Alessandro Sette, La Jolla, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/510,101

(22) PCT Filed: Apr. 7, 2003
(86) PCT No.: PCT/US03/10571

§ 371 Date: Jul. 13, 2005

Related U.S. Application Data
(63) Continuation-in-part of application No. 10/116,118, filed on Apr. 5, 2002.
(60) Provisional application No. 60/413,471, filed on Sep. 26, 2002.

(51) Int. Cl. A61K 39/00; C07H 21/04; C12P 21/06; C07K 14/72

(52) U.S. Cl. 424/184.1; 435/69.1; 435/320.1; 435/325; 530/350; 536/23.5

(57) ABSTRACT

Heteroclitic analogs of class I epitopes are prepared by providing conservative, semi-conservative, or non-conservative amino acid substitutions at positions 3 and/or 4 and/or 5 and/or 6 and/or 7 and/or 8 and/or 9 and/or 10 of these epitopes. The analogs are useful in eliciting immune responses with respect to the corresponding wild-type epitopes.

### BACKGROUND OF THE INVENTION

1. Field of the Invention

The invention relates to methods for generating heteroclitic analogs of an original peptide which have increased stimulatory capacity for a given T cell.

2. Related Art

Several studies suggest that cytotoxic T lymphocytes (CTLs) play a central role in the eradication of infectious disease and cancer by the immune system (Byrne, et al., J. Immunol. 51:682 (1984), McMichael, et al., N. England J. Med., 309:13 (1983)). Since CTLs are stimulated by peptides comprising epitopes, considerable effort is ongoing in developing epitope-based vaccines that stimulate CTL responses. One class of epitopes, designated heteroclitic analogs, provides benefit as vaccine components since these analogs induce T cell responses stronger than those induced by the native epitope. Heteroclitic analogs are defined as peptides having increased stimulatory capacity or potency for a specific T cell, as measured by increased responses to a given dose, or by a requirement of lesser amounts to achieve the same response.

The advantages associated with using heteroclitic analogs in clinical applications are as follows. First, heteroclitic analogs have the ability to break/overcome tolerance by reversing a state of T cell anergy, activating non-tolerized cross-reactive clones of T cells, or by mediating “immune deviation,” i.e., the type of CTL produced, such as Th1 or Th2. Recent studies indicate that heteroclitic analogs are immunogenic (Zaremba, et al., Cancer Research, 57:4570 (1997); Rivoltoni, et al., Cancer Research, 59:301 (1999); Selby, et al., 162(2):669 (1999)) in that they are capable of inducing CTLs that recognize endogenously processed epitope. This is confirmed by studies in different immunological systems (Zugel, et al., J. Immunol., 161:1705 (1998), Wang, et al., J. Exp. Med., 190:983 (1999), Men, et al., J. Immunol., 162:3566, (1999)). For example, studies by Zugel et al. (Zugel, et al., supra) have shown that T cell tolerance to an immunodominant T cell epitope in adult mice can be overcome by immunization with heteroclitic cross-reactive peptide analogs of that peptide.

This is particularly significant in the field of cancer vaccines, where most of the CTL epitopes are derived from self-antigens. Due to the fact that cancer related antigens are often self-antigens, there is a corresponding phenomenon that there may be preexisting tolerance to these antigens, whereby generation of a T cell response to such epitopes is a challenge. Breaking of tolerance by heteroclitic analogs has been shown in a recent study in a murine class II system (Wang, et al., J. Exp. Med. 190:983 (1999)). In this study, the mechanism involved in breaking of tolerance was the stimulation of nontolerized, low affinity clones, rather than reversal of anergy. The heteroclicity demonstrated herein is associated with the induction of high avidity CTL, this represents an important difference.

Second, peptide analogs have been demonstrated to modulate cytokine production from T cells (Pfeiffer, et al., J. Exp. Med., 181:1569 (1995), Tao, et al., J. Immunol., 158:4237 (1997), Salazar, et al., Int. J. Cancer 85(6):829-38 (2000), Nicholson, et al., Int. Immunol. 12(2):205-13 (2000)). The immune deviation induced by such analogs has implications in several disease states, where generation of a specific subset of Th cell responses correlate with tumor regression (Zitvogel, et al., J. Exp. Med., 183:87 (1996), Celluzzi, et al., J. Exp. Med. 183:283 (1996)) or affect the clinical outcome of autoimmune or infectious disease (Romagnani, et al., Annu. Rev. Immunol., 12:227-57 (1994)). Thus, immunization with heteroclitic analogs offers the capacity to modulate cytokine production by induction of specific subsets of effector T cells, thereby altering the course of disease.

Third, heteroclitic analogs offer an advantage in drug development since significantly smaller amounts of peptide are needed for treatment doses, due to their strong biological potency. This feature overcomes certain manufacturing and toxicity concerns. In this regard, it has been shown that a heteroclitic analog of a MART-1 peptide (Rivoltini, et al., Cancer Research 59:301 (1999)), which generated antigen specific T cells in melanoma patients, was active at much lower concentrations than the native epitope. Similar results were reported by Schlom and colleagues (Zaremba, et al., Cancer Research 57:4570 (1997)) regarding a heteroclitic analog of the CEA-derived CAP1 epitope. However, a side-by-side precursor frequency analysis or a TCR avidity analysis against wild-type peptide was not performed.

Two types of well characterized cancer antigens are the carcinoembryonic antigen (CEA) and the melanoma antigens (MAGE). CEA is a 180 kDa cell surface and secreted glycoprotein overexpressed on most human adenocarcinomas including colon, rectal, pancreatic and gastric (Muraro et al., Cancer Res. 45:5769-5780, 1985) as well as 50% of breast (Steward et al., Cancer (Phila) 33:1246-1252, 1974) and 70% of non-small cell lung carcinomas (Vincent et al., J. Thorac. Cardiovasc. Surg. 66:320-328, 1978). CEA is also expressed, to some extent, on normal epithelium and in some fetal tissues (Thompson et al., J. Clin. Lab. Anal. 5:344-366, 1991). The abnormally high expression on cancer cells makes CEA an important target for immunotherapy.

MAGE are a family of related proteins that were first described in 1991. Van der Bruggen and co-workers identified the MAGE gene after isolating CTLs from a patient who demonstrated spontaneous tumor regression. These CTLs recognized melanoma cell lines as well as tumor lines from other patients all of whom expressed the same HLA-A1-restricted gene (van der Bruggen et al., Science 254:1643-1647, 1991; DePlaen et al., Immunogenetics 40:360-369, 1994). The MAGE genes are expressed in metastatic melanomas (see, e.g., Brasseur et al., Int. J. Cancer 63:375-380, 1995), non-small cell lung (Weynants et al., Int. J. Cancer 56:826-829, 1994), gastric (Inoue et al., Gastroenterology 109:1522-1525, 1995), hepatocellular (Chen et al., Liver 19:110-114, 1999), renal (Yamanaka et al., Human Pathol. 24:1127-1134, 1998), colorectal (Mori et al., Ann. Surg. 224:183-188, 1996), and esophageal (Quillien et al., Anticancer Res. 17:387-391, 1997) carcinomas as well as tumors of the head and neck (Lett et al., Acta Otolaryngol. 116:633-639, 1996), ovaries (Gillespie et al., Br J. Cancer 78:816-821, 1998; Yamada et al., Int. J. Cancer 64:388-393, 1995), bladder, and osteosarcoma (Sudo et al., J. Orthop. Res. 15:128-132, 1997). Thus, MAGE, including MAGE2/3, are important targets for cancer immunotherapy.

Accordingly, because of their biological relevance, it would be extremely useful to predict and/or identify amino acid substitutions that render heteroclitic activity to a given epitope such as CEA epitopes, MAGE epitopes and other epitopes, and to be able to predict other substitutions that will result in such heteroclitic activity. However, prior to the present disclosure there has been no easy method for predicting such substitutions for e.g. A3 and A24 epitopes or A2 and B7 epitopes. Indeed, in previous studies (Selby, et al., J. Immunol., 162(2):669 (1999), Skipper, et al., J. Exp. Med. 183:527 (1996)), heteroclitic epitopes were fortuitously identified by eluting naturally occurring mutant peptides from melanoma cells, or by systematically screening a large number of analogs consisting of substitutions at almost every position in the epitope (Zaremba, et al., Cancer Research, 57:4570 (1997), Loftus, et al., Cancer Research 58:2433 (1998), Blake, et al., J. Exp. Med. 18:121 (1996)). Alternatively, heteroclitic analogs were identified by screening random combinatorial peptide libraries which also has required the arduous synthesis and screening of large numbers of peptides (Pinilla, et al., Current Opinion in Immunology 11:193-202 (1999)). Genetic approaches, such as screening of DNA expression libraries, have provided another method for generating CTL epitopes and analogs (Boon, et al., Annu. Rev. Immunol. 12:337-65 (1994), Gavin, et al., Eur. J. Immunol. 24(9):2124-33 (1994)). However, these approaches may be problematic given the potentially small quantities and complexity of epitopes generated.

### BRIEF SUMMARY OF THE INVENTION

The invention provides methods to prepare peptides containing epitopes which have enhanced ability to effect an immune response with respect to corresponding wild-type epitopes. The resulting “heteroclitic analogs” are useful in immunological compositions for treatment of viral diseases, cancer, and other conditions which are characterized by displayed antigens on target cells.

The present inventors developed heteroclitic analogs of A2 epitopes, and rules for making such A2 heteroclitic analogs. However, no such rules or analogs have been developed for A3, A24, or B7 epitopes prior to the present invention.

Thus, in one aspect, the invention is directed to a method to enhance the immunogenicity of a peptide containing an epitope e.g. a B7 epitope, the method comprising i) providing a peptide comprising a first Class I epitope wherein said epitope comprises or consists essentially of an amino acid sequence having an amino-terminus and a carboxyl-terminus and at least one primary anchor residue, wherein amino acid residues of the epitope are numbered consecutively and the primary anchor residue nearest the amino-terminus of the epitope is at position 2 or position 3; and ii) introducing one or more conservative or semi-conservative substitution between the amino-terminus and the carboxyl-terminus of the epitope at position 3 and/or 5 and/or 7 which position does not contain a primary anchor residue, thereby constructing a peptide comprising a second Class I epitope which exhibits enhanced immunogenicity compared to the first Class I epitope.

In another aspect, in the case of B7 superfamily epitopes, the invention is directed to a method to enhance the immunogenicity of a peptide containing a B7 superfamily epitope, the method comprising i) providing a peptide comprising a first Class I epitope which is a B7 superfamily epitope wherein said epitope consists essentially of an amino acid sequence having an amino-terminus and a carboxyl-terminus and at least one primary anchor residue, wherein amino acid residues of the epitope are numbered consecutively and the primary anchor residue nearest the amino-terminus of the epitope is at position 2; and ii) introducing one or more conservative, semi-conservative, or non-conservative substitution between the amino-terminus and the carboxyl-terminus of the epitope at position 3 and/or 5 and/or 7, thereby constructing a peptide comprising a second Class I epitope which is a B7 superfamily epitope which exhibits enhanced immunogenicity compared to the first Class I epitope.

Thus, the invention relates to a method of producing a polypeptide comprising an analog of a MHC class I epitope, wherein the analog has enhanced immunogenicity compared to the epitope, comprising (a) identifying a MHC class I epitope comprising a formula (A), wherein formula (A) is Rn-R2-R3-R4-R5-R6-R7- . . . Rx, Rn is the N-terminal amino acid, Rx is the C-terminal amino acid, x=8-11 such that Rx can be from the eighth to the eleventh amino acid residue from Rn, R2 or R3 and Rx are primary anchor residues of a motif or supermotif, and (b) producing a polypeptide comprising an analog, said analog comprising a formula (B) identical to said formula (A) except one or more conservative or semiconservative amino acid substitutions at R3 and/or R5 and/or R7, provided said one or more substitutions is not of a primary anchor residues.

In some aspects, said analog comprises a formula (B) identical to said formula (A) except that R3 is Met, provided R3 is not an anchor residue of said motif or supermotif.

In some aspects, said analog comprises a formula (B) identical to said formula (A) except that R5 is Met.

In some aspects, said analog comprises a formula (B) identical to said formula (A) except that R7 is Met.

In some aspects, R3 is Ile in formula (A), and said analog comprises a formula (B) identical to said formula (A) except that R3 is Met.

In some aspects, R3 is Lys in formula (A), and said analog comprises a formula (B) identical to said formula (A) except that R3 is His or Leu.

In some aspects, R5 is Val in formula (A), and said analog comprises a formula (B) identical to said formula (A) except that R5 is His.

In some aspects, R5 is Leu in formula (A), and said analog comprises a formula (B) identical to said formula (A) except that R5 is Ile.

In some aspects, R5 is Val in formula (A), and said analog comprises a formula (B) identical to said formula (A) except that R5 is Ile or Phe.

In some aspects, R7 is His in formula (A), and said analog comprises a formula (B) identical to said formula (A) except that R7 is Trp.

In some aspects, R7 is Ala in formula (A), and said analog comprises a formula (B) identical to said formula (A) except that R7 is Pro.

In some aspects, R7 is Tyr in formula (A), and said analog comprises a formula (B) identical to said formula (A) except that R7 is His or Met.

In other aspects, the invention relates to a method of producing a polypeptide comprising an analog of a MHC class I epitope, wherein the analog has enhanced immunogenicity compared to the epitope, comprising (a) identifying a MHC class I epitope comprising a formula (A), wherein formula (A) is Rn-R2-R3-R4-R5-R6-R7- . . . Rx, Rn is the N-terminal amino acid, Rx is the C-terminal amino acid, x=8-11 such that Rx can be from the eighth to the eleventh amino acid residue from Rn, R2 or R3 and Rx are primary anchor residues of a motif or a supermotif, and (b) producing a polypeptide comprising an analog, said analog comprising a formula (B) identical to said formula (A) except one or more nonconservative amino acid substitutions at R3 and/or R5 and/or R7.

Thus, in some aspects, R7 is Tyr in formula (A), and said analog comprises a formula (B) identical to said formula (A) except that R7 is Gly, Glu, or Asp.

The second Class I epitope described above is generically referred to as a “heteroclitic analog” or an “analog.”

In a preferred embodiment, the heteroclitic analog exhibits at least about 50% increased potency for a specific T-cell compared to the corresponding wildtype Class I epitope. The analog may contain only one substitution, or may contain two or three, and the substitution may be conservative or semi-conservative or, in the case of a B7 superfamily epitope, non-conservative. The heteroclitic analog may induce both Th1 and Th2 cytokines when bound by an HLA Class I molecule and contacted with the relevant cytotoxic T-cell. Preferably, the Class I epitope comprises an HLA supermotif selected from the group consisting of A1, A2, A3, A24, B7, B27, B44, B58 and B62, more preferably, the Class I epitope comprises an A2 supermotif or a B7 supermotif, most preferably, an A2.1 motif (e.g. an A*0201), or a B7 motif (e.g. a B*0702 motif).

The class I epitope may be from a viral antigen, a tumor-associated antigen, a parasitic antigen, a bacterial antigen or a fungal antigen.

The supermotif may be A1, wherein R2 is a primary anchor residue and is either T, I, L, V, M or S, and Rx is either F, W, or Y.

The supermotif may be A2, wherein R2 is a primary anchor residue and is either L, I, V, M, A, T, or Q, and Rx is I, V, M, A, T, or L.

The supermotif may be A2.1, wherein R2 is a primary anchor and is either L, M, V, Q, I, A, or T, and Rx is either V, L, I, M, A, or T.

The supermotif may be A3, wherein R2 is a primary anchor residue and is either V, S, M, A, T, L, or I, and Rx is R or K.

The supermotif may be A24, wherein R2 is a primary anchor residue and is either Y, f, W, I, V, L, M, or T, and Rx is either F, I, Y, W, L, or M.

The supermotif may be B7, wherein R2 is a primary anchor residue and is P and Rx is either V, I, L, F, M, W, Y, or A.

In another aspect, the invention is directed to a method to enhance the immunogenicity of a peptide containing e.g. an A3 or A24 epitope, the method comprising: i) providing a peptide comprising a class I epitope, wherein said epitope comprises an amino acid sequence having an amino-terminus and a carboxyl-terminus and at least one primary anchor residue, wherein amino acid residues of the epitope are numbered consecutively and the primary anchor residue nearest the amino-terminus of the epitope is at position 2; and ii) introducing one or more conservative, semi-conservative, or non-conservative substitutions between the amino-terminus and the carboxyl-terminus of the epitope at position 3 and/or 4 and/or 5 and/or 6 and/or 7.

Thus, the invention relates to a method of producing a polypeptide comprising an analog of an MHC class I epitope, wherein the analog has enhanced immunogenicity compared to the epitope, comprising: (a) identifying an MHC class I epitope comprising a formula (A), wherein: formula (A) is Rn-R2-R3-R4-R5-R6-R7- . . . Rx, Rn is the N-terminal amino acid, Rx is the C-terminal amino acid, x=8-11 such that Rx can be from the eighth to the eleventh amino acid residue from Rn, R2 or R3 and Rx are primary anchor residues of a motif or supermotif; and (b) producing a polypeptide comprising an analog, said analog comprising a formula (B) identical to said formula (A) except for one or more conservative, semi-conservative, or non-conservative amino acid substitutions at R3 and/or R4 and/or R5 and/or R6 and/or R7.

In some embodiments, the heteroclitic analog exhibits at least about 20%, at least about 30%, or at least about 40% increased potency for a specific T cell compared to the corresponding wild-type class I epitope. In a preferred embodiment, the heteroclitic analog exhibits at least about 50% increased potency for a specific T cell compared to the corresponding wild-type class I epitope. In highly preferred embodiments, the heteroclitic analog exhibits at least about 50%, at least about 60%, at least about 70%, at least about 80%, at least about 90%, at least about 100% (i.e., at least about 2-fold), at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, or at least about 1000% (i.e., at least about 10-fold) increased potency for a specific T cell compared to the corresponding wild-type class I epitope.

The analog may contain only one substitution, or may contain two or three or four, and the substitution may be conservative, semi-conservative, or non-conservative. The heteroclitic analog may induce both Th1 and Th2 cytokines when bound by an HLA class I molecule and contacted with the relevant cytotoxic T cell. In one embodiment of the invention, the class I epitope comprises an HLA supermotif selected from the group consisting of A1, A2, A3, A24, B7, B27, B44, B58 and B62. In another embodiment, the class I epitope comprises an A2 or B7 supermotif, or an A3 or A24 supermotif. In yet another embodiment, the class I epitope comprises an A2 motif (e.g. an A*0201 motif), an A3 motif (e.g. an A*0301 motif), an A24 motif (e.g. an A*2402 motif), or a B7 motif (e.g. a B*0702 motif).

The class I epitope may be from a viral antigen, a tumor-associated antigen (e.g. CEA or MAGE-1, MAGE-2, MAGE-3, MAGE-11, and MAGE-A10), a parasitic antigen, a bacterial antigen or a fungal antigen. Preferably, the epitope is from CEA or MAGE2.

The invention also provides methods of inducing a human cytotoxic T cell response against a preselected class I peptide epitope, the method comprising providing the heteroclitic analog described above; and contacting a human CTL with the heteroclitic analog.

In some aspects, the step of contacting is carried out in vitro. In some aspects, the step of contacting is carried out by administering to a subject a nucleic acid molecule comprising a sequence encoding the heteroclitic analog polypeptide.

The invention also provides analog polypeptides produced by the method described above. Thus, the invention provides “analog polypeptides” which comprise or alternatively consist of an analog obtainable by the methods herein. In particular, and preferably, such analog polypeptides comprise an analog which consists of an amino acid sequence selected from the group consisting of SEQ ID NOs:2, 3, 5, 6, 8, 9, 11-19, 21-25, 44-48, 50, 51, 53, 54, 56, 58, 59, and 61-66. The analog polypeptide may contain 9-20 amino acids, preferably 9-16, more preferably 9-15, but may also contain only a total of 9, 10, 11, 12, 13 or 14 amino acids. The defined heteroclitic analog epitopes may be included in a longer polypeptide or protein which is a homopolymer of the same epitope (e.g., analog) or a heteropolymer which contains a variety of such epitopes (e.g., analogs) or the heteroclitic analog epitope in combination with wildtype epitopes. These peptides and proteins may be included in compositions which are designed for pharmaceutical use.

The analog polypeptides containing the heteroclitic analog epitopes may be combined with other components to enhance further or modulate their activity in eliciting an immune response. These additional components may be covalently bound or non-covalently included in a mixture.

Thus, the analog polypeptide may comprise a T helper peptide, a spacer or linker, a carrier, may be linked to a lipid, and/or may comprise one or more epitopes, or one or more additional analogs, or one or more additional amino acids.

Further, the heteroclitic analog polypeptide may be admixed or joined to a CTL epitope, or to an HTL epitope, especially where the HTL epitope is a pan-DR binding molecule. Thus, the invention includes an analog polypeptide comprising or consisting of an analog joined to a CTL epitope and/or an HTL epitope, such as a pan-DR epitope, and also includes a composition containing an analog polypeptide comprising and/or consisting of an analog and a CTL epitope and/or and HTL epitope such as a pan-DR epitope.

A composition containing the heteroclitic analog polypeptide may further comprise a liposome, wherein the analog polypeptide is on or within the liposome, or the analog polypeptide may be joined to a lipid. The composition may comprise an HLA heavy chain, β2-microglobulin, and streptavidin, which may form a complex, and the heteroclitic analog polypeptide may be bound to said complex, whereby a tetramer is formed. The composition may comprise an antigen presenting cell (APC), and the analog polypeptide may be on or within the APC, and/or the analog polypeptide may be bound to an HLA molecule on the APC. Thus, when a CTL that is restricted to the HLA molecule is present, a receptor of the CTL may bind to a complex of the HLA molecule and the analog polypeptide. The APC may be a dendritic cell. The composition may also comprise an HLA molecule, and the analog polypeptide may be bound by the HLA molecule. The composition may comprise a label—e.g., biotin, a fluorescent moiety, a non-mammalian sugar, a radiolabel, or a small molecule capable of binding a monoclonal antibody. The composition may comprise a suitable diluent and/or excipient.

The compositions described are useful for prophylactic, therapeutic, diagnostic, and prognostic purposes. For example, the compositions are useful in eliciting an immune response against the corresponding wild-type epitope. The active component heteroclitic analog polypeptides may be present in unit dosage form. Compositions useful in treating subjects may also comprise nucleic acid molecules that encode the analog polypeptides described above optionally including control sequences for their expression.

### BRIEF DESCRIPTION OF THE DRAWINGS

FIGS. 1A-1D. FIGS. 1A and 1B represent the results of testing a panel of analogs of CEA.691 and MAGE3.112 respectively for ability to induce IFN-γ production in the corresponding CTL. FIGS. 1C and 1D are the corresponding dose response curves for CEA.691 and MAGE3.112 heteroclitic analogs respectively.

FIGS. 2A-2C. FIG. 2A represents a primary screening of a panel of analogs of the CEA.61 epitope indicating that the P4, W4, I4, and K1 analogs showed higher stimulating activity than wild-type peptide. FIGS. 2B and 2C demonstrate the ability of the analogs to stimulate two CTL lines. When tested further with two CTL lines generated in donor x662, only the P4 analog stimulated equivalent IFN-γ production at a >10-fold lower peptide dose compared to wild-type peptide.

FIGS. 3A-3B. FIG. 3A represents a primary screening of a panel of analogs of the CEA.61 epitope indicating that the Y3, L7, M7, I7, D7, G7, C7, Y7, and N7 analogs showed higher stimulating activity than wild-type peptide. FIG. 3B demonstrates the ability of the analogs to stimulate CTLs. When tested further, the L7, M7, I7, D7, G7, and C7 analogs stimulated equivalent IFN-γ production at a >10-fold lower peptide dose compared to wild-type peptide.

FIGS. 4A-4B. FIG. 4A represents a primary screening of a panel of analogs of the MAGE2.156 epitope indicating that the T1, Y1, I3, E4, and L4 analogs showed higher stimulating activity than wild-type peptide. FIG. 4B demonstrates the ability of the analogs to stimulate CTLs. When these analogs were tested further, the I3 and E4 analogs stimulated equivalent IFN-γ production at a >10-fold lower peptide dose compared to wild-type peptide.

FIGS. 5A-5C. FIG. 5A represents a primary screening of a panel of analogs of the MAGE2.156 epitope indicated that the I3, L4, L6, and M6 analogs showed higher stimulating activity than wild-type peptide. FIGS. 5B and 5C demonstrates the ability of the analogs to stimulate CTLs. When these analogs were tested further on two CTL lines, the L4, L6, and M6 analogs stimulated equivalent IFNγ production at a >10-fold lower peptide dose compared to wild-type peptide.

FIG. 6. FIG. 6 represents a primary screening of a panel of analogs of the MAGE 2.157 epitope indicating that the I5 and F5 analogs showed higher stimulating activity than wild-type peptide.

FIGS. 7A-7B. FIGS. 7A and 7B show dose response curves of heteroclitic analogs of MAGE2.157 in comparison to wild-type with regard to their ability to induce IFNγ production or IL-10 production from the appropriate CTLs.

FIGS. 8A-8C. FIGS. 8A-8C show the results of stimulation of CTL activity against endogenous peptide using various heteroclitic analogs of MAGE3.112.

FIGS. 9A-9B. FIGS. 9A-9B show the results of testing a panel of potential heteroclitic analogs of the epitope MAGE2.170 with respect to IFN-γ production from appropriate CTLs.

FIG. 10. FIG. 10 shows dose response curves of heteroclitic analogs of MAGE2.170 in comparison to wild-type with regard to their ability to induce IFN-γ.

FIG. 11. FIG. 11 shows the results of testing panels of analogs of HBVPol.455 epitope analogs with respect to the ability of these analogs to induce IFN-γ production in the corresponding CTLs.

FIGS. 12A-12B. FIG. 12A shows the results of testing panels of analogs of HIVPol.476 epitope analogs with respect to the ability of these analogs to induce IFN-γ production in the corresponding CTLs. FIG. 12B shows the relevant dose response curve for the successful HIVPol.476 analogs.

FIGS. 13A-13B. FIGS. 13A and 13B show the dose response curves for wildtype and a heteroclitic analog of HBVPol.455 to produce IFN-γ and IL-10 in appropriate CTLs.

FIG. 14. FIG. 14 shows the results of testing a panel of potential heteroclitic analogs of the epitope p53.149M2 with respect to IFN-γ production from appropriate CTLs.

FIGS. 15A-15B. FIGS. 15A and 15B are-the corresponding dose response curves for production of IFN-γ and IL-10 by successful heteroclitic analogs of p53.149M2.

FIG. 16. FIG. 16 shows the results of testing a panel of potential analogs of the p53.Mu184 epitope for IFN-γ production in CTLs.

FIG. 17. FIG. 17 shows the dose response curves for wildtype and two successful heteroclitic analogs of p53.Mu184 with respect to IFN-γ production.

FIGS. 18A-18D. FIGS. 18A-18D show the cross-reactivity of heteroclitic analogs with regard to the corresponding wildtype epitope. In FIGS. 18A and 18B, IFN-γ production is plotted as a function of concentration using stimulation by the immunizing peptide. FIGS. 18C and 18D show the corresponding results when wildtype epitope is used as the stimulant as opposed to the heteroclitic analog used for the initial induction of CTL.

FIG. 19. FIG. 19 shows the IFN-γ release with respect to stimulation by p53.261 and its heteroclitic analogs.

FIG. 20. FIG. 20 shows ELISPOT results with respect to various p53.261 heteroclitic analogs.

### DETAILED DESCRIPTION OF THE INVENTION

1. Overview—Analogs

The present invention relates to methods of designing heteroclitic analogs that bind to HLA class I molecules, and methods of producing polypeptides comprising or consisting of such analogs, and also relates to the polypeptides themselves, as well as polynucleotides encoding such polypeptides. Immunization with heteroclitic analogs is a more effective and efficient strategy for vaccination against tumors especially where raising effective CTLs has so far proven to be a challenge.

“Heteroclitic analogs,” as described herein, are epitopes with 1, 2, 3, or 4 amino acid substitutions that result in increased potency for a specific T cell, as measured by increased responses to a given dose, or by a requirement of lesser amounts to achieve the same response as a homologous class I peptide. The methods of the invention are useful to modify any class I peptide, particularly those associated with human cancers and precancerous conditions, and from infectious agents such as viruses, bacteria, fungi, and protozoan parasites.

Importantly, the phenomenon of heteroclicity applies across HLA molecules that bind a particular class I peptide. For example, a heteroclitic analog peptide bearing the A2 supermotif is heteroclitic across all HLA molecules in the HLA-supertype (e.g. A*0201, A*0202, A*0203, A*0204, A*0205, A*0206, A*0207, etc.; see Table 5). Similarly, a heteroclitic analog peptide bearing the A3 supermotif is heteroclitic across all HLA molecules in the HLA-supertype (e.g. A*0301, A*1101, A*3101, A*6801, etc., see Table 5). A heteroclitic analog peptide bearing the A24 supermotif is heteroclitic across all HLA molecules in the HLA-supertype (e.g. A*2301, A*2402, A*3001, etc., see Table 5). Also, a heteroclitic analog peptide bearing the B7 supermotif is heteroclitic across all HLA molecules in the HLA-supertype (e.g., B*0702, B*0703, B*0704, B*0705, B*1508, B*3501, B*3502, B*3503, B*3503, B*3504, B*3505, B*3506, B*3507, B*3508, B*5101, B*5102, B*5103, B*5104, B*5105, B*5301, B*5401, B*5501, B*5502, B*5601, B*5602, B*6701, B*7801, etc.; see Table 5). Thus, a heteroclitic analog peptide bearing a different sequence motif (e.g., A1, A2, A3, A24, B7, B27, B44, B58, B62, etc.) induces a more potent immune response across all HLA molecules within their specific HLA superfamily.

The inventors have found specific rules governing the design of heteroclitic analogs for certain HLA supertypes which enhance the immune response to the corresponding wild-type epitope. These rules are applicable with respect to epitopes bearing motifs or supermotifs which bind to HLA molecules encoded by any class I allele. By using these rules, it is possible to enhance the immunogenicity, therefore, of any “wild-type” or “native” class I epitope.

Briefly, for A2 superfamily epitopes, the rules state that the wild-type class I epitope is modified by substituting a conservative or semi-conservative amino acid at position 3 and/or 5 and/or 7 of the epitope. For B7 superfamily epitopes, the rule states that the wild-type class I epitope is modified by substituting a conservative or semi-conservative or non-conservative amino acid at position 3 and/or 5 and/or 7 of the epitope. The nature of the conservative, semi-conservative, or non-conservative amino acid to be substituted is defined by the description in Preparation B hereinbelow, the results of which are summarized in Table 2. Thus, by consulting Table 2, one can determine suitable candidates for substitution at these positions. As shown in Table 2, each of the amino acids shown across the top of the table bears a numerically defined relationship to the remaining 19 genetically encoded amino acids. The lower the index, the higher the conservation; the same amino acid will have a similarity assignment of 1.0; maximally different amino acids will have similarity assignments approaching 20. Using the method set forth in Preparation B, amino acids which are not gene-encoded can also be assigned similarity indices and can be classified with respect to any natively occurring amino acid as conservative or semi-conservative (or non-conservative).

The inventors have identified novel A3 and A24 superfamily analogs, i.e., heteroclitic analogs, that do not follow the previously established rules. Such analogs are presented in Table 6, and in SEQ ID NOs:11-19 and 21-25. Surprisingly, these analogs contain, in addition to a conservative or semi-conservative substitution at position 3 and/or 7 of the epitope, a conservative or non-conservative substitution at position 4 and/or 6 of the epitope.

In particular, the inventors identified analogs of the A3 superfamily epitope CEA.61 (SEQ ID NO:10) (shown in Table 6). Unlike A2 and B7 heteroclitic epitopes, heteroclitic analogs of the A3 superfamily epitope could be generated by introducing substitutions both at odd-number positions in the middle of the peptide (position 7), and at even-numbered positions (position 4). The substitutions at position 4 for the CEA.61 epitope were conservative or non-conservative (G→P or I) (SEQ ID NOs:11-12), while the substitutions at position 7 were conservative, semi-conservative, or non-conservative (W→L, M, I, D, G, C, or N) (SEQ ID NOs:13-19).

The inventors also identified analogs of the A24 superfamily epitope MAGE2.156 (SEQ ID NO:20) (shown in Table 6). Similar to the A3 superfamily epitopes, heteroclitic analogs of the A24 superfamily epitope could be generated by introducing substitutions at both even and odd-numbered positions in the middle of the peptide. The substitutions were conservative (position 3 L→I, position 4 Q→E, position 6 V→M or L) or non-conservative (position 4 Q→L) (SEQ ID NOs:21-25). Thus, the observation that substitutions at even-numbered positions can result in heteroclitic analogs for the MAGE2.156 epitope indicate a partially overlapping substitution pattern with that observed for A3 superfamily epitopes.

Heteroclitic analog peptides of the invention are particularly useful to induce an immune response against antigens to which a subject's immune system has become tolerant. Human subjects are particularly preferred, but the methods can also be applied to other mammals such as transgenic laboratory mice that express HLA, taking into account the corresponding HLA motifs with regard to these subjects.

Tolerance refers to a specific immunologic nonresponsiveness induced by prior exposure to an antigen. Tolerance can be overcome by identifying a particular class I peptide epitope to which a patient is tolerant, modifying the peptide epitope sequence according to the methods of the invention, and inducing an immune response that cross-reacts against the tolerized epitope (antigen). Overcoming tolerance is particularly desirable, for example, when the immune system of the subject is tolerant of a viral or tumor-associated antigen, the latter antigens often being over-expressed self-proteins as a consequence of cell transformation.

To determine rules for designing heteroclitics, several different CTL lines were screened for reactivity against panels of analogs. Modification of T cell stimulatory capacity was achieved with no alternation of the primary MHC anchors.

The wildtype epitopes include tumor epitopes derived from self antigens that are specifically up-regulated in epithelial cell cancers and have been shown to be immunogenic. Viral epitopes used, such as those from the polymerase genes of the HIV and HBV, have been shown to be immunogenic as well.

The rules described herein provide a basis to design heteroclitic analogs, drastically reducing the screening otherwise required and are extremely useful in designing epitope-based vaccines for cancer and infectious diseases.

In the examples set forth below, 17% of the total analogs screened (which fit the heteroclicity rules disclosed herein) were heteroclitic (16/95). This is significant for two reasons: first, the efficiency of detecting heteroclitics increased from 2.2% to 17% by employing analogs that follow the rules of heteroclitic substitution; second, the number of peptides which need to be synthesized is reduced dramatically from about a 100 analogs per epitope to about 15 analogs per epitope, making the process cost effective and amenable to high throughput. Through the application of the heteroclitic substitution rules of the invention, the efficiency of generating heteroclitic analogs was increased nearly 100 to 1000-fold, from 0.2% (4 identified from screening of 233 CEA.691 and MAGE3.112 analogs) to 33% (3 identified by screening of 9 predicted analogs). The latter frequency may be a gross underestimate since only 4 of 6 analogs showing potential heteroclitic activity in initial assays were subjected to further analysis.

Previous studies showed that modulation of T cell responses by heteroclitic analogs involved TCR contact residues (Byrne, et al., J. Immunol. 51:682 (1984), McMichael, et al., N. England. J. Med. 309:13 (1983), Zugel, et al., J. Immunol. 161:1705 (1998), Rivoltini, et al., Cancer Research 59:301 (1999)), but the present study did not find this. Binding analyses performed on the analogs indicated that there is an alteration in MHC binding for the better or worse in a majority of cases (80%). Out of the 13 analogs which were tested for HLA-A2 binding, ten analogs had alteration in MHC binding, with six analogs binding better than wild-type peptides and four analogs that bound worse than wild-type, but still generated a substantially increased biological response.

Some studies modify primary MHC anchor residues in order to increase MHC binding (this approach has been used by some groups to generate analogs (Pfeiffer, et al., J. Exp. Med. 181:1569 (1995), Valmori, et al., J. Immunol. 160:1150-1758 (1998), Parkhurst, et al., J. Immunol. 157:2539 (1996)). Increased biological responses without changing primary TCR contact residues or primary MHC anchor residues was observed in this study. Since increased responses were mediated with alteration in MHC binding, it is postulated that the effect may be mediated by changing secondary anchor positions. More evidence supporting this comes from the finding that heteroclitic substitutions for HLA-A2.1 and -B7 occur at odd numbered positions (3, 5, 7) in the middle of the peptide. All these positions, particularly positions 3, 5, and 7, have been shown to be secondary anchor positions for binding to the HLA-A2 molecule (Ruppert, et al., Cell 74:929 (1993), Madden, Annu. Rev. Immunol. 13:587-622 (1995)). Two of these positions (3 and 7) have been shown to be secondary anchor positions for binding to HLA-A2.1 molecule by several groups (Ruppert, et al., Cell 74:929 (1993), Madden, Annu. Rev. Immunol. 13:587-622 (1995)).

Alteration of such secondary anchor positions can translate into T cell recognition differences (Valmori, et al., J. Immonol. 160:1750 (1998); Davis, et al., Annu. Rev. Immunol. 16:523 (1998)), however in these studies T cell recognition differences were associated with changes in MHC binding and no rules were defined for the kinds of amino acid substitutions involved in obtaining heteroclicity. The mechanism by which such a translation from changing secondary anchors to a change in T cell recognition takes place is currently unclear. However, some models suggest that changes in the way residues at secondary anchor positions engage the MHC may lead to alteration in the orientation or increased flexibility of TCR contact residues, resulting in enhancement of the binding of these analogs to the TCR (Kersh, et al., J. Exp. Med. 184:1259 (1996), Evavold, et al., J. Immunol. 148:347 (1992), Alam, et al., Immunity 10:227 (1999), Hampl, et al., Immunity 7:379-85 (1997)). Also, some previous studies implied that modulation of T cell responses by heteroclitic analogs directly involve main TCR contact residues (Zaremba, et al., Cancer Research 57:4570 (1997), Loftus, et al., Cancer Research 58:2433 (1998), Dressel, et al., J. Immunol. 159:4943 (1997)). This finding, however, is not corroborated by the current systematic analysis. The enhanced T cell recognition against analogs identified in the present study is not likely due to increases in MHC binding capacity, though increased binding is likely to play an important role in the case of analogs in which primary anchor positions have been optimized. The present study suggests that heteroclitic analogs are most likely generated by subtle alterations in conformation rather than by gross alterations of TCR or MHC binding capacity.

Differential regulation of production of Th1 or Th2 cytokines was not observed. Instead, the present data suggested that the heteroclitic analogs increased the production of both Th1 and Th2 responses, although the magnitude and kinetics of the increase may be different. In fact, some groups (Nicholson, et al., Int. Immunol. 12(2):205-13 (2000), Parkhurst, et al., J. Immunol. 157:2539 (1996)) have recently reported such overall stimulation by peptide analogs. This is attributable to a stronger TCR signal induced by analogs, though the mechanism of such overall stimulation remains to be elucidated.

The efficacy of heteroclitic analogs in vivo using relevant tumor models or models in which tolerance to self antigens exists is evaluated. Accordingly, it is found that immunization with heteroclitic analogs is a more effective and efficient strategy for vaccination against tumors where raising effective CTLs has so far proved to be a challenge.

In a set of experiments, Applicants have identified heteroclitic analogs of a number of different HLA-A2.1-restricted CTL epitopes of cancer and viral origin. The relevant wildtype epitopes are shown in Table 1. All these epitopes have been shown to be immunogenic in our earlier reports (Kawashima, et al., Human Immunology 59:1-14 (1998), Ishioka, et al., J. Immunol. 162(7):3915-25 (1999)). In initial experiments, the antigenicity of 233 analogs of the CEA.691 and MAGE3.112 CTL epitopes was investigated. The nature of the four heteroclitic analogs identified suggested that heteroclitic substitutions involved conservative substitutions at positions 3, 5 and 7. This hypothesis, was tested in a subsequent study involving three additional epitopes MAGE2.157, HIVPol.476, and HBVPol.455. All of the heteroclitic analogs thus identified conformed to the rules proposed, namely that heteroclitic analogs were associated with conservative or semi-conservative substitutions at positions 3, 5 and/or 7.

To more closely mimic the clinical application of heteroclitic analogs in cancer immunotherapy, the murine epitope, p53.261 was also modified. A partial state of T cell tolerance has been reported for this epitope (Theobald, et al., Proc. Natl. Acad. Sci. 92:11993-11997 (1995), Theobald, et al., J. Exp. Med., 185(5):833-841 (1997)). Four out of nine predicted p53.261 analogs were found to induce stronger analog-specific CTL responses in vivo compared to the CTL responses induced by the native peptide. More significantly, when the cross-reactivity of the CTL raised by immunization with heteroclitic analogs was analyzed, three p53.261 analogs induced CTL which responded vigorously against the native p53.26epitope. Finally, the relevance of these findings for human CTL was addressed by demonstrating that heteroclitic analogs of the MAGE3.112 epitope are immunogenic for human T cells in vitro. The resulting CTL can recognize wildtype naturally processed antigen in the form of tumor cell lines.

The studies presented herein demonstrate that heteroclicity is a global phenomenon, as heteroclitic analogs were identified for all the epitopes studied. In addition, the present application shows that it is possible to detect heteroclitic analogs both in clonal T cell populations (as has been described earlier studies) as well as in bulk T cell populations following in vivo immunization. Moreover, it is demonstrated herein that heteroclicity (both in the HLA A2.1 system as well as for other Class I supermotifs) is associated with discrete structural features which allow rational prediction of heteroclicity.

It is demonstrated, further that p53.261 heteroclitic analogs induce CTLs with higher avidity and also induced these cells in greater numbers (precursor frequency) than those induced with wildtype peptide; heteroclitic CTL induction in vivo, and its application to breaking T cell tolerance is demonstrated.

The heteroclitic analogs were effective in raising bulk populations of specific T cells following in vivo immunization. Polyclonal responses that bear TCR from multiple TCR genes, are more efficacious in resolving disease states in a clinical setting. Finally, the ability to generate high precursor frequencies of CTL possessing strong cross-reactive avidity against wildtype epitope is important in instances where effective CTL responses against epitopes, normally tolerant to the immune system, are required.

In another set of experiments, applicants identified heteroclitic analogs of the B7 superfamily epitope MAGE2.170 (shown in Table 1). Like A2 heteroclitic epitopes, heteroclitic analogs of the B7 superfamily epitope could be generated by introducing substitutions at an odd-number position in the middle of the peptide (position 7). The nature of the substitutions for the MAGE2.170 epitope were either conservative/semi-conservative (the Y→H and Y→M substitutions) or non-conservative (the Y→E, Y→G, and Y→D substitutions) compared to the native residue (Table 8). Thus, the observation that non-conservative substitutions can result in heteroclitic analogs for the MAGE2.170 CTL epitope indicate a partially overlapping substitution pattern than that observed with A2 superfamily epitopes.

2. Definitions

A “Human Leukocyte Antigen” or “HLA” is a human class I or class II Major Histocompatibility Complex (MHC) protein (see, e.g., Stites, et al., IMMUNOLOGY, 8^{TH} ED., Lange Publishing, Los Altos, Calif. (1994).

With regard to a particular amino acid sequence, an “epitope” is a set of amino acid residues which is involved in recognition by a particular immunoglobulin, or in the context of T cells, those residues necessary for recognition by T cell receptor proteins when presented in the context of an HLA. In an immune system setting, in vitro or in vivo, an epitope is the collective features of a molecule, such as primary, secondary and tertiary peptide structure, and charge, that together form a site recognized by an immunoglobulin, T cell receptor or HLA molecule. “Epitope” refers to both CTL and HTL epitopes.

A “class I epitope” or “CTL epitope” refers to a peptide that binds to a class I HLA molecule. As described herein, a class I epitope is typically about 8 to about 13 amino acids in length, and may be 8, 9, 10, 11, 12, or 13 amino acids in length. Binding to the HLA molecule is primarily controlled by two primary anchor residues, one of which is at the carboxyl-terminus of the epitope and the other of which is at positions 2 or 3. Binding may also be aided by one or more secondary anchor residues. For the convenience of the reader, various primary HLA class I binding anchors are set forth in Table 3. The pattern of anchors is referred to as a “motif.” A “supermotif” is a peptide with binding specificity shared by HLA molecules encoded by two or more HLA alleles. Preferably, a supermotif-bearing peptide is recognized with high or intermediate affinity (as defined herein) by two or more HLA antigens. Examples of class I supermotifs include, e.g., A1, A2, A3, A24, B7, B27, B44, B58 and B62 (See “Class I Motifs” section and Tables 3-5).

The supermotif may be A1, wherein R2 is a primary anchor residue and is T, I, L, V, M or S, and Rx is F, W, or Y.

The supermotif may be A2, wherein R2 is a primary anchor residue and is L, I, V, M, A, T, or Q, and Rx is I, V, M, A, T, or L.

The supermotif may be A2.1, wherein R2 is a primary anchor and is L, M, V, Q, I, A, or T, and Rx is V, L, I, M, A, or T.

The supermotif may be A3, wherein R2 is a primary anchor residue and is V, S, M, A, T, L, or I, and Rx is R or K.

The supermotif may be A24, wherein R2 is a primary anchor residue and is Y, F, W, I, V, L, M, or T, and Rx is F, I, Y, W, L, or M.

The supermotif may be B7, wherein R2 is a primary anchor residue and is P and Rx is V, I, L, F, M, W, Y, or A.

In one embodiment, the supermotif is A2 or B7.

In another embodiment, the supermotif is A3 or A24.

The class I epitope may be from a viral antigen, a tumor-associated antigen (e.g. CEA or MAGE-1, MAGE-2, MAGE-3, MAGE-11, and MAGE-A10), a parasitic antigen, a bacterial antigen or a fungal antigen.

Examples of suitable tumor-associated antigens include prostate specific antigens (PSA), melanoma antigens MAGE-1, MAGE-2, MAGE-3, MAGE-11, MAGE-A10, as well as BAGE, GAGE, RAGE, MAGE-C1, LAGE-1, CAG-3, DAM, MUC1, MUC2, MUC18, NY-ESO-1, MUM-1, CDK4, BRCA2, NY-LU-1, NY-LU-7, NY-LU-12, CASP8, RAS, KIAA-2-5, SCCs, p53, p73, CEA, Her 2/neu, Melan-A, gp100, tyrosinase, TRP2, gp75/TRP1, kallikrein, prostate-specific membrane antigen (PSM), prostatic acid phosphatase (PAP), prostate-specific antigen (PSA), PT1-1, β-catenin, PRAME, Telomerase, FAK, cyclin D1 protein, NOEY2, EGF-R, SART-1, CAPB, HPVE7, p15, Folate receptor CDC27, PAGE-1, and PAGE-4. Examples of suitable infectious disease-associated antigens include hepatitis B core and surface antigens (HBVc, HBVs), hepatitis C antigens, Epstein-Barr virus antigens, human immunodeficiency virus (HIV) antigens and human papilloma virus (HPV) antigens, Mycobacterium tuberculosis and Chlamydia. Examples of suitable fungal antigens include those derived from Candida albicans, Cryptococcus neoformans, Coccidoides spp., Histoplasma spp, and Aspergillus fumigatis. Examples of suitable protozoan parasitic antigens include those derived from Plasmodium spp., including P. falciparum, Trypanosoma spp., Schistosoma spp., Leishmania spp and the like.

Throughout this disclosure, “binding data” results are often expressed in terms of “IC₅₀'s.” IC₅₀ is the concentration of peptide in a binding assay at which 50% inhibition of binding of a reference peptide is observed. Given the conditions in which the assays are run (i.e., limiting HLA proteins and labeled peptide concentrations), these values approximate Kd values. Assays for determining binding are described in detail, e.g., in PCT publications WO 94/20127 and WO 94/03205, incorporated herein by reference. It should be noted that IC₅₀ values can change, often dramatically, if the assay conditions are varied, and depending on the particular reagents used (e.g., HLA preparation, etc.). For example, excessive concentrations of HLA molecules will increase the apparent measured IC₅₀ of a given ligand. Alternatively, binding is expressed relative to a reference peptide. Although as a particular assay becomes more, or less, sensitive, the IC₅₀'s of the peptides tested may change somewhat, the binding relative to the reference peptide will not significantly change. For example, in an assay run under conditions such that the IC₅₀ of the reference peptide increases 10-fold, the IC₅₀ values of the test peptides will also shift approximately 10-fold. Therefore, to avoid ambiguities, the assessment of whether a peptide is a good, intermediate, weak, or negative binder is generally based on its IC₅₀, relative to the IC₅₀ of a standard peptide. Binding may also be determined using other assay systems known in the art.

The designation of a residue position in an epitope as the “carboxyl or C-terminus” refers to the residue position at the end of the epitope which is nearest to the carboxyl-terminus of a peptide, which is designated using conventional nomenclature as defined below. The “C-terminus” of the epitope may or may not actually correspond to the end of the peptide or polypeptide.

The designation of a residue position in an epitope as “N-terminus” or “amino-terminal position” refers to the residue position at the end of the epitope which is nearest to the N-terminus of a peptide, which is designated using conventional nomenclature as defined below. The “N-terminus” of the epitope may or may not actually correspond to the end of the peptide or polypeptide.

A “computer” or “computer system” generally includes: a processor; at least one information storage/retrieval apparatus such as, for example, a hard drive, a disk drive or a tape drive; at least one input apparatus such as, for example, a keyboard, a mouse, a touch screen, or a microphone; and display structure. Additionally, the computer may include a communication channel in communication with a network. Such a computer may include more or less than what is listed above.

“Heteroclitic analogs,” as described herein, are epitopes with 1, 2, 3, or 4 amino acid substitutions that result in increased potency for a specific T cell, as measured by increased responses to a given dose, or by a requirement of lesser amounts to achieve the same response as a homologous (“wild type”) class I epitope. The methods of the invention are useful to modify any class I epitope, particularly those associated with human cancers and precancerous conditions, and from infectious agents such as viruses, bacteria, fungi, and protozoan parasites. Heteroclitic analogs are also referred to herein as “analogs.”

In a preferred embodiment, the heteroclitic analog exhibits at least about 50% increased potency for a specific T cell compared to the corresponding wild-type class I epitope. The analog may contain only one substitution, or may contain two or three or four, and the substitution may be conservative, semi-conservative, or non-conservative. The heteroclitic analog may induce both Th1 and Th2 cytokines when bound by an HLA class I molecule and contacted with the relevant cytotoxic T cell. Preferably, the class I epitope comprises an HLA supermotif selected from the group consisting of A1, A2, A3, A24, B7, B27, B44, B58 and B62, more preferably, the class I epitope comprises an A2 or B7 supermotif, or an A3 or A24 supermotif, most preferably, an A2 motif (e.g. an A*0201 motif), an A3 motif (e.g. an A*0301 motif), an A24 motif (e.g. an A*2402 motif), or a B7 motif (e.g. B*0702).

Thus, the invention relates to a method of producing a polypeptide comprising an analog of a MHC class I epitope, wherein the analog has enhanced immunogenicity compared to the epitope, comprising: (a) identifying a MHC class I epitope comprising a formula (A), wherein: formula (A) is Rn-R2-R3-R4-R5-R6-R7- . . . Rx, Rn is the N-terminal amino acid, Rx is the C-terminal amino acid, x=8-11 such that Rx can be from the eighth to the eleventh amino acid residue from Rn, R2 or R3 and Rx are primary anchor residues of a motif or supermotif; and (b) producing a polypeptide comprising an analog, said analog comprising a formula (B) identical to said formula (A) except for one or more conservative, semi-conservative, or non-conservative amino acid substitutions at R3 and/or R4 and/or R5 and/or R6 and/or R7 and/or R8 and/or R9 and/or R10, provided that, in some embodiments, said one or more substitutions is not of a primary anchor residue.

As used herein amino acids that are “conserved” or “conservative,” and “semi-conserved” or “semi-conservative,” and “non-conserved” or “non-conservative” are defined in accordance with Preparation B and set forth in Table 2.

As used herein, “high affinity” with respect to HLA class I molecules is defined as binding with an IC₅₀, or K_{D} value, of 50 nM or less; “intermediate affinity” is binding with an IC₅₀ or K_{D} value of between about 50 and about 500 nM. “High affinity” with respect to binding to HLA class II molecules is defined as binding with an IC₅₀ or K_{D} value of 100 nM or less; “intermediate affinity” is binding with an IC₅₀ or K_{D} value of between about 100 and about 1000 nM.

The invention also provides analog polypeptides produced by the method described above. Thus, the invention provides “analog polypeptides” which comprise or alternatively consist of an analog obtainable by the methods herein. Such analog polypeptides may also be referred to herein as analog “proteins” and “peptides” and other equivalent phrases. In particular, and preferably, such analog polypeptides comprise an analog which consists of an amino acid sequence selected from the group consisting of SEQ ID NOs: 2, 3, 5, 6, 8, 9, 11-19, 21-25, 44-48, 50, 51, 53, 54, 56, 58, 59, and 61-66. These analog peptides and proteins may be included in compositions which, in some embodiments, are designed for pharmaceutical use.

By “analog polypeptides” is meant all forms of analog proteins and polypeptides described herein. The analog polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The invention also provides analog polynucleotides, which encode the analog polypeptides of the invention. Thus, the invention provides “analog polynucleotides” which comprise or alternatively consist of a nucleic acid encoding an analog obtainable by the methods herein. Such analog polynucleotides may also be referred to herein as analog “nucleic acid molecules” and other equivalent phrases. In particular, and preferably, such analog polynucleotides comprise a nucleic acid encoding an analog which consists of an amino acid sequence selected from the group consisting of SEQ ID NOs: 2, 3, 5, 6, 8, 9, 11-19, 21-25, 44-48, 50, 51, 53, 54, 56, 58, 59, and 61-66. These analog polynucleotides may be included in compositions which, in some embodiments, are designed for pharmaceutical use.

By “analog polynucleotides” is meant all forms of analog polynucleotides described herein. The analog polynucleotides can be prepared in any suitable manner. Such polynucleotides include isolated naturally occurring polynucleotides, recombinantly produced polynucleotides, synthetically produced polynucleotides, or polynucleotides produced by a combination of these methods. Means for preparing such polynucleotides are well understood in the art.

The phrases “isolated” or “biologically pure” refer to material that is substantially or essentially free from components which normally accompany the material as it is found in its native state. Thus, isolated polypeptides and polynucleotides in accordance with the invention preferably do not contain materials normally associated with the polypeptides or polynucleotides in their in situ environment.

An “HTL epitope” or “T helper epitope” or “class II peptide” refers to a peptide that binds to a class II HLA molecule. An HTL epitope is a peptide that comprises an allele-specific class II motif typically about 6 to about 25 amino acids in length. Such peptides will bind an HLA molecule and induce an HTL response. Thus, an HTL epitope is capable of binding to an appropriate HLA class II molecule and thereafter inducing a helper T cell response.

A “Pan-DR binding peptide” (e.g., PADRE® peptide, Epimmune Inc., San Diego, Calif.) is a type of HTL epitope and is a member of a family of molecules that binds more that one HLA class II DR molecule. The pattern that defines the PADRE® family of molecules can be thought of as an HLA class II supermotif. Peptides comprising the pattern found in PADRE® molecules bind to most HLA-DR molecules and stimulate in vitro and in vivo human helper T lymphocyte (HTL) responses.

A “composition” contains one or more analog polypeptides and/or analog polynucleotides of the invention and another component such as an excipient, a diluent, a non-analog polypeptide (e.g., a polypeptide comprising a CTL epitope, an HTL epitope such as a pan-DR binding peptide, and/or a carrier, etc.), a polynucleotide encoding such a non-analog polypeptide, a lipid, or a liposome, as well as other components described herein. There are numerous embodiments of compositions in accordance with the invention, such as a cocktail of one or more analog polypeptides and/or analog polynucleotides; one or more analogs and one or more CTL and/or HTL epitopes; and/or nucleic acids that encode such peptides or polypeptides, e.g., a minigene that encodes a polyepitopic analog polypeptide.

The polypeptides or polynucleotides of the invention can optionally be modified, such as by lipidation, addition of targeting or other sequences. Polypeptides of the invention can be admixed with, or linked to, HLA class II-binding peptides, to facilitate activation of both cytotoxic T lymphocytes and helper T lymphocytes.

“Pharmaceutically acceptable” refers to a generally non-toxic, inert, and/or physiologically compatible composition.

As used herein, a “vaccine” is a pharmaceutically acceptable composition that contains one or more analog polypeptides and/or analog polynucleotides of the invention. Compositions, especially vaccines, can also comprise peptide-pulsed antigen presenting cells, e.g., dendritic cells.

The invention is set forth in further detail below.

3. A2, A3, A24, and B7 Analogs and Methods of Preparation

The invention provides methods to prepare peptides containing epitopes which have enhanced ability to effect an immune response with respect to corresponding analogous wild-type epitopes. The resulting “heteroclitic analogs” are useful in immunological compositions for treatment of viral diseases, cancer, and other conditions which are characterized by displayed antigens on target cells.

Thus, in one aspect, the invention is directed to a method to enhance the immunogenicity of a peptide containing an epitope, the method comprising: i) providing a peptide comprising a class I epitope, wherein said epitope comprises an amino acid sequence having an amino-terminus and a carboxyl-terminus and at least one primary anchor residue, wherein amino acid residues of the epitope are numbered consecutively and the primary anchor residue nearest the amino-terminus of the epitope is at position 2 or position 3; and ii) introducing one or more conservative, semi-conservative, or non-conservative substitutions between the amino-terminus and the carboxyl-terminus of the epitope at position 3 and/or 4 and/or 5 and/or 6 and/or 7 which position does not contain a primary anchor residue.

In a preferred embodiment, the heteroclitic analog exhibits at least about 50% increased potency for a specific T cell compared to the corresponding wild-type class I epitope. In a more preferred embodiment, the heteroclitic analog stimulates an equivalent CTL response (i.e. IFNγ release) as the wild-type peptide at a greater than or equal to 10-fold lower dose.

The analog may contain only one substitution, or may contain two or three or four, and the substitution may be conservative, semi-conservative, or non-conservative. The heteroclitic analog may induce both Th1 and Th2 cytokines when bound by an HLA class I molecule and contacted with the relevant cytotoxic T cell. Preferably, the class I epitope comprises an HLA supermotif selected from the group consisting of A1, A2, A3, A24, B7, B27, B44, B58 and B62, more preferably, the class I epitope comprises comprises an A2 or B7 supermotif, or an A3 or A24 supermotif, most preferably, an A2 motif (e.g. an A*0201 motif), an A3 motif (e.g. an A*0301 motif), an A24 motif (e.g. an A*2402 motif), or a B7 motif (e.g. B*0702) (See “Class I Motifs” section below, and Tables 3-5).

Class I epitopes that serve as the corresponding “wild-type” epitope can be derived from any proteinaceous source. For example, the class I peptides can be derived from viral antigens, tumor-associated antigens, parasitic antigens, bacterial antigens or fungal antigens. The class I epitope may be from a viral antigen (e.g. HBV or HIV), a tumor-associated antigen (e.g. CEA or MAGE-1, MAGE-2, MAGE-3, MAGE-11, MAGE-A10, or p53), a parasitic antigen, a bacterial antigen or a fungal antigen. The wild-type epitopes include tumor epitopes derived from self-antigens that are specifically up-regulated in epithelial cell cancers and have been shown to be immunogenic. Viral epitopes used, such as those from the polymerase genes of the HIV and HBV, have been shown to be immunogenic as well.

Thus, heteroclitic analogs based on epitopes from a number of potential antigens can be used in the present invention. Examples of suitable tumor-associated antigens include prostate specific antigens (PSA), melanoma antigens MAGE-1, MAGE-2, MAGE-3, MAGE-11, MAGE-A10, as well as BAGE, GAGE, RAGE, MAGE-C1, LAGE-1, CAG-3, DAM, MUC1, MUC2, MUC18, NY-ESO-1, MUM-1, CDK4, BRCA2, NY-LU-1, NY-LU-7, NY-LU-12, CASP8, RAS, KIAA-2-5, SCCs, p53, p73, CEA, Her 2/neu, Melan-A, gp100, tyrosinase, TRP2, gp75/TRP1, kallikrein, prostate-specific membrane antigen (PSM), prostatic acid phosphatase (PAP), prostate-specific antigen (PSA), PT1-1, β-catenin, PRAME, Telomerase, FAK, cyclin D1protein, NOEY2, EGF-R, SART-1, CAPB, HPVE7, p15, Folate receptor CDC27, PAGE-1, and PAGE-4. Examples of suitable infectious disease-associated antigens include hepatitis B core and surface antigens (HBVc, HBVs), hepatitis C antigens, Epstein-Barr virus antigens, human immunodeficiency virus (HIV) antigens and human papilloma virus (HPV) antigens, Mycobacterium tuberculosis and Chlamydia. Examples of suitable fungal antigens include those derived from Candida albicans, Cryptococcus neoformans, Coccidoides spp., Histoplasma spp, and Aspergillus fumigatis. Examples of suitable protozoan parasitic antigens include those derived from Plasmodium spp., including P. falciparum, Trypanosoma spp., Schistosoma spp., Leishmania spp and the like.

In some preferred aspects of the invention, the class I peptide(s) are from antigens for which the immune system of a subject has developed a tolerance, i.e., a specific immunologic nonresponsiveness induced by prior exposure to an antigen.

The epitopes that may be used as wild-type sequences to which the rules of the invention are applied to construct corresponding heteroclitic analogs can be found corresponding to any class I epitope, preferably A2, A3, A24, or B7 epitopes. For any desired antigen, such as those set forth above, the motif associated with a particular class I allele can be used as a guide to determine the positions in the amino acid sequence of the antigen wherein such an epitope would reside. This determination can be done visually or, preferably, using computer technology and associated software. Thus, for example, by recognition of the A3 supermotif as containing, for example, valine in position 2 and arginine at the C-terminus, the amino acid sequence of any desired antigen can be surveyed for epitopes bearing this motif. That epitope can then be modified according to the rules set forth in the present invention to obtain the desired analogs.

Thus, the invention relates to a method of producing an analog of a MHC class I epitope, wherein the analog has enhanced immunogenicity compared to the epitope, comprising: (a) identifying a MHC class I epitope comprising a formula (A), wherein: formula (A) is Rn-R2-R3-R4-R5-R6-R7- . . . Rx, Rn is the N-terminal amino acid, Rx is the C-terminal amino acid, x=8-11 such that Rx can be from the eighth to the eleventh amino acid residue from Rn, R2 and Rx are primary anchor residues of a motif or supermotif; and (b) producing an analog comprising a formula (B) identical to said formula (A) except for one or more conservative, semi-conservative, or non-conservative amino acid substitutions at R3 and/or R4 and/or R5 and/or R6 and/or R7, provided that, in some embodiments, said one or more substitutions is not of a primary anchor residue.

The supermotif may be A1, wherein R2 is a primary anchor residue and is either T, I, L, V, M or S, and Rx is either F, W, or Y. The motif may be an A1 motif (e.g. an A*0101 motif, etc., see “Class I Motifs” and Tables 3-5).

The supermotif may be A2, wherein R2 is a primary anchor residue and is either L, I, V, M, A, T, or Q, and Rx is I, V, M, A, T, or L. The motif may be an A2 motif (e.g. an A*0202 motif, etc., see “Class I Motifs” and Tables 3-5).

The supermotif may be A2.1, wherein R2 is a primary anchor and is either L, M, V, Q, I, A, or T, and Rx is either V, L, I, M, A, or T. The motif may be an A2.1 motif (e.g. an A*0201 motif, etc., see “Class I Motifs” and Tables 3-5).

The supermotif may be A3, wherein R2 is a primary anchor residue and is V, S, M, A, T, L, or I, and Rx is R or K. The motif may be an A3 motif (e.g. an A*0301 motif, etc., see “Class I Motifs” and Tables 3-5).

The supermotif may be A24, wherein R2 is a primary anchor residue and is Y, F, W, V, L, M, or T, and Rx is F, Y, W, L, or M. The motif may be an A24 motif (e.g. an A*2402 motif, etc., see “Class I Motifs” and Tables 3-5).

The supermotif may be B7, wherein R2 is a primary anchor residue and is P and Rx is either V, I, L, F, M, W, Y, or A. The motif may be a B7 motif (e.g. an B*0702 motif, etc., see “Class I Motifs” and Tables 3-5).

In preferred embodiments, the supermotif or motif is A3, and formula (B) is identical to formula (A) except that R4 is substituted with a conservative or a non-conservative amino acid. In preferred embodiments, R4 is substituted with P or I. In preferred embodiments, R4 of formula (A) is G and formula (B) is identical to (A) except that R4 is substituted with a conservative or a non-conservative amino acid. In preferred embodiments, R4 of formula (A) is G and formula (B) is identical to (A) except that R4 is P or I. In preferred embodiments, the epitope is CEA.61 and the analog is selected from SEQ ID NO: 11 and 12.

In preferred embodiments, the supermotif or motif is A3, and formula (B) is identical to formula (A) except that R7 is substituted with a conservative, a semi-conservative, or a non-conservative amino acid. In preferred embodiments, R7 is substituted with L, M, L D, G, C, or N. In preferred embodiments, R7 of formula (A) is W and formula (B) is identical to (A) except that R7 is substituted with a conservative, a semi-conservative, or a non-conservative amino acid. In preferred embodiments, R7 of formula (A) is W and formula (B) is identical to (A) except that R7 is L, M, L D, G, C, or N. In preferred embodiments, the epitope is CEA.61 and the analog is selected from SEQ ID NOs: 13-19.

In preferred embodiments, the supermotif or motif is A24, and formula (B) is identical to formula (A) except that R3 is substituted with a conservative amino acid. In preferred embodiments, R3 is substituted with I. In preferred embodiments, R3 of formula (A) is L and formula (B) is identical to (A) except that R3 is substituted with a conservative amino acid. In preferred embodiments, R3 of formula (A) is L and formula (B) is identical to (A) except that R3 is I. In preferred embodiments, the epitope is MAGE2.156 and the analog is SEQ ID NO:21.

In preferred embodiments, the supermotif or motif is A24, and formula (B) is identical to formula (A) except that R4 is substituted with a conservative or a non-conservative amino acid. In preferred embodiments, R4 is substituted with E or L. In preferred embodiments, R4 of formula (A) is Q and formula (B) is identical to (A) except that R4 is substituted with a conservative or a non-conservative amino acid. In preferred embodiments, R4 of formula (A) is Q and formula (B) is identical to (A) except that R4 is E or L. In preferred embodiments, the epitope is MAGE2.156 and the analog is selected from SEQ ID NOs:22 and 23.

In preferred embodiments, the supermotif or motif is A24, and formula (B) is identical to formula (A) except that R6 is substituted with a conservative amino acid. In preferred embodiments, R6 is substituted with M or L. In preferred embodiments, R6 of formula (A) is V and formula (B) is identical to (A) except that R6 is substituted with a conservative amino acid. In preferred embodiments, R6 of formula (A) is V and formula (B) is identical to (A) except that R6 is M or L. In preferred embodiments, the epitope is MAGE2.156 and the analog is selected from SEQ ID NOs:24 and 25.

Thus, the invention includes methods of producing the analogs above, and analog polypeptides comprising or consisting of each of the analogs above, and polynucleotides encoding each of said analogs and analog polypeptides, and also includes the analogs, analog polypeptides, and analog polynucleotides, themselves, as is further described below.

4. Analog Polypeptides and Analog Polynucleotides and Methods of Preparation

As noted above, “analog polypeptides” comprise or consist of an analog and are also part of the invention. Preferred analogs are set forth in the section above.

The analog polypeptide may contain 9-20 amino acids, preferably 9-16, more preferably 9-15, but may also contain only a total of 9, 10, 11, 12, 13, 14, or 15 amino acids. In certain embodiments, the polypeptide may contain not more than 250 amino acids, not more than 225 amino acids, not more than 200 amino acids, not more than 175 amino acids, not more than 150 amino acids, not more than 125 amino acids, not more than 100 amino acids, not more than 75 amino acids, not more than 50 amino acids, not more than 40 amino acids, not more than 35 amino acids, not more than 30 amino acids, not more than 25 amino acids, not more than 20 amino acids, not more than 15 amino acids, or 14, 13, 12, 11, 10, 9 or 8 amino acids.

In other embodiments, the analog polypeptide may contain at least 9-20 amino acids, preferably at least 9-16, more preferably at least 9-15, but may also contain a total of at least 9, at least 10, at least 11, at least 12, at least 13, at least 14, or at least 15 amino acids. In certain embodiments, the polypeptide may contain at least 250 amino acids, at least 225 amino acids, at least 200 amino acids, at least 175 amino acids, at least 150 amino acids, at least 125 amino acids, at least 100 amino acids, at least 75 amino acids, at least 50 amino acids, at least 40 amino acids, at least 35 amino acids, at least 30 amino acids, at least 25 amino acids, at least 20 amino acids, at least 15 amino acids, or at least 14, at least 13, at least 12, at least 11, at least 10, at least 9 or at least 8 amino acids.

When possible, it may be desirable to optimize analog polypeptides of the invention to a length of about 8 to about 13 amino acid residues (i.e., 8, 9, 10, 11, 12, or 13), often 8 to 11, preferably 9 to 10. Preferably, the analog polypeptides are commensurate in size with endogenously processed pathogen-derived epitopes or tumor cell epitopes that bind to the relevant HLA molecules (e.g., HLA-A2, -A3, -A24, or -B7), however, the identification and preparation of polypeptides that comprise analogs of the invention can also be carried out using other techniques described herein.

The analog polypeptide may comprise or consist of full-length antigen (e.g. CEA or MAGE2), or a fragment thereof, which comprises a heteroclitic analog. Fragments of full-length antigens may be fragments from about residue 1-20, 21-40, 41-60, 61-80, 81-100, or 101 to the C-terminus of the antigen CEA or MAGE2 (e.g., SEQ ID NOs:68 and 69) (Table 9). Moreover, fragments can be about 8, 9, 15, 20, 30, 40, 50, 60, 70, 80, 90, 100, 125, 150, 175, 200 or 250 amino acids in length. In this context “about” includes the particularly recited ranges or lengths, larger or smaller by several (5, 4, 3, 2, or 1) amino acids, at either extreme or at both extremes. In certain embodiments, there is a limitation on the length of a polypeptide of the invention of, for example, not more than 40 amino acids, not more than 35 amino acids, not more than 30 amino acids, not more than 25 amino acids, 20 amino acids, 15 amino acids, or 14, 13, 12, 11, 10, 9 or 8 amino acids. Thus, an analog polypeptide may comprise one or more fragments of a full-length antigen, wherein the fragment comprises an analog.

Analog polypeptides may comprise or consist of fragments of CEA selected from the group consisting of: (a) amino acids 61-69 of SEQ ID NO:68; (b) amino acids 61-70, 61-71, 61-72, 61-73, 61-74, 61-75, 61-76, 61-77, 61-78, 61-79, 61-80, 61-81, 61-82, 61-83, 61-84, 61-85, 61-86, 61-87, 61-88, 61-89, 61-90, 61-91, 61-92, 61-93, 61-94, 61-95, 61-96, 61-97, 61-98, 61-99, 61-100, 61-101, 61-102, 61-103, 61-104, 61-105, 61-106, 61-107, 61-108, 61-109, 61-110, 61-111, 61-112, 61-113, 61-114, 61-115, 61-116, 61-117, 61-118, 61-119, 61-120, 61-121, 61-122, 61-123, 61-124, 61-125, 61-126, 61-127, 61-128, 61-129, 61-130, 61-131, 61-132, 61-133, 61-134, 61-135, 61-136, 61-137, 61-138, 61-139, 61-140, 61-141, 61-142, 61-143, 61-144, 61-145, 61-146, 61-147, 61-148, 61-149, 61-150, 61-151, 61-152, 61-153, 61-154, 61-155, 61-156, 61-157, 61-158, 61-159, 61-160, 61-161, 61-162, 61-163, 61-164, 61-165, 61-166, 61-167, 61-168, 61-169, 61-170, 61-171, 61-172, 61-173, 61-174, 61-175, 61-176, 61-177, 61-178, 61-179, 61-180, 61-181, 61-182, 61-183, 61-184, 61-185, 61-186, 61-187, 61-188, 61-189, 61-190, 61-191, 61-192, 61-193, 61-194, 61-195, 61-196, 61-197, 61-198, 61-199, 61-200, 61-201, 61-202, 61-203, 61-204, 61-205, 61-206, 61-207, 61-208, 61-209, 61-210, 61-211, 61-212, 61-213, 61-214, 61-215, 61-216, 61-217, 61-218, 61-219, 61-220, 61-221, 61-222, 61-223, 61-224, 61-225, 61-226, 61-227, 61-228, 61-229, 61-230, 61-231, 61-232, 61-233, 61-234, 61-235, 61-236, 61-237, 61-238, 61-239, 61-240, 61-241, 61-242, 61-243, 61-244, 61-245, 61-246, 61-247, 61-248, 61-249, 61-250, 61-251, 61-252, 61-253, 61-254, 61-255, 61-256, 61-257, 61-258, 61-259, 61-260, 61-261, 61-262, 61-263, 61-264, 61-265, 61-266, 61-267, 61-268, 61-269, 61-270, 61-271, 61-272, 61-273, 61-274, 61-275, 61-276, 61-277, 61-278, 61-279, 61-280, 61-281, 61-282, 61-283, 61-284, 61-285, 61-286, 61-287, 61-288, 61-289, 61-290, 61-291, 61-292, 61-293, 61-294, 61-295, 61-296, 61-297, 61-298, 61-299, 61-300, 61-301, 61-302, 61-303, 61-304, 61-305, 61-306, 61-307, 61-308, 61-309, 61-310, 61-311, 61-312, 61-313, 61-314, 61-315, 61-316, 61-317, 61-318, 61-319, 61-320, 61-321, 61-322, 61-323, 61-324, 61-325, 61-326, 61-327, 61-328, 61-329, 61-330, 61-331, 61-332, 61-333, 61-334, 61-335, 61-336, 61-337, 61-338, 61-339, 61-340, 61-341, 61-342, 61-343, 61-344, 61-345, 61-346, 61-347, 61-348, 61-349, 61-350, 61-351, 61-352, 61-353, 61-354, 61-355, 61-356, 61-357, 61-358, 61-359, 61-360, 61-361, 61-362, 61-363, 61-364, 61-365, 61-366, 61-367, 61-368, 61-369, 61-370, 61-371, 61-372, 61-373, 61-374, 61-375, 61-376, 61-377, 61-378, 61-379, 61-380, 61-381, 61-382, 61-383, 61-384, 61-385, 61-386, 61-387, 61-388, 61-389, 61-390, 61-391, 61-392, 61-393, 61-394, 61-395, 61-396, 61-397, 61-398, 61-399, 61-400, 61-401, 61-402, 61-403, 61-404, 61-405, 61-406, 61-407, 61-408, 61-409, 61-410, 61-411, 61-412, 61-413, 61-414, 61-415, 61-416, 61-417, 61-418, 61-419, 61-420, 61-421, 61-422, 61-423, 61-424, 61-425, 61-426, 61-427, 61-428, 61-429, 61-430, 61-431, 61-432, 61-433, 61-434, 61-435, 61-436, 61-437, 61-438, 61-439, 61-440, 61-441, 61-442, 61-443, 61-444, 61-445, 61-446, 61-447, 61-448, 61-449, 61-450, 61-451, 61-452, 61-453, 61-454, 61-455, 61-456, 61-457, 61-458, 61-459, 61-460, 61-461, 61-462, 61-463, 61-464, 61-465, 61-466, 61-467, 61-468, 61-469, 61-470, 61-471, 61-472, 61-473, 61-474, 61-475, 61-476, 61-477, 61-478, 61-479, 61-480, 61-481, 61-482, 61-483, 61-484, 61-485, 61-486, 61-487, 61-488, 61-489, 61-490, 61-491, 61-492, 61-493, 61-494, 61-495, 61-496, 61-497, 61-498, 61-499, 61-500, 61-501, 61-502, 61-503, 61-504, 61-505, 61-506, 61-507, 61-508, 61-509, 61-510, 61-511, 61-512, 61-513, 61-514, 61-515, 61-516, 61-517, 61-518, 61-519, 61-520, 61-521, 61-522, 61-523, 61-524, 61-525, 61-526, 61-527, 61-528, 61-529, 61-530, 61-531, 61-532, 61-533, 61-534, 61-535, 61-536, 61-537, 61-538, 61-539, 61-540, 61-541, 61-542, 61-543, 61-544, 61-545, 61-546, 61-547, 61-548, 61-549, 61-550, 61-551, 61-552, 61-553, 61-554, 61-555, 61-556, 61-557, 61-558, 61-559, 61-560, 61-561, 61-562, 61-563, 61-564, 61-565, 61-566, 61-567, 61-568, 61-569, 61-570, 61-571, 61-572, 61-573, 61-574, 61-575, 61-576, 61-577, 61-578, 61-579, 61-580, 61-581, 61-582, 61-583, 61-584, 61-585, 61-586, 61-587, 61-588, 61-589, 61-590, 61-591, 61-592, 61-593, 61-594, 61-595, 61-596, 61-597, 61-598, 61-599, 61-600, 61-601, 61-602, 61-603, 61-604, 61-605, 61-606, 61-607, 61-608, 61-609, 61-610, 61-611, 61-612, 61-613, 61-614, 61-615, 61-616, 61-617, 61-618, 61-619, 61-620, 61-621, 61-622, 61-623, 61-624, 61-625, 61-626, 61-627, 61-628, 61-629, 61-630, 61-631, 61-632, 61-633, 61-634, 61-635, 61-636, 61-637, 61-638, 61-639, 61-640, 61-641, 61-642, 61-643, 61-644, 61-645, 61-646, 61-647, 61-648, 61-649, 61-650, 61-651, 61-652, 61-653, 61-654, 61-655, 61-656, 61-657, 61-658, 61-659, 61-660, 61-661, 61-662, 61-663, 61-664, 61-665, 61-666, 61-667, 61-668, 61-669, 61-670, 61-671, 61-672, 61-673, 61-674, 61-675, 61-676, 61-677, 61-678, 61-679, 61-680, 61-681, 61-682, 61-683, 61-684, 61-685, 61-686, 61-687, 61-688, 61-689, 61-690, 61-691, 61-692, 61-693, 61-694, 61-695, 61-696, 61-697, 61-698, 61-699, 61-700, 61-701, and 61-702 of SEQ ID NO:68; (c) amino acids 1-69, 2-69, 3-69, 4-69, 5-69, 6-69, 7-69, 8-69, 9-69, 10-69, 11-69, 12-69, 13-69, 14-69, 15-69, 16-69, 17-69, 18-69, 19-69, 20-69, 21-69, 22-69, 23-69, 24-69, 25-69, 26-69, 27-69, 28-69, 29-69, 30-69, 31-69, 32-69, 33-69, 34-69, 35-69, 36-69, 37-69, 38-69, 39-69, 40-69, 41-69, 42-69, 43-69, 44-69, 45-69, 46-69, 47-69, 48-69, 49-69, 50-69, 51-69, 52-69, 53-69, 54-69, 55-69, 56-69, 57-69, 58-69, 59-69, 60-69, of SEQ ID NO:68. Such fragments may, for example, comprise at least one CEA.61 analog of Table 6 (SEQ ID NOs: 11-19) or may be fused to at least one CEA.61 analog of Table 6 (SEQ ID NOs: 11-19).

Analog polypeptides may comprise or consist of fragments of MAGE2 selected from the group consisting of: (a) amino acids 157-163 of SEQ ID NO:69; (b) 1-163, 2-163, 3-163, 4-163, 5-163, 6-163, 7-163, 8-163, 9-163, 10-163, 11-163, 12-163, 13-163, 14-163, 15-163, 16-163, 17-163, 18-163, 19-163, 20-163, 21-163, 22-163, 23-163, 24-163, 25-163, 26-163, 27-163, 28-163, 29-163, 30-163, 31-163, 32-163, 33-163, 34-163, 35-163, 36-163, 37-163, 38-163, 39-163, 40-163, 41-163, 42-163, 43-163, 44-163, 45-163, 46-163, 47-163, 48-163, 49-163, 50-163, 51-163, 52-163, 53-163, 54-163, 55-163, 56-163, 57-163, 58-163, 59-163, 60-163, 61-163, 62-163, 63-163, 64-163, 65-163, 66-163, 67-163, 68-163, 69-163, 70-163, 71-163, 72-163, 73-163, 74-163, 75-163, 76-163, 77-163, 78-163, 79-163, 80-163, 81-163, 82-163, 83-163, 84-163, 85-163, 86-163, 87-163, 88-163, 89-163, 90-163, 91-163, 92-163, 93-163, 94-163, 95-163, 96-163, 97-163, 98-163, 99-163, 100-163, 101-163, 102-163, 103-163, 104-163, 105-163, 106-163, 107-163, 108-163, 109-163, 110-163, 111-163, 112-163, 113-163, 114-163, 115-163, 116-163, 117-163, 118-163, 119-163, 120-163, 121-163, 122-163, 123-163, 124-163, 125-163, 126-163, 127-163, 128-163, 129-163, 130-163, 131-163, 132-163, 133-163, 134-163, 135-163, 136-163, 137-163, 138-163, 139-163, 140-163, 141-163, 142-163, 143-163, 144-163, 145-163, 146-163, 147-163, 148-163, 149-163, 150-163, 151-163, 152-163, 153-163, 154-163, 155-163, 156-163; and (c) amino acids 157-164, 165, 157-166, 157-167, 157-168, 157-169, 157-170, 157-171, 157-172, 157-173, 157-174, 157-175, 157-176, 157-177, 157-178, 157-179, 157-180, 157-181, 157-182, 157-183, 157-184, 157-185, 157-186, 157-187, 157-188, 157-189, 157-190, 157-191, 157-192, 157-193, 157-194, 157-195, 157-196, 157-197, 157-198, 157-199, 157-200, 157-201, 157-202, 157-203, 157-204, 157-205, 157-206, 157-207, 157-208, 157-209, 157-210, 157-211, 157-212, 157-213, 157-214, 157-215, 157-216, 157-217, 157-218, 157-219, 157-220, 157-221, 157-222, 157-223, 157-224, 157-225, 157-226, 157-227, 157-228, 157-229, 157-230, 157-231, 157-232, 157-233, 157-234, 157-235, 157-236, 157-237, 157-238, 157-239, 157-240, 157-241, 157-242, 157-243, 157-244, 157-245, 157-246, 157-247, 157-248, 157-249, 157-250, 157-251, 157-252, 157-253, 157-254, 157-255, 157-256, 157-257, 157-258, 157-259, 157-260, 157-261, 157-262, 157-263, 157-264, 157-265, 157-266, 157-267, 157-268, 157-269, 157-270, 157-271, 157-272, 157-273, 157-274, 157-275, 157-276, 157-277, 157-278, 157-279, 157-280, 157-281, 157-282, 157-283, 157-284, 157-285, 157-286, 157-287, 157-288, 157-289, 157-290, 157-291, 157-292, 157-293, 157-294, 157-295, 157-296, 157-297, 157-298, 157-299, 157-300, 157-301, 157-302, 157-303, 157-304, 157-305, 157-306, 157-307, 157-308, 157-309, 157-310, 157-311, 157-312, 157-313, 157-314 of SEQ ID NO:69. Such fragments may, for example, comprise at least one MAGE2.156 analog of Table 6 (SEQ ID NOs:21-25) or may be fused to at least one CEA.61 analog of Table 6 (SEQ ID NOs:21-25).

In some embodiments, it is preferred to identify regions (“epitope-rich” regions) of an antigen that contain a high concentration of class I epitopes and/or class II epitopes. Such a region is generally selected on the basis that it contains a many epitopes per amino acid length. Preferred fragments which are epitope rich regions include amino acids 600-700 of CEA (SEQ ID NO:68), and amino acids 157-282 of MAGE2 (SEQ ID NO:69). An analog polypeptide containing a fragment of a full-length antigen may comprise or consist of such a region.

It is to be appreciated that epitopes can be present in a nested or overlapping manner, e.g., a 10 amino acid long fragment could contain two 9 amino acid long epitopes and one 10 amino acid long epitope; upon intracellular processing, each epitope can be exposed and bound by an HLA molecule upon administration of such a fragment. At least one of such epitopes is modified according to the invention to become an analog. This larger, preferably multi-epitopic, polypeptide comprising an analog can be generated synthetically, recombinantly, or via cleavage from the native source.

The analog polypeptide may also be a fusion protein, such as a homopolymer of one analog or a heteropolymer which contains at least two analogs or which contains an analog in combination with one or more CTL and/or HTL epitopes. In some embodiments, polypeptides of the invention comprise multiple analogs, an analog and multiple epitopes, or multiple analogs plus multiple epitopes, such as a polyepitopic analog polypeptide.

Analog polypeptides may comprise a first analog and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, or 150 other (different) (e.g., second, third, fourth, fifth, . . . 151^{th}) analogs and/or an analog and at least 1, 2, 3, 4, 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, or 150 (e.g., first, second, third, fourth, fifth, . . . 150^{th}) CTL and/or HTL epitopes.

Such additional analogs and/or epitopes may be from the same antigen as the first analog or may be from a different antigen. Thus, for example, if the first analog is from the A3 epitope CEA.61 (e.g., the analog is one of SEQ ID NOs:11-19), the additional analog may be a different analog from CEA.61, or may be an analog from CEA.691 (e.g., one or more of SEQ ID NOs:2-3), or any other analog from CEA. Likewise, the additional epitope may be any CTL or HTL epitope from CEA, preferably one of those from Tables 13-15 and 18. As another example, if the analog is from the A24 epitope MAGE2.156 (e.g., the analog is one of SEQ ID NOs:21-25), the additional analog may be a different analog from MAGE2.156 or may be an analog from MAGE2.157, and the additional epitope may be any epitope from MAGE2. Examples of preferred epitopes from MAGE2 are those listed in Tables 13-15 and 18. The additional analog may be a primary anchor analog, for example, those disclosed in WO 01/42270, published 14 Jun. 2001 or listed in Tables 13-15 and 18.

Alternatively, for example, if the first analog is from CEA.61 (e.g., one of SEQ ID NOs:11-19), the additional analog or epitope (CTL and/or HTL) may be from a non-CEA antigen such as a different tumor-associated antigen and/or an infectious disease antigen and/or a protozoan parasitic antigen and/or a fungal antigen.

Tumor-associated antigens include prostate specific antigens (PSA), melanoma antigens MAGE-1, MAGE-2, MAGE-3, MAGE-11, MAGE-A10, as well as BAGE, GAGE, RAGE, MAGE-C1, LAGE-1, CAG-3, DAM, MUC1, MUC2, MUC18, NY-ESO-1, MUM-1, CDK4, BRCA2, NY-LU-1, NY-LU-7, NY-LU-12, CASP8, RAS, KIAA-2-5, SCCs, p53, p73, CEA, Her 2/neu, Melan-A, gp100, tyrosinase, TRP2, gp75/TRP1, kallikrein, prostate-specific membrane antigen (PSM), prostatic acid phosphatase (PAP), prostate-specific antigen (PSA), PT1-1, β-catenin, PRAME, Telomerase, FAK, cyclin D1 protein, NOEY2, EGF-R, SART-1, CAPB, HPVE7, p15, Folate receptor CDC27, PAGE-1, and PAGE-4. See Table 12. Infectious disease-associated antigens include hepatitis B core and surface antigens (HBVc, HBVs), hepatitis C antigens, Epstein-Barr virus antigens, human immunodeficiency virus (HIV) antigens and human papilloma virus (HPV) antigens, Mycobacterium tuberculosis and Chlamydia. Fungal antigens include those derived from Candida albicans, Cryptococcus neoformans, Coccidoides spp., Histoplasma spp, and Aspergillus fumigatis. Protozoan parasitic antigens include those derived from Plasmodium spp., including P. falciparum, Trypanosoma spp., Schistosoma spp., Leishmania spp and the like.

Alternatively, an analog polypeptide may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, or 150 copies of the same analog, e.g., a homopolymer.

One or more of the analogs and/or epitopes can be modified, e.g., by addition of a surface active material, e.g. a lipid, or chemically modified, e.g., acetylation, etc. Moreover, bonds in the polypeptide can be other than peptide bonds, e.g., covalent bonds, ester or ether bonds, disulfide bonds, hydrogen bonds, ionic bonds, etc.

Analog polypeptides may comprise carriers such as those well known in the art, e.g., thyroglobulin, albumins such as human serum albumin, tetanus toxoid, polyamino acids such as poly L-lysine, poly L-glutamic acid, influenza, hepatitis B virus core protein, and the like. See, e.g., Tables 10-11.

In some embodiments, the analog polypeptides may comprise components that induce or facilitate neutralizing antibody and or helper T cell responses to the target antigen. A preferred embodiment of such a polypeptide comprises a class II epitope such as a pan-DR binding epitope (see “T Helper Epitopes” section, below). A preferred pan-DR binding epitope is the PADRE® (Epimmune, San Diego, Calif.) molecule (described, for example, in U.S. Pat. No. 5,736,142).

Analog polypeptides may comprise one or more spacers or linkers. When present, the spacer will usually be at least one or two residues, more usually three to six residues and sometimes 10 or more residues, e.g., 3, 4, 5, 6, 7, 8, 9, or 10, or even more residues. Such spacers or linkers may comprise Ala, Arg, Asn, Asp, Cys, Gln, Gly, Glu, His, Ile, Leu, Lys, Met, Phe, Pro, Ser, Thr, Tyr, Trp, Val, amino acid mimetics, and other unnatural amino acids such as those described below. Spacers or linkers may provide for ease of linking peptides one to another, for linking epitopes and/or analogs to one another, for linking epitopes and/or analogs to CTL and/or HTL epitopes, for coupling to a non-analoh polypeptide such as a carrier or larger peptide, for modifying the physical or chemical properties of the analog polypeptide, or the like. Amino acids such as Tyr, Cys, Lys, Glu or Asp, or the like, can be introduced at the C- and/or N-terminus of the polypeptide and/or can be introduced internally. The spacer is typically comprised of one or more relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need may be composed of the same residues or may be composed of one or more different residues and thus may be a homo- or hetero-oligomer. Thus, the spacer may contain more than one Ala residues or more than one Gly residues, or may contain both Ala and Gly residues.

Spacers may be at the N-terminus or C-terminus of an analog polypeptide, or may be internal such that they link or join analogs, CTL epitopes, HTL epitopes, carriers, amino acids, and antigenic fragments one to the other. A spacer flanking a class I HLA epitope in a multi-epitope polypeptide is preferably between one and about eight amino acids in length. A spacer flanking a class II HLA epitope in a multi-epitope polypeptide is preferably greater than five, six, seven, or more amino acids in length, and more preferably five or six amino acids in length.

The number of spacers in a polypeptide, the number of amino acids in a spacer, and the amino acid composition of a spacer can be selected to optimize epitope processing and/or minimize junctional epitopes. It is preferred that spacers are selected by concomitantly optimizing epitope processing and junctional motifs. Suitable amino acids for optimizing epitope processing are described WO 01/47541. Also, suitable amino acid spacing for minimizing the number of junctional epitopes in a construct are described WO 01/47541 for class I and class II HLAs. For example, spacers flanking class II HLA epitopes preferably include G, P, and/or N residues as these are not generally known to be primary anchor residues (see, e.g., PCT/US00/19774). A particularly preferred spacer for flanking a class II HLA epitope includes alternating G and P residues, for example, (GP)ₙ, (PG)ₙ, (GP)ₙG, (PG)ₙP, and so forth, where n is an integer between one and ten, preferably two or about two, and where a specific example of such a spacer is GPGPG. A preferred spacer, particularly for class I HLA epitopes, comprises one, two, three or more consecutive alanine (A) residues.

In some multi-epitope polypeptides, it is sufficient that each spacer comprise the same amino acid sequence. In multi-epitope polynucleotides having two spacer nucleic acids encoding the same amino acid sequence, the spacer nucleic acids encoding those spacers may have the same or different nucleotide sequences, where different nucleotide sequences may be preferred to decrease the likelihood of unintended recombination events when the multi-epitope polynucleotide is inserted into cells.

In other multi-epitope polypeptides, one or more of the spacers may comprise different amino acid sequences. While many of the spacers may have the same amino acid sequence in a multi-epitope polypeptide, one, two, three, four, five or more spacers may have different amino acid sequences, and it is possible that all of the spacers in a multi-epitope polypeptide have different amino acid sequences. Likewise, while many of the spacer nucleic acids may encode the same amino acid sequence in a multi-epitope polynucleotide, one, two, three, four, five or more spacer nucleic acids may encode different amino acid sequences, and it is possible that all of the spacer nucleic acids in a multi-epitope polynucleotide encode different amino acid sequences. Spacer nucleic acids may be optimized with respect to the epitope nucleic acids they flank by determining whether a spacer sequence will maximize epitope processing and/or minimize junctional epitopes, as described WO 01/47541. Computer assisted methods and in vitro and in vivo laboratory methods for determining whether a construct is optimized for epitope processing and junctional motifs are described WO 01/47541.

Analog polypeptides of the present invention may contain substitutions to modify the physical property (e.g., stability or solubility) of the resulting polypeptide. For example, analog polypeptides may be modified by the substitution of a cysteine (C) with α-amino butyric acid. Due to its chemical nature, cysteine has the propensity to form disulfide bridges and sufficiently alter the peptide structurally so as to reduce binding capacity. Substituting α-amino butyric acid for C not only alleviates this problem, but actually improves binding and crossbinding capability in certain instances. Substitution of cysteine with α-amino butyric acid may occur at any residue of a peptide epitope, i.e. at either anchor or non-anchor positions.

The analog polypeptides can comprise artificial amino acids and/or chemical modifications such as addition of a surface active molecule, e.g., lipidation; acetylation, glycosylation, biotinylation, phosphorylation etc.

Modified polypeptides that have various amino acid mimetics or unnatural amino acids are particularly useful, as they tend to manifest increased stability in vivo. Such analog polypeptides may also possess improved shelf-life or manufacturing properties. More specifically, non-critical amino acids need not be limited to those naturally occurring in proteins, such as L-α-amino acids, or their D-isomers, but may include non-natural amino acids as well, such as amino acids mimetics, e.g. D- or L-naphylalanine; D- or L-phenylglycine; D- or L-2-thieneylalanine; D- or L-1,-2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-ρ-fluorophenylalanine; D- or L-ρ-biphenylphenylalanine; D- or L-ρ-methoxybiphenylphenylalanine; D- or L-2-indole(alkyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Polypeptide stability can be assayed in a number of ways. For instance, peptidases and various biological media, such as human plasma and serum, have been used to test stability. See, e.g., Verhoef, et al., Eur. J. Drug Metab. Pharmacokinetics 11:291 (1986). Half-life of the peptides of the present invention is conveniently determined using a 25% human serum (v/v) assay. The protocol is generally as follows: Pooled human serum (Type AB, non-heat inactivated) is delipidated by centrifugation before use. The serum is then diluted to 25% with RPMI-1640 or another suitable tissue culture medium. At predetermined time intervals, a small amount of reaction solution is removed and added to either 6% aqueous trichloroacetic acid (TCA) or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The analog polypeptides in accordance with the invention can be either in their neutral (uncharged) forms or in forms which are salts. The polypeptides in accordance with the invention are either free of modifications such as glycosylation, side chain oxidation, or phosphorylation; or they contain these modifications, subject to the condition that modifications do not destroy the biological activity of the polypeptides or analogs as described herein.

The polypeptides of the invention can be prepared in a wide variety of ways. Polypeptides in accordance with the invention can be prepared synthetically, by recombinant DNA technology or chemical synthesis, or from natural sources such as native tumors or pathogenic organisms. Analog polypeptides may be synthesized individually or as polyepitopic polypeptides. Although the analog polypeptide will preferably be substantially free of other naturally occurring host cell proteins and fragments thereof, in some embodiments the analog polypeptides may be synthetically conjugated to native fragments or particles.

For those of relatively short size, the analog polypeptides can be synthesized in solution or on a solid support in accordance with conventional techniques. Various automatic synthesizers are commercially available and can be used in accordance with known protocols. (See, for example, Stewart & Young, SOLID PHASE PEPTIDE SYNTHESIS, 2D. ED., Pierce Chemical Co., 1984). Further, individual analogs and epitopes can be joined using chemical ligation to produce larger analog polypeptides that are still within the bounds of the invention.

Alternatively, recombinant DNA technology can be employed wherein a nucleotide sequence which encodes an analog polypeptide is inserted into an expression vector, transformed or transfected into an appropriate host cell and cultivated under conditions suitable for expression. These procedures are generally known in the art, as described generally in Sambrook, et al., MOLECULAR CLONING, A LABORATORY MANUAL, Cold Spring Harbor Press, Cold Spring Harbor, N.Y. (1989). Thus, recombinant polypeptides of the invention can be used to present the appropriate T cell analog.

The nucleotide coding sequence for analog polypeptides can be synthesized by chemical techniques, for example, the phosphotriester method of Matteucci, et al., J. Am. Chem. Soc. 103:3185 (1981). Analog polynucleotides can be made simply by substituting the appropriate and desired nucleic acid base(s) for those that encode the wild type epitope. The coding sequence can then be provided with appropriate linkers and ligated into expression vectors commonly available in the art, and the vectors used to transform suitable hosts to produce the desired fusion protein. A number of such vectors and suitable host systems are now available.

For expression of the polypeptides, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast, insect or mammalian cell hosts may also be used, employing suitable vectors and control sequences.

Additionally, nucleic acids that encode one or more analog polypeptides are also part of the invention. As appreciated by one of ordinary skill in the art, various nucleic acids will encode the same polypeptide due to the redundancy of the genetic code. Each of these nucleic acids falls within the scope of the present invention. This embodiment of the invention comprises DNA or RNA, and in certain embodiments a combination of DNA and RNA. It is to be appreciated that any nucleic acid that encodes a polypeptide in accordance with the invention falls within the scope of this invention.

A preferred means of administering nucleic acids encoding the polypeptides of the invention uses minigene constructs encoding a polypeptide comprising one or multiple analogs and epitopes.

The use of analog polynucleotides such as multi-epitope minigenes is described below and in, e.g., co-pending application U.S. Ser. No. 09/311,784; Ishioka et al., J. Immunol. 162:3915-3925, 1999; An, L. and Whitton, J. L., J. Virol. 71:2292, 1997; Thomson, S. A. et al., J. Immunol. 157:822, 1996; Whitton, J. L. et al., J. Virol. 67:348, 1993; Hanke, R. et al., Vaccine 16:426, 1998. For example, a polynucleotide such as a multi-epitope DNA plasmid encoding an analog, supermotif- and/or motif-bearing epitopes (e.g., PSA, PSM, PAP, and hK2) derived from multiple regions of a TAA, a pan-DR binding peptide such as the PADRE® universal helper T cell epitope, and an endoplasmic reticulum-translocating signal sequence can be engineered. As descibed in the sections above, an analog polypeptide/polynucleotide may also comprise/encode epitopes that are derived from other TAAs.

For example, to create a DNA sequence such as a mini-gene encoding the selected analogs(s) and/or epitopes for expression in human cells, the amino acid sequences of the analog(s) and/or epitopes may be reverse translated. A human codon usage table can be used to guide the codon choice for each amino acid. See, e.g., Lathe (1985, J. Mol. Biol. 183:1-12); WO 97/3115. These analog/epitope-encoding DNA sequences may be directly adjoined, so that when translated, a continuous polypeptide sequence is created. To optimize expression and/or immunogenicity, additional elements can be incorporated into the polynucleotide (e.g., minigene) design. Examples of amino acid sequences in addition to the analog(s) that can be reverse translated and included in the polynucleotide (e.g., minigene) sequence include: HLA class I epitopes, HLA class II epitopes, a ubiquitination signal sequence, and/or an endoplasmic reticulum targeting signal. In addition, HLA presentation of CTL and HTL epitopes may be improved by including synthetic (e.g. poly-alanine) or naturally-occurring flanking sequences adjacent to the analog, CTL or HTL epitopes; these larger peptides comprising the analog(s) and/or epitope(s) are within the scope of the invention.

The polynucleotide (e.g., minigene) sequence may be converted to DNA by assembling oligonucleotides that encode the plus and minus strands of the polynucleotide (e.g., minigene). Overlapping oligonucleotides (30-100 bases long) may be synthesized, phosphorylated, purified and annealed under appropriate conditions using well known techniques. The ends of the oligonucleotides can be joined, for example, using T4 DNA ligase. This synthetic polynucleotide (e.g., minigene), encoding the analog polypeptide, can then be cloned into a desired expression vector.

For therapeutic or prophylactic immunization purposes, the polypeptides of the invention can also be expressed by viral or bacterial vectors. Examples of expression vectors include attenuated viral hosts, such as vaccinia or fowlpox. As an example of this approach, vaccinia virus is used as a vector to express nucleotide sequences that encode the polypeptides of the invention. Upon introduction into a host bearing a tumor, the recombinant vaccinia virus expresses the polypeptide, and thereby elicits a host CTL and/or HTL response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the polypeptides of the invention, e.g. adeno and adeno-associated virus vectors, retroviral vectors, Salmonella typhi vectors, detoxified anthrax toxin vectors, and the like, will be apparent to those skilled in the art from the description herein.

Standard regulatory sequences well known to those of skill in the art are preferably included in the vector to ensure expression in the target cells. Several vector elements are desirable: a promoter with a down-stream cloning site for analog polynucleotide (e.g., minigene) insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences.

Additional vector modifications may be desired to optimize polynucleotide (e.g., minigene) expression and immunogenicity. In some cases, introns are required for efficient gene expression, and one or more synthetic or naturally-occurring introns could be incorporated into the transcribed region of the polynucleotide (e.g., minigene). The inclusion of mRNA stabilization sequences and sequences for replication in mammalian cells may also be considered for increasing polynucleotide (e.g., minigene) expression.

Once an expression vector is selected, the polynucleotide (e.g., minigene) is cloned into the polylinker region downstream of the promoter. This plasmid is transformed into an appropriate E. coli strain, and DNA is prepared using standard techniques. The orientation and DNA sequence of the polynucleotide (e.g., minigene), as well as all other elements included in the vector, are confirmed using restriction mapping and DNA sequence analysis. Bacterial cells harboring the correct plasmid can be stored as a master cell bank and a working cell bank.

In addition, immunostimulatory sequences, such as ISSs or CpGs, appear to play a role in the immunogenicity of DNA vaccines. These sequences may be included in the vector, outside the polynucleotide (e.g., minigene) coding sequence, if desired to enhance immunogenicity.

In some embodiments, a bi-cistronic expression vector which allows production of both the polynucleotide (e.g., minigene)-encoded analog polypeptide and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF), costimulatory molecules, or for HTL responses, pan-DR binding proteins (e.g., PADRE® peptide, Epimmune, San Diego, Calif.). Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-β) may be beneficial in certain diseases.

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins supplied by QIAGEN, Inc. (Valencia, Calif.). If required, supercoiled DNA can be isolated from the open circular and linear forms using gel electrophoresis or other methods.
T Helper Epitopes

Analog polypeptides can be modified to provide desired attributes, such as improved serum half-life, or to enhance immunogenicity.

For instance, the ability of an analog polypeptide to induce CTL activity can be enhanced by linking it to or co-administering it with a sequence which contains at least one epitope that is capable of inducing a T helper cell response. The use of T helper epitopes in conjunction with CTL epitopes to enhance immunogenicity is illustrated, for example, in the co-pending applications U.S. Ser. No. 08/820,360, U.S. Ser. No. 08/197,484, and U.S. Ser. No. 08/464,234.

Although an analog or analog polypeptide can be directly linked to a T helper peptide, an analog polypeptide and an HTL epitope may be linked by a spacer or linker, such as those described in the section above. The analog polypeptide can be linked to the T helper epitope directly or via a spacer at the amino or carboxy terminus of the analog. The amino terminus of the analog polypeptide or the T helper peptide may be acylated.

In certain embodiments, the T helper peptide is one that is recognized by T helper cells present in the majority of the population. This can be accomplished by selecting amino acid sequences that bind to many, most, or all of the HLA class II molecules. These are known as “loosely HLA-restricted” or “promiscuous” T helper sequences. Examples of T helper peptides that are promiscuous include sequences from antigens such as tetanus toxoid at positions 830-843 (QYIKANSKFIGITE) (SEQ ID NO:26), Plasmodium falciparum circumsporozoite (CS) protein at positions 378-398 (DIEKKIAKMEKASSVFNVVNS) (SEQ ID NO:27), and Streptococcus 18 kD protein at positions 116 (GAVDSILGGVATYGAA) (SEQ ID NO:28). Other examples include peptides bearing a DR 1-4-7 supermotif, or either of the DR3 motifs.

Alternatively, it is possible to prepare synthetic peptides capable of stimulating T helper lymphocytes in a loosely HLA-restricted fashion, using amino acid sequences not found in nature (see, e.g., PCT publication WO 95/07707). These synthetic T helper peptides called Pan-DR-binding epitopes (e.g., PADRE® peptides, Epimmune, Inc., San Diego, Calif.) are designed to most preferrably bind most HLA-DR (human HLA class II) molecules. For instance, a pan-DR-binding epitope peptide having the formula: aKXVAAWTLKAAa, where “X” is either cyclohexylalanine (SEQ ID NO:29), phenylalanine (SEQ ID NO:30), or tyrosine (SEQ ID NO:31), and “a” is either D-alanine or L-alanine, has been found to bind to most HLA-DR alleles, and to stimulate the response of T helper lymphocytes from most individuals, regardless of their HLA type. An alternative of a pan-DR binding epitope comprises all “L” natural amino acids and can be provided in the form of nucleic acids that encode the epitope.

HTL epitopes can also be modified to alter their biological properties. For example, they can be modified to include D-amino acids to increase their resistance to proteases and thus extend their serum half life, or they can be conjugated to other molecules such as lipids, proteins, carbohydrates, and the like to increase their biological activity. For example, a T helper peptide can be conjugated to one or more palmitic acid chains at either the amino or carboxyl termini.

Selection of Analogs and Epitopes and Other Components

Preferably, the following principles are utilized when selecting an array of analogs(s) and epitope(s) for inclusion in a polyepitopic composition for use in a vaccine, or for selecting discrete epitopes to be included in a vaccine and/or to be encoded by nucleic acids such as a minigene. It is preferred that each of the following principles are balanced in order to make the selection. The multiple epitopes to be incorporated in a given vaccine composition may be, but need not be, contiguous in sequence in the native antigen from which the epitopes are derived.

1) Epitopes are selected which, upon administration, mimic immune responses that have been observed to correlate with tumor clearance. For HLA class I, this includes 3-4 epitopes that come from at least one tumor-associated antigen (TAA). For HLA class II, a similar rationale is employed; again 3-4 epitopes are selected from at least one TAA (see e.g., Rosenberg et al., Science 278:1447-1450). Epitopes from one TAA may be used in combination with epitopes from one or more additional TAAs to produce a vaccine that targets tumors with varying expression patterns of frequently expressed TAAs.

2) Epitopes are selected that have the requisite binding affinity established to be correlated with immunogenicity: for HLA class I an IC₅₀ of 500 nM or less, often 200 nM or less; and for class II an IC₅₀ of 1000 nM or less.

3) Sufficient supermotif bearing-peptides, or a sufficient array of allele-specific motif-bearing peptides, are selected to give broad population coverage. For example, it is preferable to have at least 80% population coverage. A Monte Carlo analysis, a statistical evaluation known in the art, can be employed to assess the breadth, or redundancy of, population coverage.

4) When selecting epitopes from cancer-related antigens it is often useful to select analogs because the patient may have developed tolerance to the native epitope. When selecting epitopes for infectious disease-related antigens, it is preferable to select either native or analoged epitopes.

5) Of particular relevance are epitopes referred to as “nested epitopes.” Nested epitopes occur where at least two epitopes overlap in a given peptide sequence. A nested peptide sequence can comprise both HLA class I and HLA class II epitopes. When providing nested epitopes, a general objective is to provide the greatest number of epitopes per sequence. Thus, an aspect to avoid is providing a peptide that is any longer than the amino terminus of the amino terminal epitope and the carboxyl terminus of the carboxyl terminal epitope in the peptide. When providing a multi-epitopic sequence, such as a sequence comprising nested epitopes, it is generally important to screen the sequence in order to insure that it does not have pathological or other deleterious biological properties.

6) If a polyepitopic protein is created, or when creating a polynucleotide (e.g., minigene), an objective is to generate the smallest peptide that encompasses the epitopes of interest. This principle is similar, if not the same as that employed when selecting a peptide comprising nested epitopes. However, with an artificial polyepitopic peptide, the size minimization objective is balanced against the need to integrate any spacer sequences between epitopes in the polyepitopic protein. Spacer amino acid residues can, for example, be introduced to avoid junctional epitopes (an epitope recognized by the immune system, not present in the target antigen, and only created by the man-made juxtaposition of epitopes), or to facilitate cleavage between epitopes and thereby enhance epitope presentation. Junctional epitopes are generally to be avoided because the recipient may generate an immune response to that non-native epitope. Of particular concern is a junctional epitope that is a “dominant epitope.” A dominant epitope may lead to such a zealous immune response that immune responses to other epitopes are diminished or suppressed.

A number of different approaches are available which allow simultaneous delivery of multiple epitopes. For example, nucleic acids encoding the polypeptides of the invention are a particularly useful embodiment of the invention. Analogs and epitopes for inclusion in an analog polypeptide or polynucleotide such as a minigene are preferably selected according to the guidelines set forth above.

The immunogenicity of an analog polypeptide or polynucleotide such as a multi-epitopic minigene can be tested in transgenic mice to evaluate the magnitude of CTL induction responses against the analogs/epitopes tested. Further, the immunogenicity of polypeptides encoded by analog polynucleotides in vivo can be correlated with the in vitro responses of specific CTL lines against target cells transfected with the analog polynucleotide.

Thus, these experiments can show that the analog polypeptide or polynucleotide (e.g., minigene) serves to both: (1) generate a CTL response, and (2) that the induced CTLs recognized cells expressing the encoded analog(s) and or epitope(s).

Purified plasmid DNA can be prepared for injection using a variety of formulations. The simplest of these is reconstitution of lyophilized DNA in sterile phosphate-buffered saline (PBS). This approach, known as “naked DNA,” is currently being used for intramuscular (IM) administration in clinical trials. To maximize the immunotherapeutic effects of polynucleotide (e.g., minigene) vaccines, an alternative method for formulating purified plasmid DNA may be desirable. A variety of methods have been described, and new techniques may become available. Cationic lipids, glycolipids, and fusogenic liposomes can also be used in the formulation (see, e.g., as described by WO 93/24640; Mannino & Gould-Fogerite, BioTechniques 6(7): 682 (1988); U.S. Pat No. 5,279,833; WO 91/06309; and Felgner, et al., Proc. Nat'l Acad. Sci. USA 84:7413 (1987). In addition, peptides and compounds referred to collectively as protective, interactive, non-condensing compounds (PINC) could also be complexed to purified plasmid DNA to influence variables such as stability, intramuscular dispersion, or trafficking to specific organs or cell types.

Target cell sensitization can be used as a functional assay for expression and HLA class I presentation of analog polypeptides or polynucleotide (e.g., minigene)-encoded analog(s) and/or epitope(s). For example, the plasmid DNA is introduced into a mammalian cell line that is suitable as a target for standard CTL chromium release assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 (⁵¹Cr) labeled and used as target cells for epitope-specific CTL lines; cytolysis, detected by ⁵¹Cr release, indicates both production of, and HLA presentation of, polynucleotide (e.g., minigene)-encoded CTL epitopes. Expression of HTL epitopes may be evaluated in an analogous manner using assays to assess HTL activity.

In vivo immunogenicity is a second approach for functional testing of polynucleotide (e.g., minigene) formulations. Transgenic mice expressing appropriate human HLA proteins are immunized with the DNA product. The dose and route of administration can be formulation dependent (e.g., IM for DNA in PBS, intraperitoneal (IP) for lipid-complexed DNA). Twenty-one days after immunization, splenocytes are harvested and restimulated for one week in the presence of peptides encoding each epitope being tested. Thereafter, for CTL effector cells, assays are conducted for cytolysis of peptide-loaded, ⁵¹Cr-labeled target cells using standard techniques. Lysis of target cells that were sensitized by HLA loaded with peptide epitopes, corresponding to polynucleotide (e.g., minigene)-encoded epitopes, demonstrates DNA vaccine function for in vivo induction of CTLs. Immunogenicity of HTL epitopes is evaluated in transgenic mice in an analogous manner.

Alternatively, the nucleic acids can be administered intradermally, e.g. by injection or ballistic delivery as described, for instance, in U.S. Pat. No. 5,204,253. Using this technique, particles comprised solely of DNA are administered. In a further alternative embodiment, DNA can be adhered to particles, such as gold particles.

Polynucleotide (e.g., minigene)s can also be delivered using other bacterial or viral delivery systems well known in the art, e.g., an expression construct encoding epitopes of the invention can be incorporated into a viral vector such as vaccinia.

4. Compositions

A composition of the invention may contain more than one analog polypeptides and/or analog polynucleotides of the invention and optionally another component, or a composition may contain one analog polypeptide or one polynucleotide of the invention and another component. Additional components include excipients, diluents, non-analog polypeptide (e.g., a polypeptide comprising a CTL epitope, and/or an HTL epitope such as a pan-DR binding peptide, and/or a carrier), a polynucleotide encoding such a non-analog polypeptide, a lipid, or a liposome, as well as other components described herein. There are numerous embodiments of compositions in accordance with the invention, such as a cocktail of one or more analog polypeptides and/or analog polynucleotides; one or more analogs and one or more CTL and/or HTL epitopes; and/or nucleic acids that encode such peptides or polypeptides, e.g., a minigene that encodes a polyepitopic analog polypeptide.

Compositions may comprise one or more analog polypeptides (or analog polynucleotides such as minigenes) of the invention, along with one or more other components as described above and herein. “One or more” refers to any whole unit integer from 1-150, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 , 45, 50, 55, 60, 65, 70, 75, 80, 85, 90, 95, 100, 105, 110, 115, 120, 125, 130, 135, 140, 145, or 150 analog polypeptides, analog polynucleotides, or other components.

Compositions of the invention may comprise non-analog polypeptides. Non-analog polypeptides include carriers. Carriers that can be used with compositions of the invention are well known in the art, and include, e.g., thyroglobulin, albumins such as human serum albumin, tetanus toxoid, polyamino acids such as poly L-lysine, poly L-glutamic acid, influenza, hepatitis B virus core protein, and the like. Non-analog polypeptides also include proteins that may enhance or decrease immunogenicity. Non-analog proteins that could beneficially enhance the immune response include cytokines (e.g., IL-2, IL-12, GM-CSF), cytokine-inducing molecules (e.g., LeIF), costimulatory molecules, HTL epitopes such as pan-DR binding proteins (e.g., PADRE® peptide, Epimmune, San Diego, Calif.). Helper (HTL) epitopes can be joined to intracellular targeting signals. If required, this could facilitate more efficient entry of HTL epitopes into the HLA class II pathway, thereby improving HTL induction. Non-analog proteins which may decrease the immune response include, e.g., TGF-β.

The compositions can contain a physiologically tolerable (i.e., acceptable) diluent such as water, or saline, preferably phosphate buffered saline. The compositions also typically include an adjuvant. Adjuvants such as incomplete Freund's adjuvant, aluminum phosphate, aluminum hydroxide, or alum are examples of materials well known in the art.

In some embodiments it may be desirable to include in the compositions of the invention at least one component which primes cytotoxic T lymphocytes. Lipids have been identified as agents capable of priming CTL in vivo against viral antigens. For example, palmitic acid residues can be attached to the ε- and α-amino groups of a lysine residue and then linked, e.g., via one or more linking residues such as Gly, Gly-Gly-, Ser, Ser-Ser, or the like, to an immunogenic peptide. The lipidated peptide can then be administered either directly in a micelle or particle, incorporated into a liposome, or emulsified in an adjuvant, e.g., incomplete Freund's adjuvant. A preferred composition comprises palmitic acid attached to ε- and α-amino groups of Lys, which is attached via linkage, e.g., Ser-Ser, to the amino terminus of the polypeptide.

As another example of lipid priming of CTL responses, E. coli lipoproteins, such as tripalmitoyl-S-glycerylcysteinlyseryl-serine (P₃CSS) can be used to prime virus specific CTL when covalently attached to an appropriate peptide (see, e.g., Deres, et al., Nature 342:561, 1989). Polypeptides of the invention can be coupled to P₃CSS, for example, and the lipopeptide administered to an individual to specifically prime a CTL response to the target antigen. Moreover, because the induction of neutralizing antibodies can also be primed with P₃CSS-conjugated epitopes, two such compositions can be combined to more effectively elicit both humoral and cell-mediated responses.

CTL and/or HTL peptides can also be modified by the addition of amino acids to the termini of a peptide to provide for ease of linking peptides one to another, for coupling to a carrier support or larger peptide, for modifying the physical or chemical properties of the peptide or oligopeptide, or the like. Amino acids such as tyrosine, cysteine, lysine, glutamic or aspartic acid, or the like, can be introduced at the carboxyl- or amino-terminus of the peptide or oligopeptide, particularly class I peptides. However, it is to be noted that modification at the carboxyl terminus of a CTL epitope may, in some cases, alter binding characteristics of the peptide. In addition, the peptide or oligopeptide sequences can differ from the natural sequence by being modified by terminal-NH₂ acylation, e.g., by alkanoyl (C₁-C₂₀) or thioglycolyl acetylation, terminal-carboxyl amidation, e.g., ammonia, methylamine, etc. In some instances these modifications may provide sites for inking to a support or other molecule.

Compositions can also include, for example, lipopeptides (e.g., Vitiello, A. et al., J. Clin. Invest. 95:341, 1995), polypeptides encapsulated in polyDL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995), polypeptides contained in immune stimulating complexes (ISCOMS) (see, e.g., Takahashi et al., Nature 344:873-875, 1990; Hu et al., Clin Exp Immunol. 113:235-243, 1998), multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996), polypeptides formulated as multivalent peptides; polypeptides for use in ballistic delivery systems, typically crystallized polypeptides, viral delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990), particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995), adjuvants (Warren, H. S., Vogel, F. R., and Chedid, L. A. Annu. Rev. Immunol. 4:369, 1986; Gupta, R. K. et al., Vaccine 11:293, 1993), liposomes (eddy, R. et al., J. Immunol. 148:1585, 1992; Rock, K. L., Immunol. Today 17:131, 1996), or, naked or particle absorbed cDNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993). Toxin-targeted delivery technologies, also known as receptor mediated targeting, such as those of Avant Immunotherapeutics, Inc. (Needham, Mass.) may also be used.

A further embodiment of a composition in accordance with the invention is an antigen presenting cell that comprises one or more polypeptides in accordance with the invention. The antigen presenting cell can be a “professional” antigen presenting cell, such as a dendritic cell. The antigen presenting cell can comprise the polypeptide of the invention by any means known or to be determined in the art. Such means include pulsing of dendritic cells with one or more individual analog polypeptides, by nucleic acid administration such as ballistic nucleic acid delivery or by other techniques in the art for administration of nucleic acids, including vector-based, e.g. viral vector, delivery of nucleic acids.

5. Administration of Polypeptides, Polynucleotides and Compositions

Vaccines and methods of preparing vaccines that contain an immunogenically effective amount of one or more analog polypeptides or analog polynucleotides as described herein are further embodiments of the invention. Once appropriately immunogenic polypeptides have been defined, they can be sorted and delivered by various means, herein referred to as “vaccine” compositions. The compositions described throughout this disclosure can be used as vaccines.

Such vaccine compositions can include, for example, lipopeptides (e.g.,Vitiello, A. et al., J. Clin. Invest. 95:341, 1995), peptide compositions encapsulated in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995), peptide compositions contained in immune stimulating complexes (ISCOMS) (see, e.g., Takahashi et al., Nature 344:873-875, 1990; Hu et al., Clin Exp Immunol. 113:235-243, 1998), multiple antigen peptide systems (MAPs) (see e.g., Tam, J. P., Proc. Natl. Acad. Sci. U.S.A. 85:5409-5413, 1988; Tam, J.P., J. Immunol. Methods 196:17-32, 1996), peptides formulated as multivalent peptides; peptides for use in ballistic delivery systems, typically crystallized peptides, viral delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990), particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995), adjuvants (Warren, H. S., Vogel, F. R., and Chedid, L. A. Annu. Rev. Immunol. 4:369, 1986; Gupta, R. K. et al., Vaccine 11:293, 1993), liposomes (Reddy, R. et al., J. Immunol. 148:1585, 1992; Rock, K. L., Immunol. Today 17:131, 1996), or, naked or particle absorbed cDNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993). Toxin-targeted delivery technologies, also known as receptor mediated targeting, such as those of Avant Immunotherapeutics, Inc. (Needham, Mass.) may also be used.

Vaccines of the invention include nucleic acid-mediated modalities. DNA or RNA encoding one or more of the polypeptides of the invention can also be administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the polypeptides of the invention can also be expressed by viral or bacterial vectors. Examples of expression vectors include attenuated viral hosts, such as vaccinia or fowlpox. As an example of this approach, vaccinia virus is used as a vector to express nucleotide sequences that encode the peptides of the invention. Upon introduction into a host bearing a tumor, the recombinant vaccinia virus expresses the immunogenic peptide, and thereby elicits a host CTL and/or HTL response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the polypeptides of the invention, e.g. adeno and adeno-associated virus vectors, retroviral vectors, Salmonella typhi vectors, detoxified anthrax toxin vectors, and the like, will be apparent to those skilled in the art from the description herein.

Furthermore, as described above, compositions and vaccines in accordance with the invention encompass compositions of one or more of the claimed polypeptides. An analog or polynucleotide encoding an analog can be present in a vaccine individually. Alternatively, a polypeptide comprising an analog can exist as a homopolymer comprising multiple copies of the same analog, or as a heteropolymer of various analogs and/or epitopes (CTL and/or HTL). Vaccines comprising analog polynucleotides encoding such polypeptides ate also included in the invention. Polymers have the advantage of increased immunological reaction and, where different analogs or different analogs and epitopes are used to make up the polymer, the additional ability to induce antibodies and/or CTLs that react with different antigenic determinants of the pathogenic organism or tumor-related peptide targeted for an immune response. The composition can be a naturally occurring region of an antigen or can be prepared, e.g., recombinantly or by chemical synthesis.

Carriers that can be used with vaccines of the invention are well known in the art, and include, e.g., thyroglobulin, albumins such as human serum albumin, tetanus toxoid, polyamino acids such as poly L-lysine, poly L-glutamic acid, influenza, hepatitis B virus core protein, and the like. The vaccines can contain a physiologically tolerable (i.e., acceptable) diluent such as water, or saline, preferably phosphate buffered saline. The vaccines also typically include an adjuvant. Adjuvants such as incomplete Freund's adjuvant, aluminum phosphate, aluminum hydroxide, or alum are examples of materials well known in the art. Additionally, as disclosed herein, CTL responses can be primed by conjugating polypeptides of the invention to lipids, such as tripalmitoyl-S-glycerylcysteinlyseryl-serine P₃CSS).

The polypeptides of the invention may also be administered via liposomes, which serve to target the peptides to a particular tissue, such as lymphoid tissue, or to target selectively to infected cells, as well as to increase the half-life of the peptide composition. Liposomes include emulsions, foams, micelles, insoluble monolayers, liquid crystals, phospholipid dispersions, lamellar layers and the like. In these preparations, the peptide to be delivered is incorporated as part of a liposome, alone or in conjunction with a molecule which binds to a receptor prevalent among lymphoid cells, such as monoclonal antibodies which bind to the CD45 antigen, or with other therapeutic or immunogenic compositions. Thus, liposomes either filled or decorated with a desired peptide of the invention can be directed to the site of lymphoid cells, where the liposomes then deliver the peptide compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), and U.S. Pat. Nos. 4,235,871, 4,501,728, 4,837,028, and 5,019,369.

For targeting cells of the immune system, a ligand to be incorporated into the liposome can include, e.g., antibodies or fragments thereof specific for cell surface determinants of the desired immune system cells. A liposome suspension containing a peptide may be administered intravenously, locally, topically, etc., in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

Upon immunization with a polypeptide or polynucleotide composition in accordance with the invention, via injection, aerosol, oral, transdermal, transmucosal, intrapleural, intrathecal, or other suitable routes, the immune system of the host responds to the vaccine by producing large amounts of CTLs and/or HTLs specific for the desired antigen. Consequently, the host becomes at least partially immune to later infection, or at least partially resistant to developing an ongoing chronic infection, or derives at least some therapeutic benefit when the antigen was tumor-associated.

In some embodiments, it may be desirable to combine the heteroclitic analog peptides of the invention with components that induce or facilitate neutralizing antibody and or helper T cell responses to the target antigen of interest. A preferred embodiment of such a composition comprises class I and class II epitopes in accordance with the invention. An alternative embodiment of such a composition comprises a class I and/or class II epitope in accordance with the invention, along with a pan-DR binding peptide such as the PADRE® (Epimmune, San Diego, Calif.) molecule (described, for example, in U.S. Pat. No. 5,736,142).

A vaccine of the invention can also include antigen-presenting cells (APC), such as dendritic cells (DC), as a vehicle to present peptides of the invention. Vaccine compositions can be created in vitro, following dendritic cell mobilization and harvesting, whereby loading of dendritic cells occurs in vitro. For example, dendritic cells are transfected, e.g., with a polynucleotide (e.g., minigene) in accordance with the invention, or are pulsed with peptides. The dendritic cell can then be administered to a patient to elicit immune responses in vivo.

Vaccine compositions, either DNA- or peptide-based, can also be administered in vivo in combination with dendritic cell mobilization whereby loading of dendritic cells occurs in vivo.

An embodiment of a vaccine composition in accordance with the invention comprises ex vivo administration of an analog polypeptide, preferably as part of a cocktail of epitope-bearing peptides, to PBMC, or DC isolated therefrom, from the patient's blood. A pharmaceutical compound to facilitate harvesting of DC can be used, such as Progenipoietin™ protein (Monsanto, St. Louis, Mo.) or GM-CSF/IL-4. After pulsing the DC with peptides and prior to reinfusion into patients, the DC are washed to remove unbound peptides. In this embodiment, a vaccine comprises peptide-pulsed DCs that present the pulsed peptide epitopes complexed with HLA molecules on their surfaces.

The DC can be pulsed ex vivo with a cocktail of peptides, some of which stimulate CTL response to one or more antigens of interest. Optionally, a helper T cell peptide such as a PADRE® family molecule, can be included to facilitate the CTL response.

6. Administration in Cancer Patients

The polypeptides, analog polynucleotides, compositions, and vaccines of the present invention are typically used therapeutically to treat cancer. Vaccine compositions containing the polypeptides of the invention are typically administered to a cancer patient who has a malignancy associated with expression of one or more antigens. Alternatively, vaccine compositions can be administered to an individual susceptible to, or otherwise at risk for developing cancer.

Analog polypeptides can be delivered directly or using such agents as liposomes. They can additionally be delivered using ballistic delivery, in which the polypeptides are typically in a crystalline form.

In therapeutic applications, polypeptides and/or analog polynucleotides are administered to a patient in an amount sufficient to elicit an effective CTL and/or HTL response to the tumor antigen and to cure or at least partially arrest or slow symptoms and/or complications. An amount adequate to accomplish this is defined as “therapeutically effective dose.” Amounts effective for this use will depend on, e.g., the particular composition administered, the manner of administration, the stage and severity of the disease being treated, the weight and general state of health of the patient, and the judgment of the prescribing physician.

As noted above, analog polypeptides induce immune responses when presented by HLA molecules and contacted with a CTL specific for an epitope comprised by the analog. The polypeptides (or analog polynucleotides encoding them) can be administered individually or mixtures or as compositions. The manner in which the polypeptide is contacted with the CTL is not critical to the invention. For instance, the polypeptide can be contacted with the CTL either in vivo or in vitro. If the contacting occurs in vivo, the polypeptide itself can be administered to the patient, or other vehicles, e.g., analog polynucleotides (e.g., in DNA vectors or viral vectors), liposomes and the like, can be used, as described herein.

When the polypeptide is contacted in vitro, the vaccinating agent can comprise a population of cells, e.g., peptide-pulsed dendritic cells, or TAA-specific CTLs, which have been induced by pulsing antigen-presenting cells in vitro with the polypeptide or by transfecting antigen-presenting cells with a polynucleotide (e.g., minigene) of the invention. Such a cell population is subsequently administered to a patient in a therapeutically effective dose.

For therapeutic use, administration should generally begin at the first diagnosis of cancer. This is followed by boosting doses until at least symptoms are substantially abated and for a period thereafter. The embodiment of the vaccine composition (Le., including, but not limited to embodiments such as peptide cocktails, polyepitopic polypeptides, polynucleotide (e.g., minigene)s, or TAA-specific CTLs or pulsed dendritic cells) delivered to the patient may vary according to the stage of the disease or the patient's health status. For example, a vaccine comprising TAA-specific CTLs may be more efficacious in killing tumor cells in patients with advanced disease than alternative embodiments.

Antigenic peptides are used to elicit a CTL and/or HTL response ex vivo, as well. The resulting CTL or HTL cells, can be used to treat tumors in patients that do not respond to other conventional forms of therapy, or will not respond to a therapeutic vaccine peptide or nucleic acid in accordance with the invention. Ex vivo CTL or HTL responses to a particular tumor-associated antigen are induced by incubating in tissue culture the patient's, or genetically compatible, CTL or HTL precursor cells together with a source of antigen presenting cells, such as dendritic cells, and the appropriate immunogenic peptide. After an appropriate incubation time (typically about 7-28 days), in which the precursor cells are activated and expanded into effector cells, the cells are infused back into the patient, where they will destroy (CTL) or facilitate destruction (HTL) of their specific target cell (an infected cell or a tumor cell). Transfected dendritic cells may also be used as antigen presenting cells.

The vaccine compositions of the invention can also be used in combination with other treatments used for cancer, including use in combination with immune adjuvants such as IL-2, IL-12, GM-CSF, and the like.

The vaccine compositions of the invention may also be used therapeutically in combination with treatments such as surgery. An example is a situation in which a patient has undergone surgery to remove a primary tumor and the vaccine is then used to slow or prevent recurrence and/or metastasis.

Where susceptible individuals, e.g., individuals who may be diagnosed as being genetically pre-disposed to developing a prostate tumor, are identified prior to diagnosis of cancer, the composition can be targeted to them, thus minimizing the need for administration to a larger population.

The dosage for an initial therapeutic immunization generally occurs in a unit dosage range where the lower value is about 1, 5, 50, 500, or 1,000 μg and the higher value is about 10,000; 20,000; 30,000; or 50,000 μg. Dosage values for a human typically range from about 500 μg to about 50,000 μg per 70 kilogram patient. Initial doses followed by boosting doses at established intervals, e.g., from four weeks to six months, may be required, possibly for a prolonged period of time to effectively treat a patient. Boosting dosages of between about 1.0 μg to about 50,000 μg of peptide pursuant to a boosting regimen over weeks to months may be administered depending upon the patient's response and condition as determined by measuring the specific activity of CTL and HTL obtained from the patient's blood.

When DNA is used to induce an immune response, it is administered as naked or formulated DNA, generally in a dose range of approximately 1-5 mg (e.g., 0.5 mg, 1 mg, 2 mg, 3 mg, 4 mg), or via the ballistic “gene gun” delivery, typically in a dose range of approximately 10-100 μg. The DNA can be delivered in a variety of conformations, e.g., linear, circular etc. Various viral vectors have also successfully been used that comprise nucleic acids which encode epitopes in accordance with the invention using generally the same dose range as naked DNA (e.g. approximately 1-5 mg).

Administration should continue until at least clinical symptoms or laboratory tests indicate that the tumor has been eliminated or that the tumor cell burden has been substantially reduced and for a period thereafter. The dosages, routes of administration, and dose schedules are adjusted in accordance with methodologies known in the art.

In certain embodiments, polypeptides, analog polynucleotides, and compositions of the present invention are employed in serious disease states, that is, life-threatening or potentially life threatening situations. In such cases, as a result of the minimal amounts of extraneous substances and the relative nontoxic nature of the polypeptides in preferred compositions of the invention, it is possible and may be felt desirable by the treating physician to administer substantial excesses of these polypeptide compositions relative to these stated dosage amounts.

The vaccine compositions of the invention can also be used as prophylactic agents. For example, the compositions can be administered to individuals at risk of developing prostate cancer. Generally, the dosage for an initial prophylactic immunization occurs in a unit dosage range where the lower value is about 1, 5, 50, 500, or 1000 μg and the higher value is about 10,000; 20,000; 30,000; or 50,000 μg. Dosage values for a human typically range from about 500 μg to about 50,000 μg per 70 kilogram patient. This is followed by boosting dosages of between about 1.0 μg to about 50,000 μg of peptide administered at defined intervals from about four weeks to six months after the initial administration of vaccine. The immunogenicity of the vaccine may be assessed by measuring the specific activity of CTL and HTL obtained from a sample of the patient's blood.

The pharmaceutical compositions for therapeutic treatment are intended for parenteral, topical, oral, intrathecal, or local administration. Preferably, the pharmaceutical compositions are administered parentally, e.g., intravenously, subcutaneously, intradermally, or intramuscularly. Thus, the invention provides compositions for parenteral administration which comprise a solution of the immunogenic polypeptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH-adjusting and buffering agents, tonicity adjusting agents, wetting agents, preservatives, and the like, for example, sodium acetate, sodium lactate, sodium chloride, potassium chloride, calcium chloride, sorbitan monolaurate, triethanolamine oleate, etc.

The concentration of polypeptides or analogs of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

A human unit dose form of the polypeptide B or polynucleotide composition is typically included in a pharmaceutical composition that comprises a human unit dose of an acceptable carrier, preferably an aqueous carrier, and is administered in a volume of fluid that is known by those of skill in the art to be used for administration of such compositions to humans (see, e.g., Remington's Pharmaceutical Sciences, 17^{th} Edition, A. Gennaro, Editor, Mack Publishing Co., Easton, Pa., 1985).

7. Use of Analog Epitopes as Diagnostic Agents and for Evaluating Immune Responses

In one embodiment of the invention, heteroclitic analog polypeptides as described herein are used as reagents to evaluate an immune response. The immune response to be evaluated is induced by using as an immunogen any agent that may result in the induction of antigen-specific CTLs that recognize and bind to the analog polypeptide to be employed as the reagent. The polypeptide need not be used as the immunogen. Assay systems that are used for such an analysis include relatively recent technical developments such as tetramers, staining for intracellular lymphokines and interferon release assays, or ELISPOT assays.

For example, polypeptides of the invention are used in tetramer staining assays to assess peripheral blood mononuclear cells for the presence of antigen-specific CTLs following exposure to a tumor cell antigen or an immunogen. The HLA-tetrameric complex is used to directly visualize antigen-specific CTLs (see, e.g., Ogg et al., Science 279:2103-2106, 1998; and Altman et al., Science 174:94-96, 1996) and determine the frequency of the antigen-specific CTL population in a sample of peripheral blood mononuclear cells. A tetramer reagent using a peptide of the invention is generated as follows: A peptide that binds to an HLA molecule is refolded in the presence of the corresponding HLA heavy chain and β₂-microglobulin to generate a trimolecular complex. The complex is biotinylated at the carboxyl terminal end of the heavy chain at a site that was previously engineered into the protein. Tetramer formation is then induced by the addition of streptavidin. By means of fluorescently labeled streptavidin, the tetramer can be used to stain antigen-specific cells. The cells can then be identified, for example, by flow cytometry. Such an analysis may be used for diagnostic or prognostic purposes. Cells identified by the procedure can also be used for therapeutic purposes.

Polypeptides of the invention are also used as reagents to evaluate immune recall responses (see, e.g., Bertoni, et al., J. Clin. Invest. 100:503-513, 1997 and Penna, et al., J. Exp. Med. 174:1565-1570, 1991). For example, PBMC samples from individuals with cancer are analyzed for the presence of antigen-specific CTLs using specific peptides. A blood sample containing mononuclear cells can be evaluated by cultivating the PBMCs and stimulating the cells with a peptide of the invention. After an appropriate cultivation period, the expanded cell population can be analyzed, for example, for CTL activity.

The polypeptides are also used as reagents to evaluate the efficacy of a vaccine. PBMCs obtained from a patient vaccinated with an immunogen are analyzed using, for example, either of the methods described above. The patient is HLA typed, and analog polypeptide that recognizes the allele-specific molecules present in that patient are used for the analysis. The immunogenicity of the vaccine is indicated by the presence of epitope-specific CTLs in the PBMC sample.

The polypeptides of the invention are also used to make antibodies, using techniques well known in the art (see, e.g. CURRENT PROTOCOLS IN IMMUNOLOGY, Wiley/Greene, NY; and Antibodies A Laboratory Manual, Harlow and Lane, Cold Spring Harbor Laboratory Press, 1989), which may be useful as reagents to diagnose or monitor cancer. Such antibodies include those that recognize a peptide in the context of an HLA molecule, i.e., antibodies that bind to a peptide-MHC complex.

8. Kits

The polypeptide and polynucleotide compositions of this invention can be provided in kit form together with instructions for vaccine administration. Typically the kit would include desired polypeptide compositions in a container, preferably in unit dosage form and instructions for administration. An alternative kit would include a polynucleotide (e.g., minigene) construct with desired nucleic acids of the invention in a container, preferably in unit dosage form together with instructions for administration. Lymphokines such as IL-2 or IL-12 may also be included in the kit. Other kit components that may also be desirable include, for example, a sterile syringe, booster dosages, etc.

Heteroclitic analogs have successfully been used to induce an immune response. Immune responses with such analogs have been induced by administering the analogs in various forms. Upon administration of peptide-based analog forms, immune responses have been induced by direct loading of an analog onto an empty HLA molecule that is expressed on a cell, and via internalization of the analog and processing via the HLA class I pathway; in either event, the HLA molecule expressing the analog was then able to interact with and induce a CTL response.

Accordingly compositions in accordance with the invention exist in several forms as described throughout this disclosure. Embodiments of each of these composition forms in accordance with the invention can successfully induce an immune response. A kit may comprise any of these compositions.

9. Class I Motifs

In the past few years, evidence has accumulated to demonstrate that a large fraction of HLA class I molecules can be classified into a relatively few supertypes, each characterized by largely overlapping peptide binding repertoires, and consensus structures of the main peptide binding pockets. Thus, peptides of the present invention are identified by any one of several HLA-specific amino acid motifs (see, e.g., Tables 3-4), or if the presence of the motif corresponds to the ability to bind several allele-specific HLA antigens, a supermotif. The HLA molecules that bind to peptides that possess a particular amino acid supermotif are collectively referred to as an HLA “supertype.”

For the convenience of the reader, the peptide motifs and supermotifs described below, and summarized in Tables 3-4, provide guidance for the identification and use of analog polypeptides in accordance with the invention. This will permit identification of candidate wild-type epitopes corresponding to various class I motifs different from those illustrated in the examples below or epitopes bearing those motifs illustrated below but in different antigens in order to apply the rules set forth herein to construct analogs.

Heteroclitic analogs can be designed according to the methods of the invention from a peptide, without regard to the motif or supermotif to which the peptide belongs. The primary anchor residues of the HLA class I peptide epitope supermotifs and motifs delineated below are summarized in Table 3. The HLA class I motifs set out in Table 4 are those most particularly relevant to the invention claimed here. Allele-specific HLA molecules that comprise HLA class I supertype families are listed in Table 5. In some cases, peptide epitopes may be listed in both a motif and a supermotif. The relationship of a particular motif and respective supermotif is indicated in the description of the individual motifs.
i. HLA-A1 Supermotif

The HLA-A1 supermotif is characterized by the presence in peptide ligands of a small (T or S) or hydrophobic (L, I, V, or M) primary anchor residue in position 2, and an aromatic (Y, F, or W) primary anchor residue at the C-terminal position of the epitope. The corresponding family of HLA molecules that bind to the A1 supermotif (i.e., the HLA-A1 supertype) is comprised of at least A*0101, A*2601, A*2602, A*2501, and A*3201 (see, e.g., DiBrino, M. et al., J. Immunol. 151:5930, 1993; DiBrino, M. et al., J. Immunol. 152:620, 1994; Kondo, A. et al., Immunogenetics 45:249, 1997). Other allele-specific HLA molecules predicted to be members of the A1 superfamily are shown in Table 5.
ii. HLA-A2 Supermotif

Primary anchor specificities for allele-specific HLA-A2.1 molecules (see, e.g., Falk et al., Nature 351:290-296, 1991; Hunt et al., Science 255:1261-1263, 1992; Parker et al., J. Immunol. 149:3580-3587, 1992; Ruppert et al., Cell 74:929-937, 1993) and cross-reactive binding among HLA-A2 and -A28 molecules have been described. (See, e.g., Fruci et al., Human Immunol. 38:187-192, 1993; Tanigaki et al., Human Immunol. 39:155-162, 1994; Del Guercio et al., J. Immunol. 154:685-693, 1995; Kast et al., J. Immunol. 152:3904-3912, 1994 for reviews of relevant data.) These primary anchor residues define the HLA-A2 supermotif; which presence in peptide ligands corresponds to the ability to bind several different HLA-A2 and -A28 molecules. The HLA-A2 supermotif comprises peptide ligands with L, I, V, M, A, T, or Q as a primary anchor residue at position 2 and L, I, V, M, A, or T as a primary anchor residue at the C-terminal position of the epitope.

The corresponding family of HLA molecules (i.e., the HLA-A2 supertype that binds these peptides) is comprised of at least: A*0201, A*0202, A*0203, A*0204, A*0205, A*0206, A*0207, A*0209, A*0214, A*6802, and A*6901. Other allele-specific HLA molecules predicted to be members of the A2 superfamily are shown in Table 5.
iii. HLA-A3 Supermotif

The HLA-A3 supermotif is characterized by the presence in peptide ligands of A, L, I, V, M, S, or, T as a primary anchor at position 2, and a positively charged residue, R or K, at the C-terminal position of the epitope, e.g., in position 9 of 9-mers (see, e.g., Sidney et al., Hum. Immunol. 45:79, 1996). Exemplary members of the corresponding family of HLA molecules (the HLA-A3 supertype) that bind the A3 supermotif include at least A*0301, A*1101, A*3101, A*3301, and A*6801. Other allele-specific HLA molecules predicted to be members of the A3 supertype are shown in Table 5.
iv. HLA-A24 Supermotif

The HLA-A24 supermotif is characterized by the presence in peptide ligands of an aromatic (F, W, or Y) or hydrophobic aliphatic (L, L, V, M, or T) residue as a primary anchor in position 2, and Y, F, W, L, I, or M as primary anchor at the C-terminal position of the epitope (see, e.g., Sette and Sidney, Immunogenetics, 50:201-212,1999). The corresponding family of HLA molecules that bind to the A24 supermotif (i.e., the A24 supertype) includes at least A*2402, A*3001, and A*2301. Other allele-specific HLA molecules predicted to be members of the A24 supertype are shown in Table 5.
v. HLA-B7 Supermotif

The HLA-B7 supermotif is characterized by peptides bearing proline in position 2 as a primary anchor, and a hydrophobic or aliphatic amino acid (L, I, V, M, A, F, W, or Y) as the primary anchor at the C-terminal position of the epitope. The corresponding family of HLA molecules that bind the B7 supermotif (i.e., the HLA-B7 supertype) is comprised of at least twenty six HLA-B proteins including: B*0702, B*0703, B*0704, B*0705, B*1508, B*3501, B*3502, B*3503, B*3504, B*3505, B*3506, B*3507, B*3508, B*5101, B*5102, B*5103, B*5104, B*5105, B*5301, B*5401, B*5501, B*5502, B*5601, B*5602, B*6701, and B*7801 (see, e.g., Sidney, et al., J. Immunol. 154:247, 1995; Barber, et al., Curr. Biol. 5:179, 1995; Hill, et al., Nature 360:434, 1992; Rammensee, et al., Immunogenetics 41:178, 1995 for reviews of relevant data). Other allele-specific HLA molecules predicted to be members of the B7 supertype are shown in Table 5.
vi. HLA-B27 Supermotif

The HLA-B27 supermotif is characterized by the presence in peptide ligands of a positively charged (R, H, or K) residue as a primary anchor at position 2, and a hydrophobic (F, Y, L, W, M, L A, or V) residue as a primary anchor at the C-terminal position of the epitope (see, e.g. Sette and Sidney, Immunogenetics, 50:201-212, 1999). Exemplary members of the corresponding family of HLA molecules that bind to the B27 supermotif (i.e., the B27 supertype) include at least B*1401, B*1402, B*1509, B*2702, B*2703, B*2704, B*2705, B*2706, B*3801, B*3901, B*3902, and B*7301. Other allele-specific HLA molecules predicted to be members of the B27 supertype are shown in Table 5.
vii. HLA-B44 Supermotif

The HLA-B44 supermotif is characterized by the presence in peptide ligands of negatively charged (D or E) residues as a primary anchor in position 2, and hydrophobic residues (F, W, Y, L, I, M, V, or A) as a primary anchor at the C-terminal position of the epitope (see, e.g., Sidney et al., Immunol. Today 17:261, 1996). Exemplary members of the corresponding family of HLA molecules that bind to the B44 supermotif (i.e., the B44 supertype) include at least: B*1801, B*1802, B*3701, B*4001, B*4002, B*4006, B*4402, B*4403, and B*4006.
viii. HLA-B58 Supermotif

The HLA-B58 supermotif is characterized by the presence in peptide ligands of a small aliphatic residue (A, S, or T) as a primary anchor residue at position 2, and an aromatic or hydrophobic residue (F, W, Y, L, I, V, M, or A) as a primary anchor residue at the C-terminal position of the epitope (see, e.g., Sette and Sidney, Immunogenetics, 50:201-212, 1999 for reviews of relevant data). Exemplary members of the corresponding family of HLA molecules that bind to the B58 supermotif (i.e., the B58 supertype) include at least: B*1516, B*1517, B*5701, B*5702, and B*5801. Other allele-specific HLA molecules predicted to be members of the B58 supertype are shown in Table 5.
ix. HLA-B62 Supermotif

The HLA-B62 supermotif is characterized by the presence in peptide ligands of the polar aliphatic residue Q or a hydrophobic aliphatic residue (L, V, M, L or P) as a primary anchor in position 2, and a hydrophobic residue. (F, W, Y, M, L V, L, or A) as a primary anchor at the C-terminal position of the epitope (see, e.g., Sette and Sidney, Immunogenetics, 50:201-212, 1999). Exemplary members of the corresponding family of HLA molecules that bind to the B62 supermotif (i.e., the B62 supertype) include at least: B*1501, B*1502, B*1513, and B5201. Other allele-specific HLA molecules predicted to be members of the B62 supertype are shown in Table 5.
x. HLA-A1 Motif

The HLA-A1 motif is characterized by the presence in peptide ligands of T, S, or M as a primary anchor residue at position 2 and the presence of Y as a primary anchor residue at the C-terminal position of the epitope. An alternative allele-specific A1 motif is characterized by a primary anchor residue at position 3 rather than position 2. This motif is characterized by the presence of D, E, A, or S as a primary anchor residue in position 3, and a Y as a primary anchor residue at the C-terminal position of the epitope (see, e.g., DiBrino et al., J. Immunol., 152:620, 1994; Kondo et al., Immunogenetics 45:249, 1997; and Kubo et al., J. Immunol. 152:3913, 1994 for reviews of relevant data).
xi. HLA-A*0201 Motif

An HLA-A2*0201 motif was determined to be characterized by the presence in peptide ligands of L or M as a primary anchor residue in position 2, and L or V as a primary anchor residue at the C-terminal position of a 9-residue peptide (see, e.g., Falk et al., Nature 351:290-296, 1991) and was further found to comprise an I at position 2 and I or A at the C-terminal position of a nine amino acid peptide (see, e.g., Hunt et al., Science 255:1261-1263, Mar. 6, 1992; Parker et al., J. Immunol. 149:3580-3587, 1992). The A*0201 allele-specific motif has also been defined by the present inventors to additionally comprise V, A, T, or Q as a primary anchor residue at position 2, and M or T as a primary anchor residue at the C-terminal position of the epitope (see, e.g., Kast et al., J. Immunol. 152:3904-3912, 1994). Thus, the HLA-A*0201 motif comprises peptide ligands with L, I, V, M, A, T, or Q as primary anchor residues at position 2 and L, I, V, M, A, or T as a primary anchor residue at the C-terminal position of the epitope. The preferred and tolerated residues that characterize the primary anchor positions of the HLA-A*0201 motif are identical to the residues describing the A2 supermotif.
xii. HLA-A3 Motif

The HLA-A3 motif is characterized by the presence in peptide ligands of L, M, V, I, S, A, T, F, C, G, or D as a primary anchor residue at position 2, and the presence of K, Y, R, H, F, or A as a primary anchor residue at the C-terminal position of the epitope (see, e.g., DiBrino et al., Proc. Natl. Acad. Sci USA 90:1508, 1993; and Kubo et al., J. Immunol. 152:3913-3924, 1994).
xiii. HLA-A11 Motif

The HLA-A11 motif is characterized by the presence in peptide ligands of V, T, M, L, I, S, A, G, N, C, D, or F as a primary anchor residue in position 2, and K, R, Y, or H as a primary anchor residue at the C-terminal position of the epitope (see, e.g., Zhang et al., Proc. Natl. Acad. Sci USA 90:2217-2221, 1993; and Kubo et al., J. Immunol. 152:3913-3924, 1994).
xiv. HLA-A24 Motif

The HLA-A24 motif is characterized by the presence in peptide ligands of Y, F, W, or M as a primary anchor residue in position 2, and F, L, I, or W as a primary anchor residue at the C-terminal position of the epitope (see, e.g., Kondo et al., J. Immunol. 155:4307-4312, 1995; and Kubo et al., J. Immunol. 152:3913-3924, 1994).

10. Assays to Detect T-Cell Responses

Once heteroclitic analogs of the invention are synthesized, they can be tested for the ability to elicit a T-cell response. The preparation and evaluation of motif-bearing peptides such as heteroclitic analogs are described in PCT publications WO 94/20127 and WO 94/03205. Briefly, peptides comprising epitopes from a particular antigen are synthesized and tested for their ability to bind to the appropriate HLA proteins. These assays may involve evaluating the binding of a peptide of the invention to purified HLA class I molecules in relation to the binding of a radioiodinated reference peptide. Alternatively, cells expressing empty class I molecules (i.e. lacking peptide therein) may be evaluated for peptide binding by immunofluorescent staining and flow microfluorimetry. Other assays that may be used to evaluate peptide binding include peptide-dependent class I assembly assays and/or the inhibition of CTL recognition by peptide competition. Those peptides that bind to the class I molecule, typically with an affinity of 500 nM or less, are further evaluated for their ability to serve as targets for CTLs derived from infected or immunized individuals, as well as for their capacity to induce primary in vitro or in vivo CTL responses that can give rise to CTL populations capable of reacting with selected target cells associated with a disease.

Conventional assays utilized to detect T cell responses include proliferation assays, lymphokine secretion assays, direct cytotoxicity assays, and limiting dilution assays. Such assays are useful in comparing the induction of immune responses by heteroclitic analog peptides to response induced by non-heteroclitic analogs class I peptides (e.g., from which the heteroclitic analog sequence was based). For example, antigen-presenting cells that have been incubated with a peptide can be assayed for the ability to induce CTL responses in responder cell populations. Antigen-presenting cells can be normal cells such as peripheral blood mononuclear cells or dendritic cells. Alternatively, mutant non-human mammalian cell lines that are deficient in their ability to load class I molecules with internally processed peptides and that have been transfected with the appropriate human class I gene, may be used to test for the capacity of the peptide to induce in vitro primary CTL responses.

Peripheral blood mononuclear cells (PBMCs) may be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived.

Additionally, a method has been devised which allows direct quantification of antigen-specific T cells by staining with fluorescein-labelled HLA tetrameric complexes (Altman, J. D. et al., Proc. Natl. Acad. Sci. USA 90:10330, 1993; Altman, J. D. et al., Science 274:94, 1996). Other relatively recent technical developments include staining for intracellular lymphokines, and interferon-γ release assays or ELISPOT assays. Tetramer staining, intracellular lymphokine staining and ELISPOT assays all appear to be at least 10-fold more sensitive than more conventional assays (Lalvani, A. et al., J. Exp. Med. 186:859, 1997; Dunbar, P. R. et al., Curr. Biol. 8:413, 1998; Murali-Krishna, K. et al., Immunity 8:177, 1998).

If desired, HTL activation may also be assessed using such techniques known to those in the art such as T cell proliferation and secretion of lymphokines, e.g. IL-2 (see, e.g. Alexander, J. et al., Immunity 1:751-761, 1994).

Alternatively, immunization of HLA transgenic mice can be used to determine immunogenicity of peptide epitopes. Several transgenic mouse models including mice with human A2.1, A11 (which can additionally be used to analyze HLA-A3 epitopes), and B7 alleles have been characterized and others (e.g., transgenic mice for HLA-A1 and A24) are being developed. HLA-DR1 and HLA-DR3 mouse models have also been developed. Additional transgenic mouse models with other HLA alleles may be generated as necessary. The mice may be immunized with peptides emulsified in Incomplete Freund's Adjuvant and the resulting T cells tested for their capacity to recognize peptide-pulsed target cells and target cells transfected with appropriate genes. CTL responses may be analyzed using cytotoxicity assays described above. Similarly, HTL responses may be analyzed using such assays as T cell proliferation or secretion of lymphokines.

Heteroclitic analogs of the invention often induce both Th1 and Th2 cytokine responses. Therefore, one method to compare a heteroclitic candidate with a preselected class I peptide is to test the induction of Th1 and Th2 cytokines. The preselected class I peptide will typically be a peptide from which the heteroclitic analog is derived, or if such a peptide does not exist, a class I peptide with the highest similarity to the candidate. Heteroclitic analogs of the invention typically induce both Th1 and Th2 cytokine responses, but at a level greatly enhanced compared to the class I peptide from which the analog was derived. For example, a given heteroclitic analog will stimulate an equivalent level of Th1 or Th2 cytokine (50 to 100 pg/ml) at a 10-fold or lower dose compared to the wild-type peptide from which the analog was derived. Additionally, where the class I peptide induces only, or mainly, either a Th1 or Th2 response, a heteroclitic analog may induce both Th1 and Th2 responses. Th1 cytokines include, e.g., IFN-γ, IL-2 and IL-3. Th2 cytokines include, e.g., IL-4, IL-5, IL-6 and IL-10. Production of cytokines can be measured, for example, using ELISA or other immunological quantitation methods. See, e.g., McKinney, et al. Journal of Immunological Methods 237:105-117 (2000).

### EXAMPLES

### Preparation A

### Peptide Synthesis and Generation of Peptide Analogs

The peptides used in these examples are shown in Table 1. All of the wild-type human CTL epitopes derived from tumor-associated antigens have shown immunogenicity in human and transgenic mouse systems (Kawashima, I., et al., Human Immunol. (1998) 59:1; Ishioka, G., et al., J. Immunol. (1999) 162:3915; Epimmune, unpublished data).

Peptides that were tested initially for heteroclitic activity were synthesized by Chiron Technologies (Victor, Australia). Peptides requiring further biological characterization were synthesized at Epimmune using conventional methods (Ruppert, J., et al., Cell (1993) 74:929) and their purity was routinely >95%, as determined by analytical reverse-phase HPLC. The identity of the latter peptides was confirmed by mass spectral analysis.

### Preparation B

### Scheme for Selection of Single Amino Acid Substitutions

Table 2 shows the similarity assignments between any given amino acid pair so that a given amino acid substitution could be characterized as being a conservative, semi-conservative, or non-conservative substitution.

The degree of similarity between amino acid pairs was quantified by averaging, for each amino acid pair, the rank coefficient scores for PAM250, hydrophobicity, and side chain volume as described below. Based on the average values of these composite rankings, the table shows each pair to be conserved, semi-conserved or non-conserved.

The Dayhoff PAM250 score (Dayhoff, M. O., et al., Atlas of Protein Sequence and Structure, Vol. 5, suppl.3. (1978) M. O. Dayhoff, ed. National Biomedical Research Foundation, Washington DC, p. 345; Creighton, T. E., Proteins: structures and molecular properties (1993) (2nd edition) W. H. Freeman and Company, NY; http://prowl.rockefeller.edu/aainfo/pam250. html) is a commonly utilized protein alignment scoring matrix which measures the percentage of acceptable point mutations (PAM) within a defined time frame. The frequencies of these mutations are different from what would be expected from the probability of random mutations, and presumably reflect a bias due to the degree of physical and chemical similarity of the amino acid pair involved in the substitution. To obtain a score of amino acid similarity that could be standardized with other measures of similarity, the PAM250 scores were converted to a rank value, where 1 indicates the highest probability of being an accepted mutation.

The most commonly utilized scales to represent the relative hydrophobicity of the 20 naturally occurring amino acids (Cornette, J., et al., J. Mol. Biol. (1987) 195:659) are those developed on the basis of experimental data by Kyte and Doolittle (Kyte, J. and R. F. Doolittle, J. Mol. Biol. (1982) 157:105), and by Fauchere and Pliska (Fauchere, J. and V. Pliska, Eur. J. Med. Chem. (1983) 18:369). The Kyte/Doolittle scale measures the H₂O/organic solvent partition of individual amino acids. Because it considers the position of amino acids in folded proteins, it may most accurately reflect native hydrophobicity in the context of proteins. The Fauchere/Pliska scale measures the octanol/H₂O partitioning of N-acetyl amino acid amides, and most accurately reflects hydrophobicity in the context of denatured proteins and/or small synthetic peptides. To obtain scores for hydrophobicity, each amino acid residue was ranked on both the Kyte/Doolittle and Fauchere/Pliska hydrophobicity scales. An average rank between the two scales was calculated and the average difference in hydrophobicity for each pair was calculated.

Finally, for calculating amino acid side-chain volume, the partial volume in solution obtained by noting the increase in volume of water after adding either one molecule or one gram of amino acid residue was considered (Zamyatnin, A. A., Ann. Rev. Biophys. Bioeng. (1984) 13:145; Zamyatnin, A. A., Prog. Biophys. Mol. Biol. (1972) 24:107). The absolute difference in the partial volume of each possible pairing of the 20 naturally occurring amino acids was calculated and ranked, where 1 indicated residues with the most similar volumes, and 20 the most dissimilar.

### Preparation C

### Materials for Assays

1. APC Lines

Cell lines that present peptides in the context of HLA-A2.1 were prepared as follows:

The 0.221A2.1 cell line was generated by transfecting the HLA-A2.1 gene into the HLA-A, -B, -C-null mutant EBV-transformed human B-lymphoblastoid cell line 3A4-721.221 (Kawashima, I., et al., Human Immunol. (1998) 59:1). The cell line GM3107 was used as APCs to measure B7 CTL responses.

Tumor cell lines were prepared by transfection of Meth A cells, a methylcholanthrene-induced sarcoma, and the Jurkat cell line with the HLA-A2.1 or HLA-A2.1/K^{b} transgene transfection was performed using methods described elsewhere (Vitiello, A., et al., J. Exp. Med. (1991) 173:1007). A combination of the HLA-typed melanoma cell lines 624mel (A2.1⁺, MAGE⁺) and 888mel (A2.1⁻, MAGE⁻), were kindly provided by Y. Kawakami and S. Rosenberg (National Cancer Institute), and were used to measure presentation of endogenously processed MAGE3 epitopes (Boon, T., et al., Ann. Rev. Immunol. (1994) 12:337). The melanoma cell lines were treated with 100 IU/ml human IFN-γ (Genzyme, Cambridge, Mass.) for 48 h at 37° C. before using as APC.

All cells in this study were grown in RPMI-1640 medium supplemented with antibiotics, sodium pyruvate, nonessential amino acids, and 10% (v/v) heat-inactivated FBS.

2. In Vitro Induction of CTL from Human PBMC and Derivation of Human CTL Lines

To generate peptide-specific CTL lines against the MAGE3.112, MAGE2.170, and a carcinoembryonic antigen (CEA) epitope, CEA.691, PBMC from normal subjects were stimulated repeatedly in vitro with peptide as described (Kawashima, I., et al., Human Immunol. (1998) 59:1). Briefly, peptide-pulsed dendritic cells (differentiated from adherent PBMC by culturing in GM-CSF and IL4) were co-cultured with autologous CD8⁺ T cells, obtained by positive selection with antibody-coated beads (Dynal A. S., Oslo, Norway or Miltenyi Biotec, Auburn, Calif.) in a 48-well plate. After 7 days of culture in the presence of IL2, IL7, and IL10, each PBMC culture (well) was restimulated in vitro with adherent PBMC pulsed with peptide. Cultures were then tested for CTL activity by measuring IFN-γ production after stimulation with 0.221A2.1 tumor APC (A2 epitopes) or GM3107 tumor cells (137 epitopes), in the presence or absence of peptide. CTL lines were expanded from PBMC cultures demonstrating peptide-specific IFN-γ responses by additional in vitro stimulation with adherent peptide-pulsed PBMC.

3. Murine CTL Lines

CTL lines against epitopes HBV Pol.455 and HIV Pol.476 peptides were generated in HLA-A2.1/K^{bxs} transgenic mice by DNA immunization as described elsewhere (Ishioka, G., et al., J. Immunol. (1999) 162:3915). HLA-A2.1/K^{bxs} and HLA-A2.1/K^{bxd} transgenic mice were bred at Epimmune. These strains represent the F1 generation of a cross between an HLA-A2.1/K^{b} transgenic strain generated on the C57BL/6 background (Vitiello, A., et al., J. Exp. Med. (1991) 173:1007), and SJL or BALB/c mice (Jackson Laboratories, Bar Harbor, Me.), respectively. CTL lines against the MAGE2.157 epitope were generated by immunizing 8-12 wk old HLA-A2.1/K^{bxs} mice s.c. at the tail base with 50 μg of peptide and 140 μg of the HBV Core.128 Th epitope, TPPAYRPPNAPIL (SEQ ID NO:34), emulsified in IFA and restimulating primed splenocytes repeatedly in vitro with peptide.

### Preparation D

### Assay Methods

1. Measurement of Peptide Binding Affinity for HLA-A2.1 or HLA-B7 Molecules

Binding of test peptides to HLA-A2.1 was measured by determining the level of competition induced by a given test peptide for binding of a radiolabeled standard peptide to HLA-A2.1. The percentage of MHC-bound radioactivity was determined by gel filtration and the concentration of test peptide that inhibited 50% of the binding of the labeled standard peptide (IC₅₀) was calculated (Ruppert, J., et al., Cell (1993) 74:929; Sette, A., et al., Mol. Immunol. (1994) 31:813). The standard peptide was the HBV Core.18 epitope (sequence FLPSDFFPSV) (SEQ ID NO:35). A similar assay was performed to determine the binding affinity of peptides to purified HLA-B7 (B*0702) molecules. In the latter assay, the radiolabeled standard peptide was the SS 5-13a (L₇→Y) peptide (sequence APRTLVYLL) (SEQ ID NO:36).

2. Measurement of Murine and Human IFN-γ, IL-5, and IL-10 Production by CTL

An in situ capture ELISA was used for measuring IFN-γ release from CTL (McKinney, D., et al., J. Immunol. Methods (2000) 237:105). Briefly, CTL were stimulated with APC and peptide in ELISA-grade 96-well flat bottom wells that were precoated with either an anti-mouse IFN-γ (clone R4-6A2, Pharmingen, San Diego, Calif.) or anti-human IFN-γ mAb (clone NIB42, Pharmingen). After culturing cells, wells are washed and developed by adding a biotinylated anti-mouse IFN-γ (clone XMG1.2, Pharmingen) or anti-human IFN-γ (clone 4S.B3, Pharmingen) mAb followed by enzyme-conjugated streptavidin (Zymed, South San Francisco, Calif.) and 3,3′,5,5′ tetramethylbenzidine substrate (ImmunoPure TMB substrate kit, Pierce, Rockford, Ill.). The absorbance of each well was measured at 450 nm on a Labsystems Multiskan RC ELISA plate reader. The level of IFN-γ produced in each well was determined by extrapolation from a mouse or human IFN-γ standard curve established in the same assay.

Murine and human IL-5 and IL-10 were measured in culture supernates using ELISA kits (R&D Biosystems, Minneapolis, Minn.). These assays, employing the quantitative sandwich ELISA technique, were performed according to the manufacturer's protocol.

3. Enzyme-Liked Immunospot (ELISPOT) Assay for Measuring Ex Vivo CTL Responses

ELISPOT assays were performed according to standard protocols (Murali-Krishna, K., et al., Immunity (1998) 8:177; Lewis, J. J., et al., Int. J. Cancer (2000) 87:391). Briefly, flat bottom 96-well nitrocellulose plates (Immobilon-P membrane, Millipore, Bedford, Mass.) were coated with anti-IFN-γ mAb (10 μg/ml, clone R4-6A2) and incubated overnight at 4° C. After washing with PBS, plates were blocked with RPMI medium containing 10% FBS for 1 h at 37° C. Four×10⁵ splenic CD8⁺ cells isolated by magnetic beads (Miltenyi, Auburn, Calif.) and 5×10⁴ Jurkat-A2.1/K^{b} cells pulsed with 10 μg/ml of peptide were added to each well and cells were incubated for 20 h in RPMI medium containing 10% FBS. After incubation, the plates were washed thoroughly with PBS/0.05% Tween and biotinylated anti-IFN-γ mAb (2 μg/ml, clone XMG1.2) was added to each well and plates were incubated for 4 h at 37° C. Plates were then washed four times with PBS (containing 0.1% Tween-20) and Vectastain ABC peroxidase (Vectastain Elite kit; Vector Laboratories, Burlingame, Calif.). After incubating for 1 h at room temperature, plates were washed three times with 1×PBS/0.05% Tween followed by three additional washes with 1×PBS. One hundred μl of AEC solution (Sigma Chemical, St. Louis, Mo.) was added to develop the spots. The reaction was stopped after 4-6 min under running tap water. The spots were counted by computer-assisted image analysis (Zeiss KS ELISPOT Reader, Jena, Germany). The net number of spots/10⁶ CD8⁺ cells was calculated as follows: [(number of spots against relevant peptide)−(number of spots against irrelevant control peptide)]33 2.5.

### Example 1

### Screening of Peptide Analogs for Heteroclitic Activity

To determine rules for designing heteroclitics, several different CTL lines were screened for reactivity against panels of analogs. Modification of T cell stimulatory capacity was achieved with no alternation of the primary MHC anchors.

The wild-type epitopes include tumor epitopes derived from self-antigens that are specifically up-regulated in epithelial cell cancers and have been shown to be immunogenic. Viral epitopes used, such as those from the polymerase genes of the HIV and HBV, have been shown to be immunogenic as well.

The rules described herein provide a basis to design heteroclitic analogs, drastically reducing the screening otherwise required and are extremely useful in designing epitope-based vaccines for cancer and infectious diseases.

In some of the examples set forth below, 17% of the total analogs screened (which fit the heteroclicity rules disclosed herein) were heteroclitic (16/95). This is significant for two reasons: first, the efficiency of detecting heteroclitics increased from 2.2% to 17% by employing analogs that follow the rules of heteroclitic substitution; second, the number of peptides which need to be synthesized is reduced dramatically from about a 100 analogs per epitope to about 15 analogs per epitope, making the process cost effective and amenable to high throughput. Through the application of the heteroclitic substitution rules of the invention, the efficiency of generating heteroclitic analogs was increased nearly 100 to 1000-fold, from 0.2% (4 identified from screening of 233 CEA.691 and MAGE3.112 analogs) to 33% (3 identified by screening of 9 predicted analogs). The latter frequency may be a gross underestimate since only 4 of 6 analogs showing potential heteroclitic activity in initial assays were subjected to further analysis.

To summarize, in a set of experiments, Applicants have identified heteroclitic analogs of a number of different HLA-A2.1 and B7-restricted CTL epitopes of a cancerous origin. The relevant wild-type epitopes and analogs are shown in Table 1. All these epitopes have been shown to be immunogenic in our earlier reports (Kawashima, et al., Human Immunology 59:1-14 (1998), Ishioka, et al., J. Immunol. 162(7):3915-25 (1999)). In initial experiments, the antigenicity of 233 analogs of the CEA.691 and MAGE3.112 CTL epitopes was investigated. The nature of the four heteroclitic analogs identified suggested that heteroclitic substitutions involved conservative substitutions at positions 3, 5 and 7. This hypothesis was tested in a subsequent study involving three additional epitopes MAGE2.157, HIVPol.476, and HBVPol.455. All of the heteroclitic analogs thus identified conformed to the rules proposed, namely that heteroclitic analogs were associated with conservative or semi-conservative substitutions at positions 3, 5 and/or 7.

To more closely mimic the clinical application of heteroclitic analogs in cancer immunotherapy, the murine epitope, p53.261 was also modified. A partial state of T cell tolerance has been reported for this epitope (Theobald, et al., Proc. Natl. Acad. Sci. 92:11993-11997 (1995), Theobald, et al., J. Exp. Med., 185(5):833-841 (1997)). Four out of nine predicted p53.261 analogs were found to induce stronger analog-specific CTL responses in vivo compared to the CTL responses induced by the native peptide. More significantly, when the cross-reactivity of the CTL raised by immunization with heteroclitic analogs was analyzed, three p53.261 analogs induced CTL which responded vigorously against the native p53.26epitope. Finally, the relevance of these findings for human CTL was addressed by demonstrating that heteroclitic analogs of the MAGE3.112 epitope are immunogenic for human T cells in vitro. The resulting CTL can recognize wild-type naturally processed antigen in the form of tumor cell lines.

The studies presented herein demonstrate that heteroclicity is a global phenomenon, as heteroclitic analogs were identified for all the epitopes studied. In addition, the present application shows that it is possible to detect heteroclitic analogs both in clonal T cell populations (as has been described earlier studies) as well as in bulk T cell populations following in vivo immunization. Moreover, it is demonstrated herein that heteroclicity (both in the HLA A2.1 system as well as for other class I supermotifs) is associated with discrete structural features which allow rational prediction of heteroclicity.

The heteroclitic analogs were effective in raising bulk populations of specific T cells following in vivo immunization. Polyclonal responses that bear TCR from multiple TCR genes, are more efficacious in resolving disease states in a clinical setting. Finally, the ability to generate high precursor frequencies of CTL possessing strong cross-reactive avidity against wild-type epitope is important in instances where effective CTL responses against epitopes, normally tolerant to the immune system, are required.

In another set of experiments, Applicants identified heteroclitic analogs of the B7 superfamily epitope MAGE2.170 (shown in Table 1). Like A2 heteroclitic epitopes, heteroclitic analogs of the B7 superfamily epitope could be generated by introducing substitutions at an odd-number position in the middle of the peptide (position 7). The nature of the substitutions for the MAGE2.170 epitope were either conservative/semi-conservative (the Y→H and Y→M substitutions) or non-conservative (the Y→E, Y→G, and Y→D substitutions) compared to the native residue (Table 2). Thus, the observation that non-conservative substitutions can result in heteroclitic analogs for the MAGE2.170 CTL epitope indicate a partially overlapping substitution pattern with that observed with A2 superfamily epitopes.

Differential regulation of production of Th1 or Th2 cytokines was not observed. Instead, the present data suggested that the heteroclitic analogs increased the production of both Th1 and Th2 responses, although the magnitude and kinetics of the increase may be different. In fact, some groups (Nicholson, et al., Int. Immunol. 12(2):205-13 (2000), Parkhurst, et al., J. Immunol. 157:2539 (1996)) have recently reported such overall stimulation by peptide analogs. This is attributable to a stronger TCR signal induced by analogs, though the mechanism of such overall stimulation remains to be elucidated.

The efficacy of heteroclitic analogs in vivo using relevant tumor models or models in which tolerance to self-antigens exists is evaluated. Accordingly, it is found that immunization with heteroclitic analogs is a more effective and efficient strategy for vaccination against tumors where raising effective CTLs has so far proved to be a challenge.

### Example 2

### Screening of Peptide Analogs for Heteroclitic Activity

A. Identification of CEA.691 and MAGE3.112 Analogs Associated with Increased IFN-γ Release

Prior to screening analogs, a peptide dose titration of IFN-γ production from CTL lines was performed over a wide range of doses of wild-type peptide. 0.221A2.1 tumor cells were pulsed with varying doses of peptide then 10⁵ peptide-loaded cells were cultured with an equivalent number of murine or human CTL. After 24 hr (murine) or 48 hr (human) incubation at 37° C., levels of IFN-γ released by CTL were measured by the in situ capture ELISA assay. After determining a dose titration curve, a suboptimal peptide dose where activity against wild-type peptide was barely detectable was selected for screening the antigenicity of a panel of peptide analogs. For all of the murine and human CTL lines, this suboptimal dose ranged from 0.1-1 μg/ml. It should be noted that although murine CTL lines were generated in HLA-A2.1/K^{bxs} transgenic mice which express an HLA molecule with murine H-2 K^{b} sequences in the third domain, all responded to peptide presented on APC expressing the native HLA-A2.1 molecule.

For screening of peptide analogs, 0.221A2.1 cells were pulsed with each analog at the selected suboptimal dose and peptide-loaded APC were cultured with CTL as described above. Analogs inducing enhanced CTL responses relative to wild-type peptide were then selected for further characterization. These analogs were characterized by performing a peptide dose titration side-by-side with the wild-type epitope under identical conditions described above.

CTL lines specific for the HLA-A2.1-restricted CEA.691 and MAGE3.112 epitopes were derived by repeated in vitro restimulations of human PBMCs with peptide-loaded dendritic cells or adherent monocytes, as described in Preparation C.

A total of 117 CEA.691 and 116 MAGE3.112 analogs were generated by systematically replacing each residue with 17 different single amino acids. CEA.691 is IMIGVLVGV (SEQ ID NO:1); MAGE3.112 is KVAELVHFL (SEQ ID NO:4). The residues Cys, Trp and Met were in general avoided unless they corresponded to conservative changes. Substitutions were introduced at all positions in the peptide except at the main MHC anchor positions, position 2 and the C-terminus.

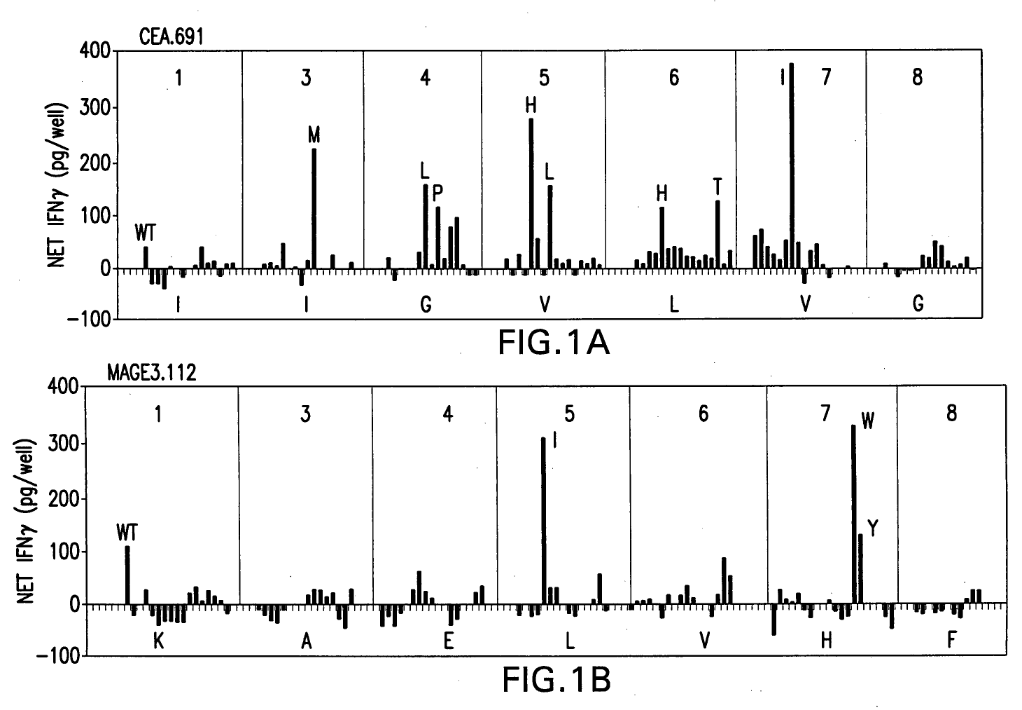

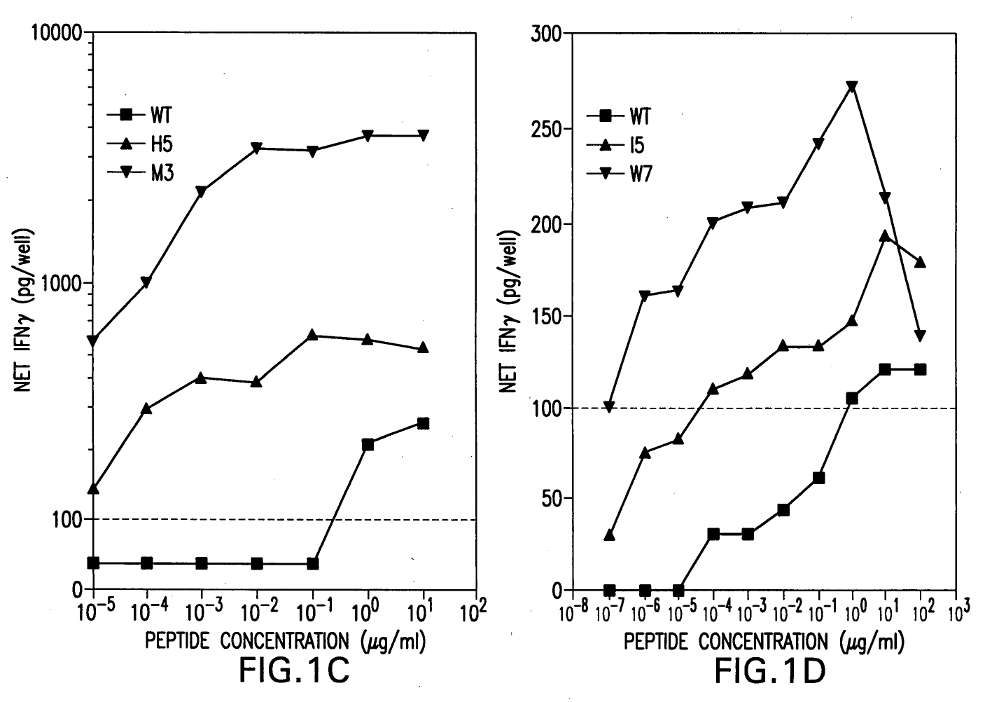

These analogs were then tested in vitro for their antigenicity. As described above, preliminary dose titration experiments for each CTL line were performed to define an antigen concentration at which IFN-γ production in response to wild-type peptide was barely detectable. This suboptimal concentration was then used subsequently for all antigenicity analysis on analog peptides for each epitope, to identify analogs associated with increased T cell stimulatory capacity. Results of such antigenicity analysis are shown in FIG. 1. As shown in FIG. 1A, the suboptimal 100 ng/ml dose the wild-type CEA.691 peptide yielded only marginal IFN-γ production (<50 pg/well). By contrast, at the same dose, several CEA.691 analogs (M3, L4, P4, H5, L5, H6, T6, and I7) induced detectable levels of IFN-γ production, in the 150 to 350 pg/well range. As shown in FIG. 1B, MAGE3.112-specific CTL line 100 ng/ml of wild-type peptide induced the release of 100 pg/ml of IFN-γ, whereas two analogs (I5 and W7) were associated with inducing IFN-γ levels of over 300 pg/well.

All analogs of CEA.691 and MAGE3.112 that stimulated IFN-γ above 100 pg/well were chosen for further characterization and a complete dose titration was carried out to identify heteroclitic analogs. Heteroclitic analogs are those that stimulate significant IFN-γ release (>100 pg/well) at 10-fold or lower peptide concentrations than wild-type peptide. For the CEA.691 epitope two different analogs, M3 (SEQ ID NO:2) and H5 (SEQ ID NO:3), were identified. As seen in FIG. 1C, for epitope CEA.691, the wild-type peptide yielded a significant detectable IFN-γ signal in the 1 to 100 μg/ml dose range, while the analogs M3 and H5 stimulated significant release with as little as 0.01 ng/ml of peptide. By these criteria, these two CEA.691 analogs are, on a molar basis, 100,000-fold more potent in terms of IFN-γ release than their unmodified wild-type counterpart.

Similarly, for the MAGE3.112 epitope two heteroclitic analogs, I5 and W7, were identified. As shown in FIG. 1D, 1 μg/ml of wild-type peptide concentration is required for significant IFN-γ release whereas 0.1 ng/ml of either I5 (SEQ ID NO:5) or W7 (SEQ ID NO:6) analogs was required to stimulate an equivalent response. This corresponds to a greater than 100,000-fold increase in biological activity compared to wild-type peptide.

In general, the modification of a wild-type class I epitope by substitution with a conservative or semi-conservative amino acid at position 3 and/or 5 and/or 7 of the epitope to generate a heteroclitic analog enhances the immune response to the corresponding wild-type epitope. The heteroclitic analogs not only induced a dose response shift, but also stimulated CTL's to produce higher levels of IFN-γ compared to wild-type peptide so that the maximal dose response (plateau) reached in response to the analog was much higher than the response obtained in response to the unmodified antigen.

### Example 3

### Identification of Additional Heteroclitic Analogs

A similar strategy was employed in the creation of heteroclitic analogs of the A3 superfamily epitope CEA.61 (HLFGYSWYK, SEQ ID NO:10) and of the A24 superfamily epitope MAGE2.156 (EYLQLVFGI, SEQ ID NO:20). Table 6 provides a summary of the heteroclitic analogs created for CEA.61 and MAGE2.156. Analogs were initially screened for IFN-γ production by responder cell lines that was greater than 2-fold over the wild-type peptide. If the responses to all of the analogs was less than 2-fold, then the analogs were screened on a case-by-case basis.

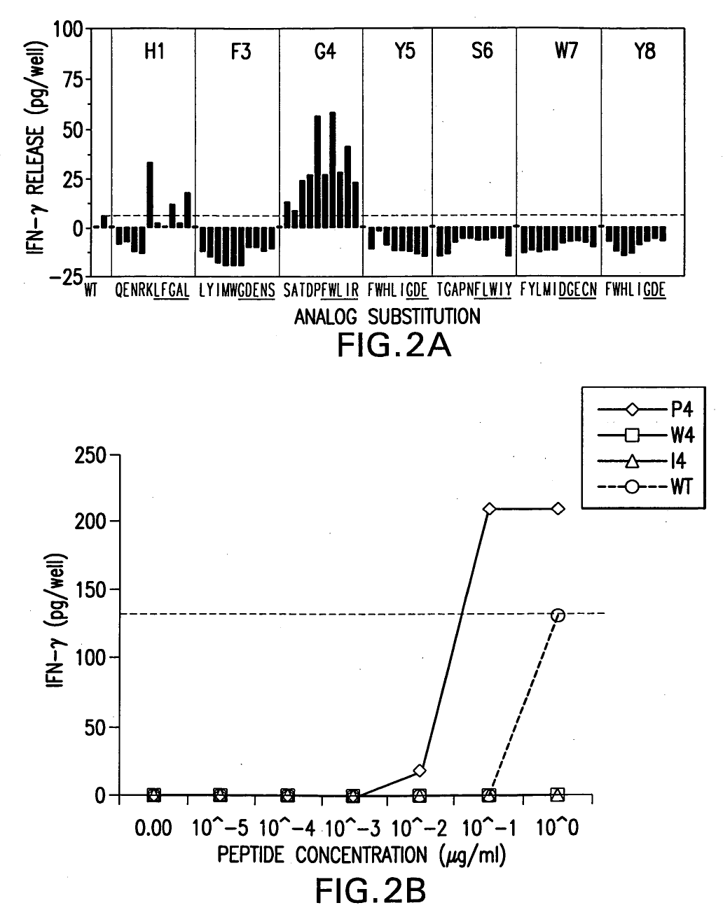

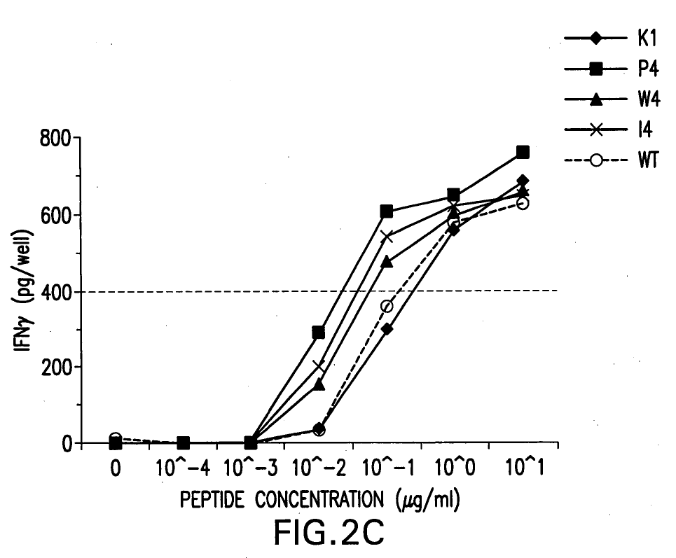

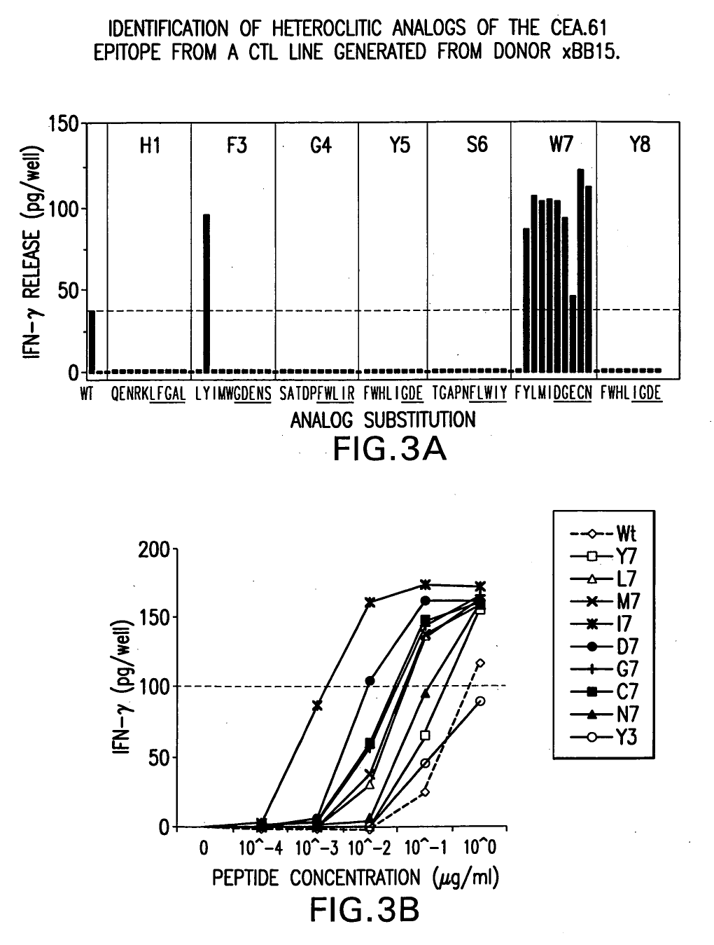

Analysis of the 67 different analogs of the CEA.61 epitope tested resulted in identification of eight heteroclitic analogs, P4 (SEQ ID NO:11), L7 (SEQ ID NO:13), M7 (SEQ ID NO:14), I7 (SEQ ID NO:15), D7 (SEQ ID NO:16), G7 (SEQ ID NO:17), C7 (SEQ ID NO:18), and N7 (SEQ ID NO:19), that stimulated IFN-γ responses at 100- to 100,000-fold lower doses than wild-type peptide (FIGS. 2B-2C and 3B); the analogs had substitutions that were conservative, semi-conservative, or non-conservative in nature occurring at both even and odd-numbered positions in the peptide (positions 4 and 7).

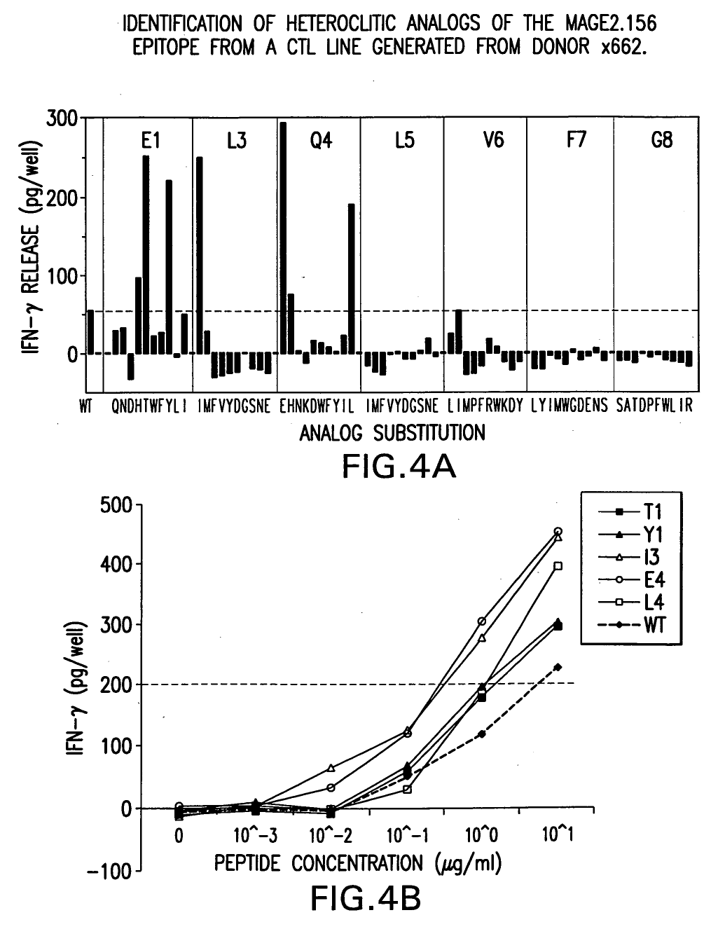

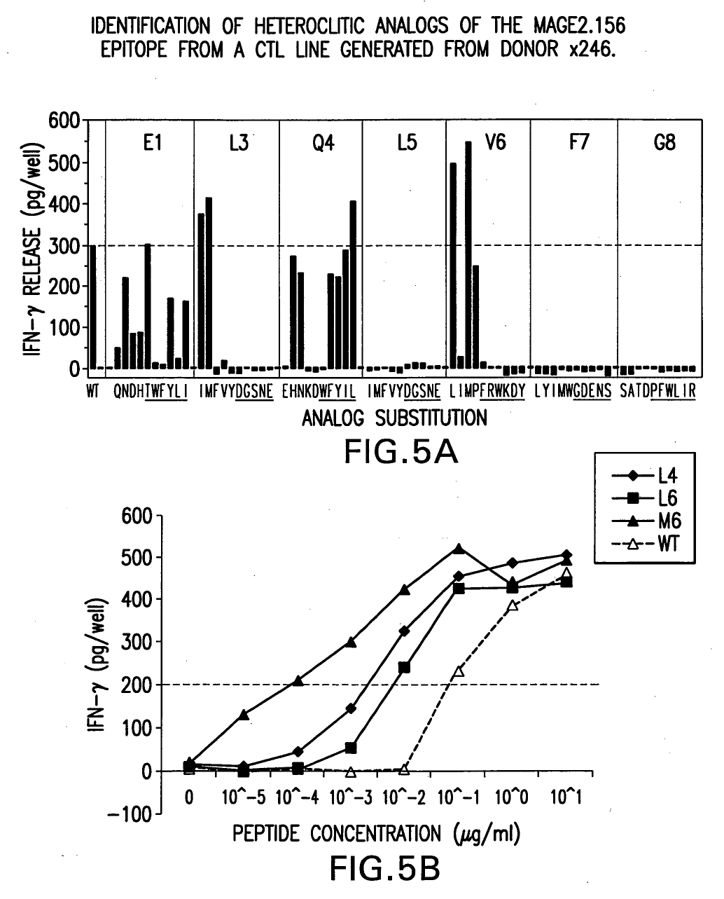

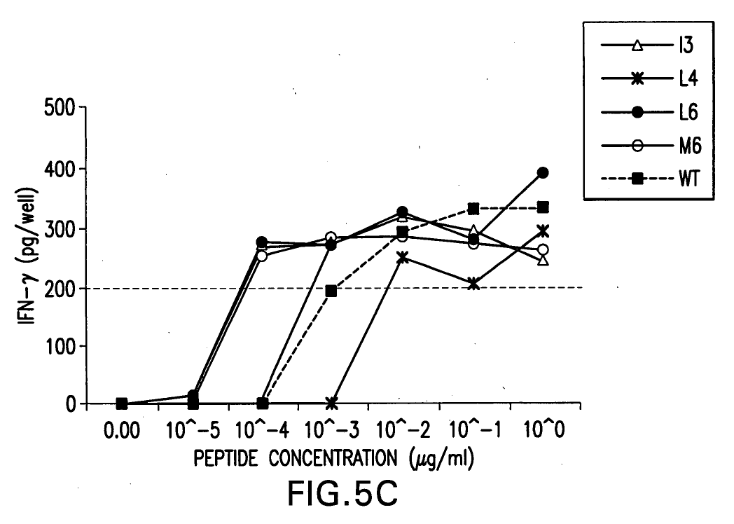

Analysis of the 71 different analogs of the MAGE2.156 epitope tested resulted in identification of five heteroclitic analogs, 13 (SEQ ID NO: 21) E4 (SEQ ID NO:22), L4 (SEQ ID NO:23), M6 (SEQ ID NO:24), and L6 (SEQ ID NO:25), that stimulated IFN-γ responses at 100- to 100,000-fold lower doses than wild-type peptide (FIGS. 4B and 5B); these analogs had substitutions that were conservative or non-conservative in nature occurring at both even and odd-numbered positions in the peptide (positions 3, 4, and 6).

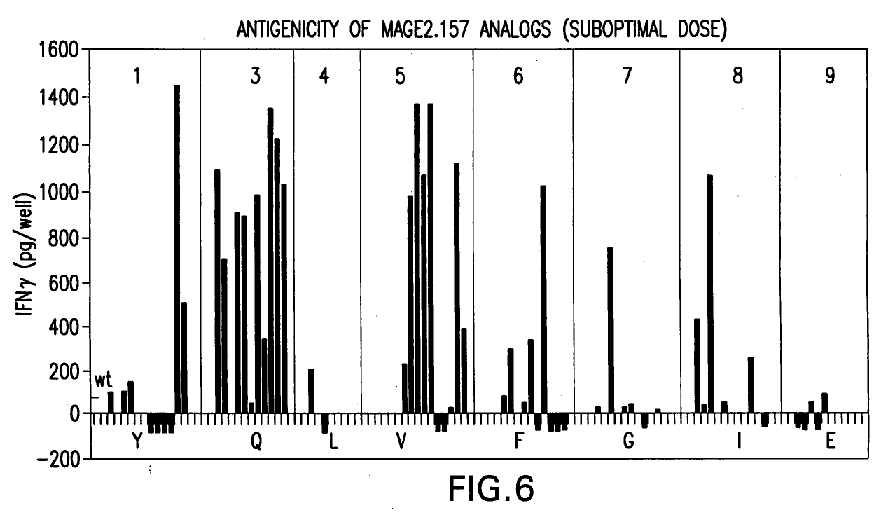

Additionally, a panel of 85 different analogs was synthesized which included five conservative and five non-conservative amino acid substitutions at epitope positions 3, 5, 7 in each of the three epitopes, as well as at epitope positions 1, 4, 6, using the amino acid conservancy assignments described in the Preparation B and in Table 2. These analogs were tested for heteroclicity using murine CTL lines generated in HLA-A2.1/K^{bxs} transgenic mice and following an experimental strategy similar to the one described in Example 1 for the CEA.691 and MAGE3.112 epitopes. Murine CTL lines derived from HLA transgenic mice were used instead of human CTL lines due to technical ease associated with generating and maintaining mouse lines. The results for MAGE2.157 are shown in FIG. 6.

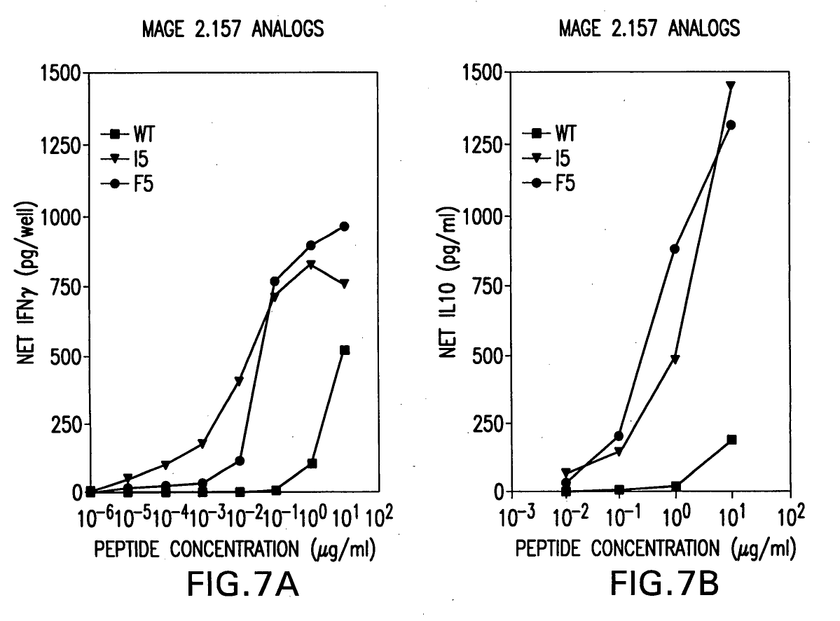

Analysis of the 85 different analogs of the MAGE2.157 epitope tested resulted in identification of two heteroclitic analogs, 15 (SEQ ID NO: 8) and F5 (SEQ ID NO: 9), that stimulated IFN-γ responses at 100- to 100,000-fold lower doses than wild-type peptide (FIG. 7A); both of these analogs had substitutions that were conservative or semi-conservative in nature occurring at an odd-numbered position in the center of the peptide (position 5).

Thus, data obtained from 85 analogs for the tumor epitope MAGE2.157 was consistent with the analysis of the MAGE3.112 and CEA.691 epitopes as set forth in Example 1.

### Example 4

### Lymphokine Profile Induced by Heteroclitic Analogs

Heteroclitic analogs have been shown previously to differentially activate cytokine production from T cells whereby some analogs specifically activate T cells to produce Th1 cytokines whereas others preferentially activate the production of Th2 cytokines. To investigate the pattern of lymphokine release associated with the heteroclitic analogs of the invention, the production of the Th2 cytokine IL-10 from CTL lines was compared to the production of IFN-γ. Representative data using the MAGE2.157 epitope is shown in FIGS. 7A and 7B.

FIGS. 7A and 7B show the lymphokine profile induced by MAGE2.157 analogs. IFN-γ (A) and IL-10 (B) produced by MAGE2.157-specific CTLs in response to 0.221A2.1 targets pulsed with analogs I5 or F5, or wild-type (WT) peptide was measured over several different doses. Dotted lines indicate significant levels of IFN-γ (100 pg/well) or IL10 (50 pg/ml). As seen in FIG. 7A, the F5 and I5 analogs of MAGE2.157 induced significant levels of IFN-γ production at 100-fold or 10,000-fold lower concentrations than wild-type peptide respectively. Moreover, the same analogs also induced significant IL-10 production at 10-fold or 100-fold lower peptide concentrations than wild-type peptide.

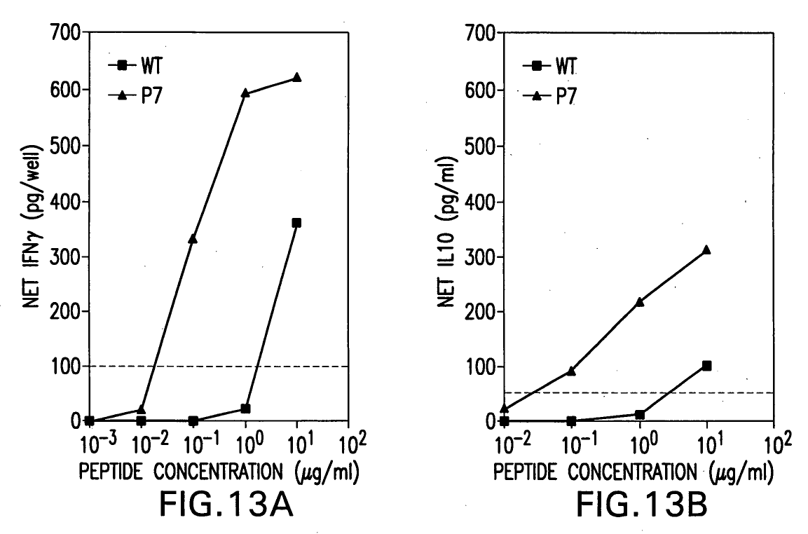

Data from another epitope, HBV Pol.455, depicting the same trend are shown in FIGS. 13A and 13B. IFNγ (A) or IL10 (B) released by HBV Pol.455 CTL's in response to analog P7 or wildtype (WT) peptide over several different peptide doses are shown. Once again, the P7 analog of HBV Pol.455 induced significant levels of IFNγ (FIG. 13A) and IL10 (FIG. 13B) at 100-fold lower peptide concentrations than wildtype peptide. Taken together the data summarizing all the heteroclitic analogs tested for induction of Th2 cytokines (Table 7) indicates that most heteroclitic analogs stimulate increased production of both of Th1 and Th2 cytokines.

### Example 5

### HLA-A2.1 Binding Affinity of Heteroclitic Analogs

To verify that the enhanced recognition by CTL lines observed was not due to a fortuitous increase in MHC binding capacity of the analog epitope, the MHC binding affinity of all heteroclitic analogs was measured in vitro utilizing purified HLA-A2.1 molecules, and compared to their unmodified wild-type counterparts as described in Preparation D.

As summarized in Table 6, three analogs (MAGE3.112 W7, HIV Pol.476 H3, and HIV Pol.476 L3) bound to HLA-A2.1 with four-fold or higher affinity than wild-type peptide and two analogs bound with lower affinity (MAGE2.157 I5, MAGE2.157 F5). The four remaining heteroclitic analogs, MAGE3.112 I5, CEA.691 M3, CEA.691 H5, and HBV Pol.455 P7, were associated with little or no change in HLA-A2.1 binding capacity. Collectively these data suggest a lack of correlation between increased binding and heteroclicity.

### Example 6

### Heteroclitic Analogs Induce Human CTL Capable of Recognizing Tumor Cells in Vitro

Immunogenicity of heteroclitic analogs of MAGE3.112 was also tested by inducing primary CTL from PBMC, as described in Preparation C, against either the MAGE3.112 peptide or the I5 and W7 analogs of this epitope. After two rounds of in vitro stimulation, PBMC cultures in 48-wells were scored positive for CTL induction if the net IFN-γ production was >100 pg/well and production was at least two-fold above background, after stimulating with 0.221-A2.1 APC in the presence or absence of peptide.

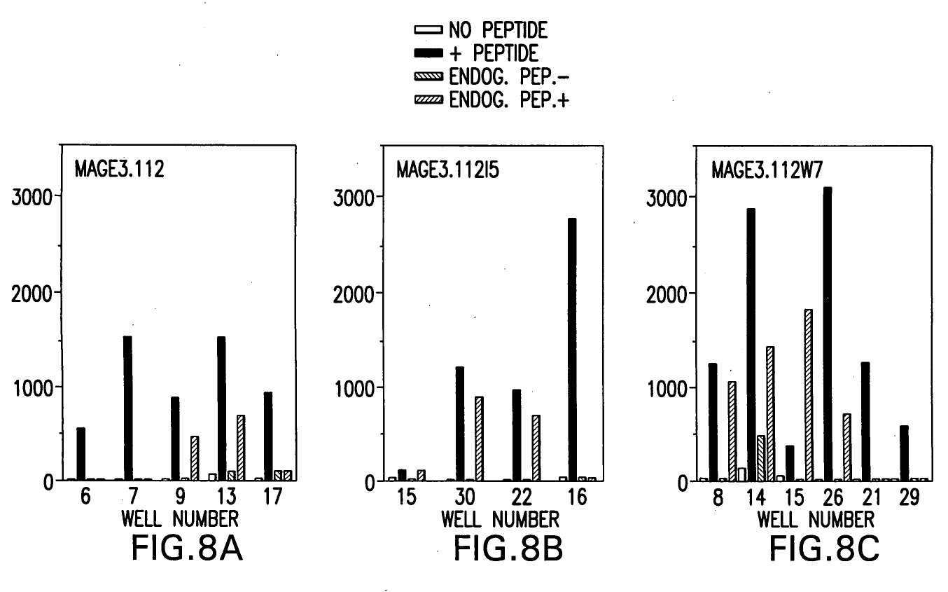

To underline the physiologic relevance of our observations to human tumor antigens, we examined whether heteroclitic analogs of the MAGE3.112 epitope could induce human CTL's in a primary in vitro induction system. Fresh naïve human PBMC from normal donors were stimulated repetitively in vitro with either wild-type or analogs as described previously (Kawashima, I., et al., Human Immunol. (1998) 59:1). Peptide-specific CTL responses were detected in cultures stimulated with either wild-type peptide (FIG. 8A) or the I5 (FIG. 8B) and W7 analogs (FIG. 8C). Briefly, 0.221A2.1 cells were pulsed overnight with 10 μg/ml of WT peptide (FIG. 8A), the I5 (FIG. 8B) analog, or the W7 analog (FIG. 8C). IFN-γ production by CTL's growing in individual wells from a 48-well plate were tested against 0.221A2.1 cells in the presence or absence of peptide, or against the endogenous epitope-negative 888mel and the endogenous epitope-positive 624mel tumor cell lines. Only wells showing a positive peptide-specific CTL response are shown.

More importantly cultures induced with these analogs recognized the 624mel tumor cell line that endogenously processes and presents the wild-type sequence. This demonstrates that heteroclitic analogs can induce physiologically relevant human CTL's that recognize endogenously-generated wild-type peptide presented by tumor cells and that the phenomenon is relevant in both human and in transgenic mouse systems.

### Example 7

### Identification of Heteroclitic Analogs of a B7 Superfamily CTL Epitope, MAGE2.170

To better define the application of the invention to HLA Supertype families other than HLA-A2, analogs of the B7 superfamily epitope MAGE2.170 (sequence VPISHLYIL) (SEQ ID NO:43) were synthesized and screened in a fashion similar to that described previously for A2 superfamily epitopes. A panel of analogs of the MAGE2.170 epitope consisting of conservative/semi-conservative and non-conservative substitutions at every non-anchor position were screened at two suboptimal peptide doses using a human CTL line generated against the wild-type epitope. As previously described, this screening assay served to identify any potentially heteroclitic analogs that induce stronger CTL responses compared to wild-type peptide.

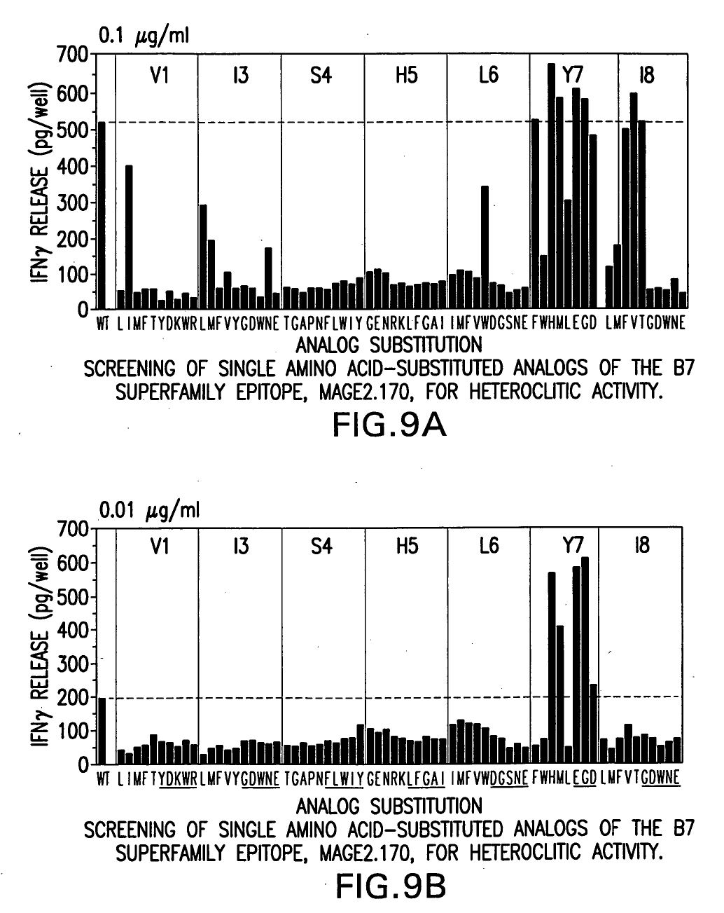

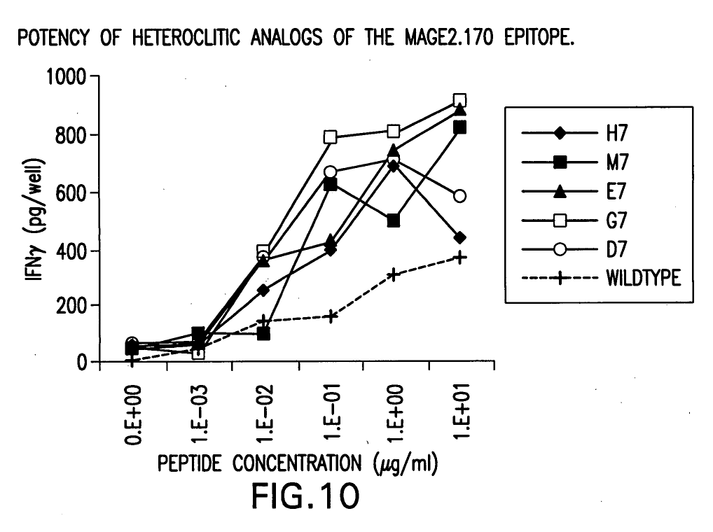

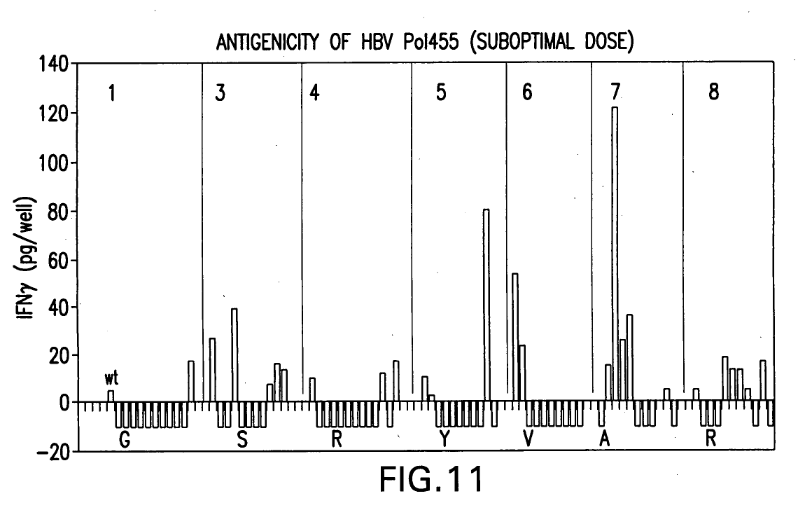

As shown in FIGS. 9A-9B, analogs substituted at position 7 with either a H, M, E, G, or D residue stimulated IFN-γ responses that were greater than the wild-type peptide when tested at the 0.01 μg/ml dose. When the stimulatory capacity of these five analogs were further analyzed in a peptide dose titration using the same wild-type epitope-specific CTL line, all of them demonstrated strong heteroclitic activity inasmuch as they all stimulated an equivalent level of IFN-γ production (e.g. 200 pg/well) at >10-fold lower doses compared to the wild-type epitope, and the magnitude of response stimulated by the analogs was >2-fold greater than wild-type epitope at several peptide doses (FIG. 10).

To determine whether the heteroclitic activity of MAGE2.170 analogs was correlated with an increase or decrease in MHC binding activity, the binding affinity of the H7, M7, E7, G7, and D7 analogs to purified HLA-B7 molecules was determined relative to the wild-type epitope. Results shown in Table 8 indicate that there was no correlation between MHC binding of the analogs and heteroclicity inasmuch as 4 of the 5 MAGE2.170 analogs demonstrated binding affinities within a two-fold range of the wild-type peptide. The fifth epitope, MAGE2.170 D7, demonstrated a >100-fold decrease in binding compared to the wild-type peptide, therefore an enhancement in MHC binding could not account for the heteroclitic activity observed with this analog.

In summary, these results indicate that heteroclitic analogs can be generated from a B7 superfamily epitope by introducing single amino acid substitutions and that the substitution pattern showed similarity and differences with A2 heteroclitic epitopes. Like A2 heteroclitic epitopes, heteroclitic analogs of the B7 superfamily epitope MAGE2.170 could be generated by introducing substitutions at an odd-number position in the middle of the peptide (position 7). The nature of the substitutions for the MAGE2.170 epitope was either conservative/semi-conservative (the Y→H and Y→M substitutions) or non-conservative (the Y→E, Y→G, and Y→D substitutions) compared to the native residue (Table 8). Thus, the observation that non-conservative substitutions can result in heteroclitic analogs for the MAGE2.170 CTL epitope indicate a partially overlapping substitution pattern than that observed with A2 superfamily epitopes.

### Example 8

### Synthesis and Analysis of Heteroclitic Analogs Derived from the HLA-B7 Supermotif on HLA B7 Superfamily Members

To further validate the heteroclitic substitution rules, additional studies are carried out with heteroclitic analogs derived from a peptide bearing a sequence within the HLA-B7 supermotif. For example, the analogs can be tested for in vivo immunogenicity.

For this study, the HLA-B7 supermotif bearing peptide, APRTLVYLL (SEQ ID NO:36) epitope is chosen and synthesized. A panel of analogs consisting of three conservative/semiconservative substitutions at positions 3, 5 and 7 of the 9-mer peptide, are tested for immunogenicity in HLA-B*0702/K^{b} transgenic mice. The panel includes APETLVYLL (SEQ ID NO:37), APRTWVYLL (SEQ ID NO:38), and APRTLVPLL (SEQ ID NO:39), corresponding to a semi-conservative change is the third, fifth and seventh position, respectively.

CTLs from the mice immunized with the above-described analogs are tested for induction of at least 100 pg/well of IFN-γ production. This IFN-γ production will typically occur at much lower peptide concentrations than those induced and restimulated with wild-type peptide (e.g., APRTLVYLL) (SEQ ID NO:36). These results will indicate that our predicted heteroclitic analogs are more potent at inducing higher avidity CTL than wild-type peptide itself.

Typically, CTLs obtained from animals immunized and restimulated with a wild-type peptide will induce 100 pg/well IFN-γ at peptide doses of 5-10 μg/ml, whereas CTLs obtained from animals immunized with the above-described analogs, and stimulated and tested in vitro with wild-type peptide, require 10-fold, 100-fold or even 1000-fold lower doses of wild-type peptide respectively, to induce 100 pg/well of IFN-γ.

To further validate the heteroclitic substitution rules for other HLA molecules with the B7 superfamily, the peptides APETLVYLL(SEQ ID NO:37), APRTWVYLL (SEQ ID NO:38) and APRTLVPLL (SEQ ID NO:39) are tested for in vivo immunogenicity in transgenic mice expressing one of the following human HLA molecules: B*0702, B*0703, B*0704, B*0705, B*1508, B*3501, B*3502, B*3503, B*3503, B*3504, B*3505, B*3506, B*3507, B*3508, B*5101, B*5102, B*5103, B*5104, B*5105, B*5301, B*5401, B*5501, B*5502, B*5601, B*5602, B*6701 and B*7801.

CTLs from the mice immunized with the above-described analogs are tested for induction of at least 100 pg/well of IFN-γ production. This IFN-γ production will typically occur at much lower peptide concentrations than those induced and restimulated with wild-type peptide (e.g., APRTLVYLL) (SEQ ID NO:36). These results will indicate that our predicted heteroclitic analogs are more potent at inducing higher avidity CTL than wild-type peptide itself.

Typically, CTLs obtained from animals immunized and restimulated with a wild-type peptide will induce 100 pg/well IFN-γ at peptide doses of 5-10 μg/ml, whereas CTLs obtained from animals immunized with the above-described analogs, and stimulated and tested in vitro with wild-type peptide, require 10-fold, 100-fold or even 1000-fold lower doses of wild-type peptide respectively, to induce 100 pg/well of IFN-γ.

Precursor Frequency Analysis Using ELISPOT Assays

To confirm that cross-reactive CTL against wild-type peptide are generated in mice immunized with analogs, CD8⁺ cells are isolated from spleens immunized with analogs or wild-type peptide without farther CTL expansion in vitro. From this material, the precursor frequency of CTL reactive against either wild-type or analog using ELISPOT assay is determined. The precursor frequencies of wild-type peptide reactive CTLs are typically much lower than the precurser frequencies of the analogs.

Heteroclitic Analogs can Induce Human CTL Capable of Recognizing Epitopes in Vitro

Heteroclitic analogs can be analyzed for induction of CTLs in a primary in vitro induction system. Fresh naive human PBMC from normal donors are stimulated repetitively in vitro, with either wild-type or analogs, in 48 well plates as described previously. Peptide specific CTL responses are then detected in cultures stimulated with either a wild-type peptide or a heteroclitic analog. Cultures induced with these analogs can recognize targets that are endogenously processed and present the wild-type sequence. This demonstrates that heteroclitic analogs can induce physiologically relevant human CTLs that recognize endogenously generated wild-type peptide expressed on cells and that the phenomenon is relevant in both human and in transgenic mouse systems.

### Example 9

### Synthesis and Analysis of Heteroclitic Analogs Derived from the HLA-A3 Supermotif on HLA-A3 Superfamily Members

To further validate the heteroclitic substitution rules, additional studies are carried out with heteroclitic analogs derived from a peptide bearing a sequence within the HLA-A3 supermotif For example, the analogs can be tested for in vivo immunogenicity.

For this study, the HLA-A3 supermotif bearing peptide, KVFPYALINK (SEQ ID NO:33) epitope is chosen and synthesized. A panel of analogs of SEQ ID NO:33 consisting of three conservative/semiconservative substitutions at positions 3, 5 and 7 of the 9-mer peptide, are tested for immunogenicity in HLA-A*3101/K^{b} transgenic mice. The panel includes KVHPYALINK (SEQ ID NO:40), KVFPQALINK (SEQ ID NO:41) and KVFPYAKINK (SEQ ID NO:42), corresponding to a semi-conservative change in the third, fifth and seventh position, respectively.

CTLs from the mice immunized with the above-described analogs are tested for induction of at least 100 pg/well of IFN-γ production. This IFN-γ production typically occurs at much lower peptide concentrations than those induced and restimulated with wild-type peptide (e.g., KVFPYALINK) (SEQ ID NO:33). These results indicate that our predicted heteroclitic analogs are more potent at inducing higher avidity CTL against wild-type than wild-type peptide itself.

Typically, CTLs obtained from animals immunized and restimulated with a wild-type peptide induce 100 pg/well IFN-γ at peptide doses of 5-10 μg/ml, whereas CTLs obtained from animals immunized with the above-described analogs, and stimulated and tested in vitro with wild-type peptide, require 10-fold, 100-fold or even 1000-fold lower doses of wild-type peptide respectively, to induce 100 pg/well of IFN-γ.

To further validate the heteroclitic substitution rules for other HLA molecules with the A3 superfamily, the peptides KVHPYALINK(SEQ ID NO:40), KVFPQALINK (SEQ ID NO:41) and KVFPYAKINK (SEQ ID NO:42) are tested for in vivo immunogenicity in transgenic mice expressing one of the following human HLA molecules: A*0301, A*1101, A*3101, A*3301 and A*6801.

CTLs from the mice immunized with the above-described analogs are tested for induction of at least 100 pg/well of IFN-γ production. This IFN-γ production typically occurs at much lower peptide concentrations than those induced and restimulated with wild-type peptide (e.g., KVFPYALINK) (SEQ ID NO:33). These results will indicate that our predicted heteroclitic analogs are more potent at inducing higher avidity CTL than wild-type peptide itself.

Typically, CTLs obtained from animals immunized and restimulated with a wild-type peptide induce 100 pg/well IFN-γ at peptide doses of 5-10 μg/ml, whereas CTLs obtained from animals immunized with the above-described analogs, and stimulated and tested in vitro with wild-type peptide, require 10-fold, 100-fold or even 1000-fold lower doses of wild-type peptide respectively, to induce 100 pg/well of IFN-γ.

Precursor Frequency Analysis Using ELISPOT Assays

To confirm that cross-reactive CTL against wild-type peptide are generated in mice immunized with analogs, CD8⁺ cells are isolated from spleens immunized with analogs or wild-type peptide without further CTL expansion in vitro. From this material, the precursor frequency of CTL reactive against either wild-type or analog using ELISPOT assay is determined. The precursor frequencies of wild-type peptide reactive CTLs are typically much lower than the precurser frequencies of the analogs.

Heteroclitic Analogs can Induce Human CTL Capable of Recognizing Epitopes in Vitro

Heteroclitic analogs are analyzed for induction of CTLs in a primary in vitro induction system. Fresh naive human PBMC from normal donors are stimulated repetitively in vitro, with either wild-type or analogs, in 48 well plates as described previously. Peptide specific CTL responses are then detected in cultures stimulated with either a wild-type peptide or a heteroclitic analog. Cultures induced with these analogs recognize targets that are endogenously processed and present the wild-type sequence. This demonstrates that heteroclitic analogs induce physiologically relevant human CTLs that recognize endogenously generated wild-type peptide expressed on cells and that the phenomenon is relevant in both human and in transgenic mouse systems.

### Example 10

### Identification of Additional Heteroclitic Analogs

Three additional A2.1-restricted epitopes, the MAGE2.157 YLQLVFGIEV, SEQ ID NO: 7 tumor epitope, and two epitopes from viral antigens, HBV Pol.455, GLSRYVARL (SEQ ID NO: 55) and HIV Pol.476 ILKEPVHGV (SEQ ID NO: 57), were analyzed. All of these epitopes have previously been shown to be immunogenic for CTL.

A panel of 240 different analogs was synthesized which included five conservative and five non-conservative amino acid substitutions at epitope positions 3, 5, 7 in each of the three epitopes, as well as at epitope positions 1, 4, 6, using the amino acid conservancy assignments described in the Preparation B and in Table 2. These analogs were tested for heteroclicity using murine CTL lines generated in HLA-A2.1/K^{bxs} transgenic mice and following an experimental strategy similar to the one described in Example 1 for the CEA.691 and MAGE3.112 epitopes. Murine CTL lines derived from HLA transgenic mice were used instead of human CTL lines due to technical ease associated with generating and maintaining mouse lines.

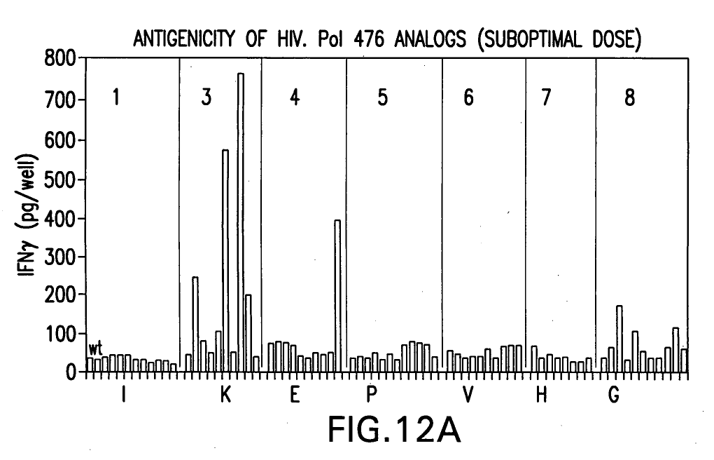

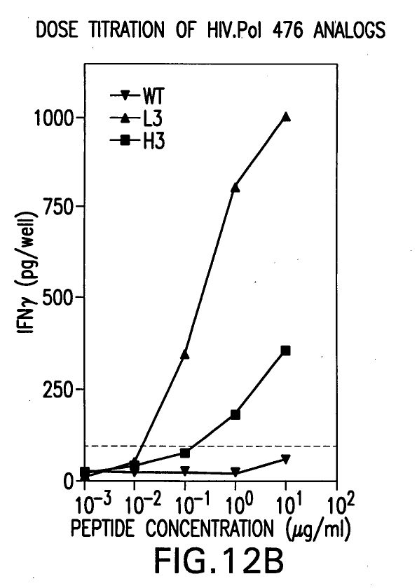

The results are shown in FIG. 6 (AGE2.157), 11 (HBV Pol.455), and 12A (HIV Pol.476) with a corresponding dose titration profile for HIV Pol.476 in FIG. 12B.

Analysis of a total of 85 different analogs of the MAGE2.157 epitope tested resulted in identification of two heteroclitic analogs, I5 (SEQ ID) NO: 8) and F5 (SEQ ID NO: 9), that stimulated IFNγ responses at 100- to 100,000-fold lower doses than wildtype peptide (Table 1); both of these analogs had substitutions that were conservative or semi-conservative in nature occurring at an odd-numbered position in the center of the peptide (position 5).

For the HIV Pol.476 epitope, out of 78 different analogs screened, two were identified as having heteroclitic activity (H3 (SEQ ID NO: 58) and L3 (SEQ ID NO: 59)) (Table 1); both analogs carried either a conservative or semi-conservative substitution at an odd-numbered position in the center of the peptide. one heteroclitic analog of HIV Pol.455 epitope out of 77 tested was identified; this analog had a conservative substitution (P) at position 7 of the peptide (SEQ ID NO: 56) (Table 1). An additional HIV Pol.476 analog is prepared and tested (ILIEPVHGV) (SEQ ID NO: 67).

Thus, data obtained from 240 analogs for three additional epitopes of tumor and viral origin (MAGE2.157, HIV Pol.476, and HBV Pol.455), were consistent with the analysis of the MAGE3.112 and CEA.691 epitopes as set forth in Example 1.

Heteroclicity analysis was also performed on two p53 epitopes. One epitope, p53.149M2, SMPPPGTRV (SEQ ID NO: 49) represents a fixed anchor analog of a human p53 epitope having a methionine residue substitution which enhances MHC binding. The second epitope, p53 Mu.184, GLAPPQHLIRV (SEQ ID NO: 52) has a sequence that is completely conserved between mice and humans (Theobald, et al., 92(26):11993 (1995)).

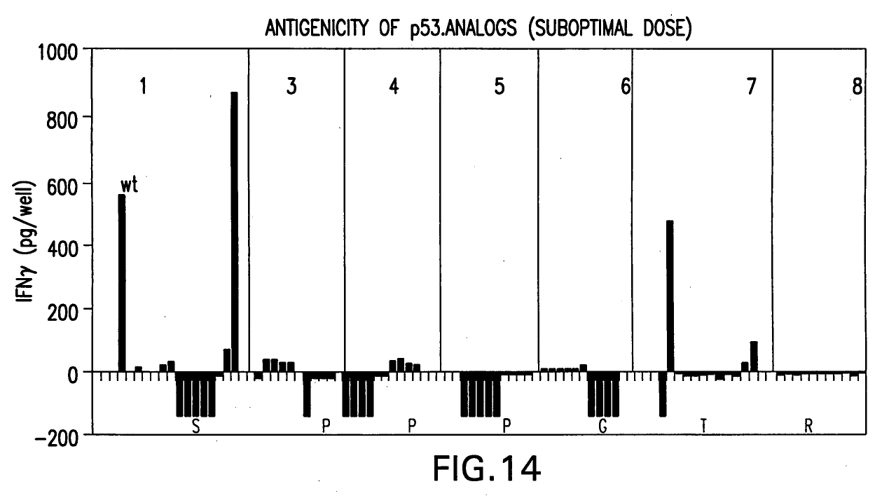

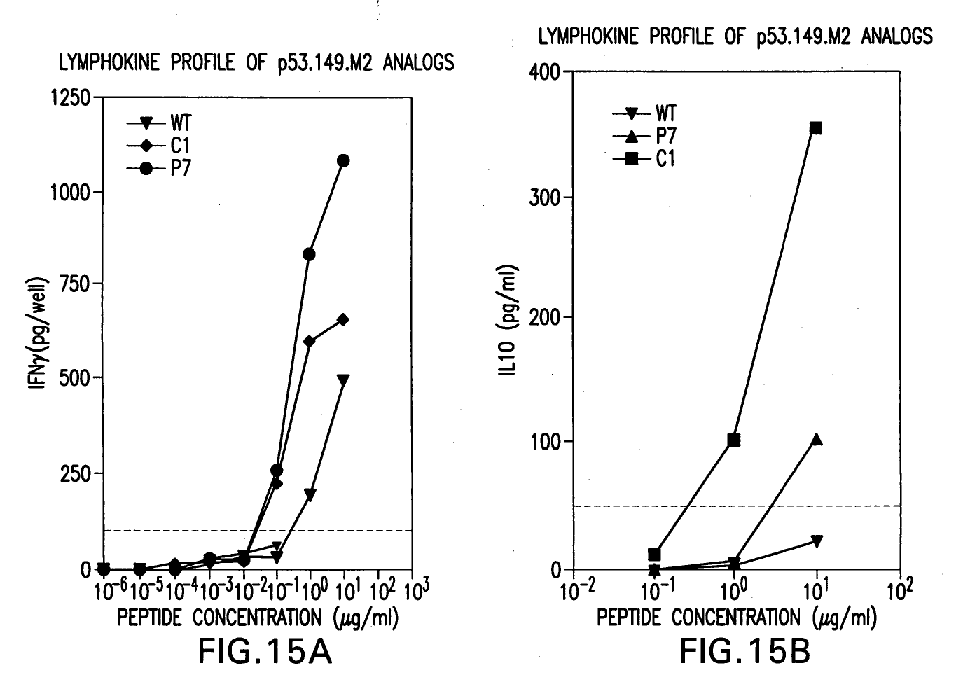

Dose titration analysis performed on the p53.149M2 revealed optimal and suboptimal responses at 1 μg/ml and 0.1 μg/ml dose range. A panel of 76 analogs for p53.149M2 (five conservative and five non-conservative substitutions at each position) was screened and only two analogs, C1 (SEQ ID NO: 50) and P7 (SEQ ID NO: 51), were identified both giving IFNγ release of 100 pg/well at a suboptimal dose, FIG. 14. On further analysis, both analogs induced significant IFNγ production at 10-fold lower concentrations than wildtype peptide. In addition, the C1 analog also induced significant IL10 levels at 100-fold lower peptide concentrations, FIG. 15A-15B.

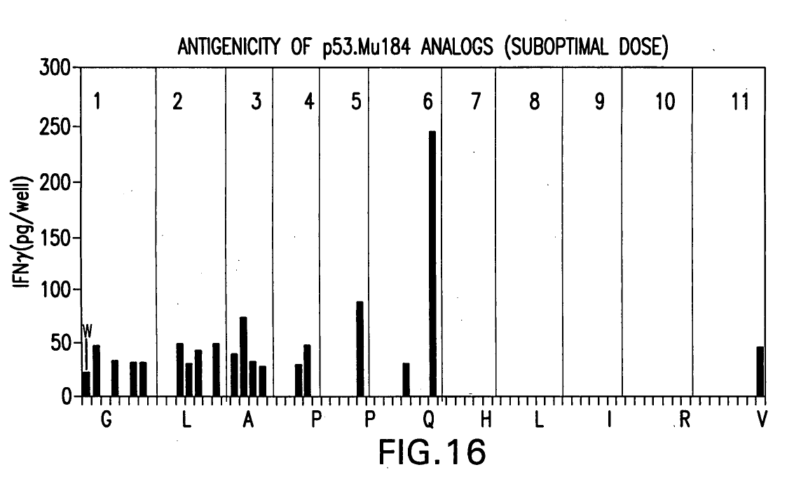

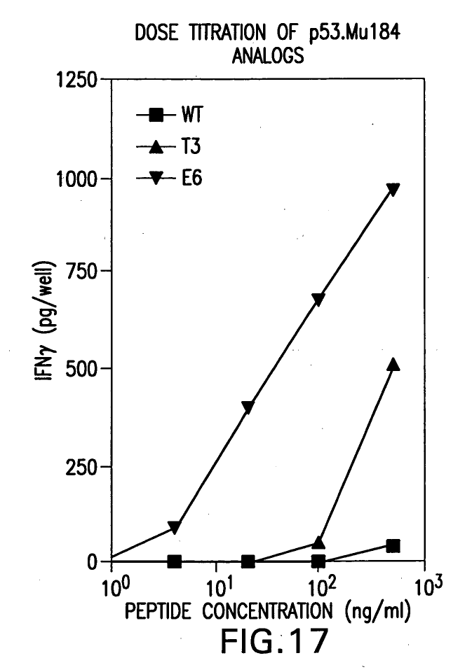

For the p53mu.184 epitope optimal and suboptimal levels of peptide were determined to be 500 ng/ml and 10 ng/ml respectively after performing a dose titration analysis. A panel of 63 conservative and semi-conservative substitution analogs were tested for immunogenicity. Two analogs with enhanced immunogenicity were found—T3 (SEQ ID NO: 53) and T3,E6 (SEQ ID NO: 54). See FIGS. 16 and 17.

### Example 11

### Prediction and Immunogenicity of Analogs for the Murine p53.261 Epitope

To test for immunogenicity in vivo, the HLA-A2.1-restricted murine p53.261 epitope was used since CTL responses against this epitope have been shown to be partially tolerized in HLA-A2.1/K^{b} transgenic mice. This permits analysis of the capacity of predicted heteroclitic analogs to break T cell tolerance in vivo. Although heteroclitic analogs heretofore have been detected through in vitro screening with CTL lines raised against wildtype epitopes, we reasoned that analogs identified by the substitution rules could potentially induce CTL in vivo that were heteroclitic against the wildtype epitope, an application of interest for designing vaccines against tolerant tumor-associated epitopes.

Immunogenicity for the p53.261 predicted analogs were tested in HLA-A2.1/K^{bxd} transgenic mice by co-immunizing mice with 50 μg of the p53.261 epitope (LLGRDSFEV) (SEQ ID NO:60) or its predicted analogs and 140 μg of HBV Core. 128 helper epitope in IFA. Eleven days later, primed spleen cells were harvested and cultured in vitro with irradiated syngeneic LPS-activated spleen cells that had been pulsed with 10 μg/ml of peptide. After 10 days of culture, CTL were restimulated with peptide-pulsed LPS blasts in the presence of Con A-conditioned media as a source of IL2 (Ishioka, G., et al., J. Immunol. (1999) 162:3915). Spleen cells from mice immunized with the predicted analogs were stimulated in vitro against both wildtype peptide (to determine the cross-reactivity, avidity and precursor frequency of CTL's that respond to wildtype antigen) and the respective immunizing analog (to determine avidity and precursor frequency of CTL's responding to the analog). All short-term, bulk populations of CTL were tested for peptide specificity by the IFNγ in situ ELISA assay 5 days after the second restimulation in vitro, using Jurkat-A2.1 tumor cells as APC. Alternatively, CTL responses were performed on freshly isolated spleen cells from immunized animals using the Elispot assay.

A panel of nine analogs of the p53.261 epitope consisting of three conservative or semi-conservative substitutions at positions 3, 5, and 7 of the 9-mer peptide was tested for immunogenicity in HLA-A2.1/K^{bxd} transgenic mice. Immunization of mice with each of the nine analogs and in vitro expansion of primed splenocytes with the respective immunizing analog resulted in identification of six analogs (L7, D3, H7, H3, N5, G5) that gave CTL responses characterized by IFNγ production of 100 pg/well at much lower peptide concentrations compared to CTL induced in vivo and expanded in vitro with wildtype peptide.

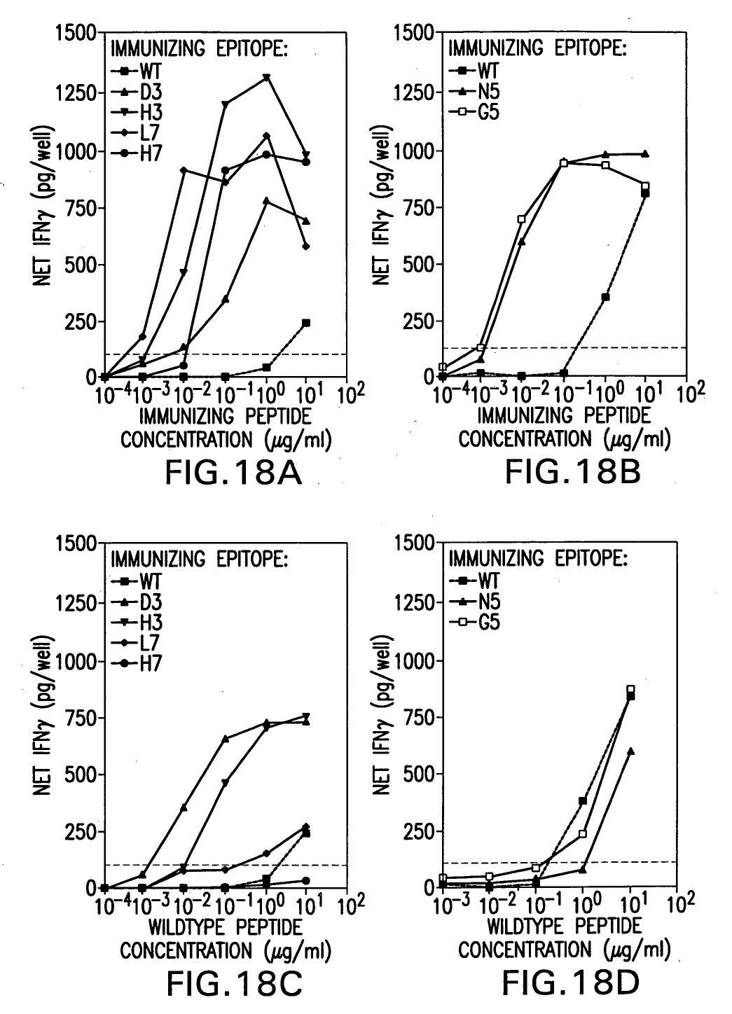

Spleen cells from mice immunized with either WT peptide or the indicated analogs were stimulated in vitro with the corresponding immunizing peptide (FIGS. 18A, B) or with WT peptide (FIGS. 18C, D). IFNγ release by these CTL's was then measured over a dose range against targets pulsed with the immunizing peptide (FIGS. 18A, B) or with WT peptide (FIGS. 18C, D). IFNγ release at 100 pg/well is shown as a dotted line. These results indicate that a significant percentage of the analogs induce CTL of a higher avidity than those induced by wildtype peptide itself.

The cross-reactivity of CTL primed with these heteroclitic analogs against wildtype peptide is shown in FIG. 18C and FIG. 18D. While CTL's obtained from animals immunized and restimulated with wildtype peptide induced 100 pg/well IFNγ at peptide doses between 0.1-10 μg/ml, CTL's obtained from animals immunized with analogs L7, H3, and D3, and stimulated and tested in vitro with wildtype peptide, required 10-, 100-, or 1000-fold lower doses of wildtype peptide respectively, to induce 100 pg/well of IFNγ (FIG. 18C). This suggests that in three out of six cases the predicted heteroclitic analogs were 10-1000-fold more active/potent at inducing CTL's reactive to wildtype peptide in situations where partial CTL tolerance to wildtype antigen exists.

### Example 12

### Cross Reactivity with Wildtype

The cross-reactivity of CTL induced by the D3 and H3 analogs were also tested against the wildtype epitope naturally processed by a p53-expressing Meth A tumor cell clone transfected with HLA-A2.1/K^{b}; it was found that CTL generated by p53.261 analogs that are heteroclitic for wildtype epitope respond to endogenously-processed p53.261 epitope presented by Meth A/A2.1K^{b} tumor cells.

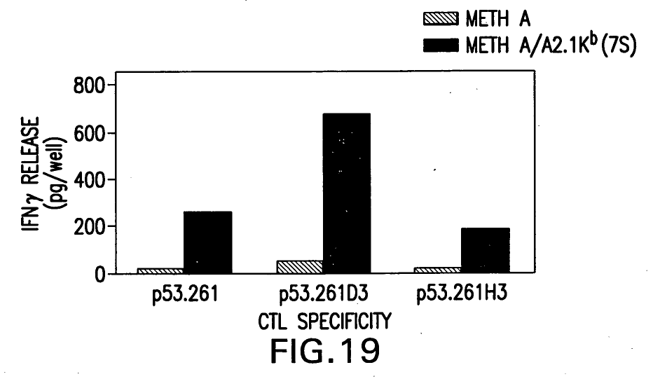

The CTL population (10⁵/well) were cultured with 2.5×10⁴ Meth A tumor cells or with a Meth A clone transfected with HLA-A2.1/K^{b} and IFNγ release was measured by the in situ ELISA assay. As shown in FIG. 19, CTL lines raised against both D3 and H3 analogs of the p53.261 epitope responded to the endogenous epitope expressed by a Meth A/A2.1K^{b} tumor cell clone but not to the parental HLA-A2.1-negative Meth A tumor cell line.

### Example 13

### Precursor Frequency Analysis Using Elispot Assays

To confirm that cross-reactive CTL against wildtype peptide are generated in mice immunized with analogs CD8⁺ cells were isolated from spleen cells of mice immunized with analogs or wildtype peptide, without further CTL expansion in vitro and the precursor frequency of CTL reactive against either wildtype or analog was determined using an Elispot assay.

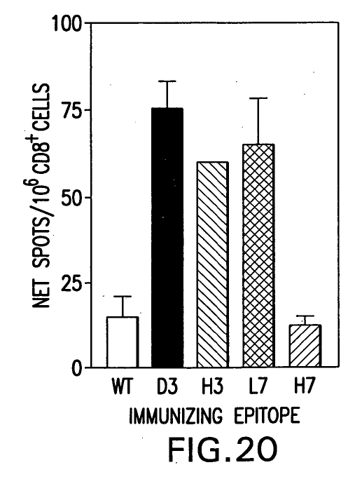

CD8⁺ cells isolated from mice immunized with either WT peptide or the D3, H3, L7, and H7 analogs were analyzed for their ability to release IFNγ when stimulated in the Elispot assay with WT peptide. FIG. 20 shows that while the precursor frequencies of wildtype peptide-reactive CTL were 1/66,000 (15 spots/10⁶) in mice immunized with wildtype peptide, precursor frequencies of wildtype peptide-reactive cells in mice immunized with predicted analogs were approximately 1/15,000 for analogs D3, H3, and L7 (60-75 spots/10⁶ cells), and 1/83,000 (12 spots/10⁶ ) for analog H7. This indicates wildtype-reactive cells were present at a four-fold higher frequency in mice immunized with three out of the four analogs compared to mice immunized with the native peptide. This finding is significant since it implies that in vivo immunization with heteroclitic analogs does indeed induce a higher number of CTL reactive against wildtype peptide, using a more direct assay system where in vitro expansion of in vivo-primed CTL is avoided.

### Example 14

### Synthesis and Analysis of Heteroclitic Analogs Derived from the HLA-A2.1 Supermotif on HLA A2 Superfamily Members

To further validate the heteroclitic substitution rules for other HLA molecules within the A2 superfamily, the panel of nine analogs of the p53.261 epitopes discussed above consisting of three conservative/semiconservative substitutions at positions 3, 5 and 7 are tested for in vivo immunogenicity in transgenic mice expressing one of the following human HLA molecules: A*0202, A*0203, A*0204, A*0205, A*0206, A*0207, A*0209, A*0214, A*6802 and A*6901.

CTLs from the mice immunized with the above-described analogs are tested for induction of at least 100 pg/well of IFNγ production. This IFNγ production typically occurs at much lower peptide concentrations than those induced and restimulated with wildtype peptide (e.g., the p53.261 epitope). These results indicate that our predicted heteroclitic analogs are more potent at inducing higher avidity CTL against the native wildtype epitope than wildtype peptide itself.

Typically, CTLs obtained from animals immunized and restimulated with a wildtype peptide will induce 100 pg/well IFNγ at peptide doses of 5-10μg/ml, whereas CTLs obtained from animals immunized with the above-described analogs, and stimulated and tested in vitro with wildtype peptide, require 10-fold, 100-fold or even 1000-fold lower doses of wildtype peptide respectively, to induce 100 pg/well of IFNγ.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1

| | | |
|---|---|---|
| SEQ ID NO: 1 | IMIGVLVGV | CEA.691 |
| SEQ ID NO: 2 | IMMGVLVGV | CEA.691 M3 |
| SEQ ID NO: 3 | IMIGHLVGV | CEA.691 H5 |
| SEQ ID NO: 4 | KVAELVHFL | MAGE3.112 |
| SEQ ID NO: 5 | KVAEIVHFL | MAGE3.112 I5 |
| SEQ ID NO: 6 | KVAELVWFL | MAGE3.112 W7 |
| SEQ ID NO: 7 | YLQLVFGIEV | MAGE2.157 |
| SEQ ID NO: 8 | YLQLIFGIEV | MAGE2.157 I5 |
| SEQ ID NO: 9 | YLQLFFGIEV | MAGE2.157 F5 |
| SEQ ID NO: 10 | HLFGYSWYK | CEA.61 |
| SEQ ID NO: 11 | HLFPYSWYK | CEA.61 P4 |
| SEQ ID NO: 12 | HLFIYSWYK | CEA.61 I4 |
| SEQ ID NO: 13 | HLFGYSLYK | CEA.61 L7 |
| SEQ ID NO: 14 | HLFGYSMYK | CEA.61 M7 |
| SEQ ID NO: 15 | HLFGYSIYK | CEA.61 I7 |
| SEQ ID NO: 16 | HLFGYSDYK | CEA.61 D7 |
| SEQ ID NO: 17 | HLFGYSGYK | CEA.61 G7 |
| SEQ ID NO: 18 | HLFGYSCYK | CEA.61 C7 |
| SEQ ID NO: 19 | HLFGYSNYK | CEA.61 N7 |
| SEQ ID NO: 20 | EYLQLVFGI | MAGE2.156 |
| SEQ ID NO: 21 | EYIQLVFGI | MAGE2.156 I3 |
| SEQ ID NO: 22 | EYLELVFGI | MAGE2.156 E4 |
| SEQ ID NO: 23 | EYLLLVFGI | MAGE2.156 L4 |
| SEQ ID NO: 24 | EYLQLMFGI | MAGE2.156 M6 |
| SEQ ID NO: 25 | EYLQLLFGI | MAGE2.156 L6 |
| SEQ ID NO: 26 | QYIKANSKFIGITE | Tetanus toxoid |
| SEQ ID NO: 27 | DIEKKIAKMEKASSVFNV | Plasmodium |
| | VNS | falciparum |
| | | circumsporozoite |
| | | protein |
| SEQ ID NO: 28 | GAVDSILGGVATYGAA | Streptococcus 18kD |
| | | protein |
| SEQ ID NO: 29 | AKXVAAWTLKAAa | DR-binding epitope |
| SEQ ID NO: 30 | AKXVAAWTLKAAa | DR-binding epitope |
| SEQ ID NO: 31 | AKXVAAWTLKAAa | DR-binding epitope |
| SEQ ID NO: 32 | APAAAAAAY | |
| SEQ ID NO: 33 | KVFPYALINK | A3 wild-type |
| SEQ ID NO: 34 | TPPAYRPPNAPIL | HBVCore.128 Th |
| SEQ ID NO: 35 | FLPSDFFPSV | HBVCore.18 |
| SEQ ID NO: 36 | APRTLVYLL | HLA-B7 |
| SEQ ID NO: 37 | APETLVYLL | HLA-B7 E3 |
| SEQ ID NO: 38 | APRTWVYLL | HLA-B7 W5 |
| SEQ ID NO: 39 | APRTLVPLL | KLA-B7 P7 |
| SEQ ID NO: 40 | KVHPYALINK | HLA-A3 H3 |
| SEQ ID NO: 41 | KVFPQALINK | HLA-A3 Q5 |
| SEQ ID NO: 42 | KVFPYAKINK | HLA-A3 K7 |
| SEQ ID NO: 43 | VPISHILYIL | MAGE2.170 |
| SEQ ID NO: 44 | VPISHLHIL | MAGE2.170 H7 |
| SEQ ID NO: 45 | VPISHLMIL | MAGE2.170 M7 |
| SEQ ID NO: 46 | VPISHLGIL | MAGE2.170 G7 |
| SEQ ID NO: 47 | VPISHLEIL | MAGE2.170 E7 |
| SEQ ID NO: 48 | VPISHLDIL | MAGE2.170 D7 |
| SEQ ID NO: 49 | SMPPPGTRV | p53.149M2 |
| SEQ ID NO: 50 | CMPPPGTRV | p53.149M2 C1 |
| SEQ ID NO: 51 | SMPPPGPRV | p53.149M2 P7 |
| SEQ ID NO: 52 | GLAPPQHLIRV | p53.Mu.184 |
| SEQ ID NO: 53 | GLTPPQHLIRV | p53.Mu.184 T3 |
| SEQ ID NO: 54 | GLTPPEHLIRV | p53.Mu.184 T3, E6 |
| SEQ ID NO: 55 | GLSRYVARL | HBV Pol.455 |
| SEQ ID NO: 56 | GLSRYVPRL | HBV Pol.455 P7 |
| SEQ ID NO: 57 | ILKEPVHGV | HIV Pol.476 |
| SEQ ID NO: 58 | ILHEPVHGV | HIV Pol.476 H3 |
| SEQ ID NO: 59 | ILLEPVHGV | HIV Pol.476 L3 |
| SEQ ID NO: 60 | LLGRDSFEV | p53.261 |
| SEQ ID NO: 61 | LLDRDSFEV | p53.261 D3 |
| SEQ ID NO: 62 | LLHRDSFEV | p53.261 H3 |
| SEQ ID NO: 63 | LLGRDSLEV | p53.261 L7 |
| SEQ ID NO: 64 | LLGRDSHEV | p53.261 H7 |
| SEQ ID NO: 65 | LLGRNSFEV | p53.261 N5 |
| SEQ ID NO: 66 | LLGRGSFEV | p53.261 G5 |
| SEQ ID NO: 67 | ILIEPVHGV | HIV Pol.476 I3 |

TABLE 2
Compiled rankings and similarity assignments.

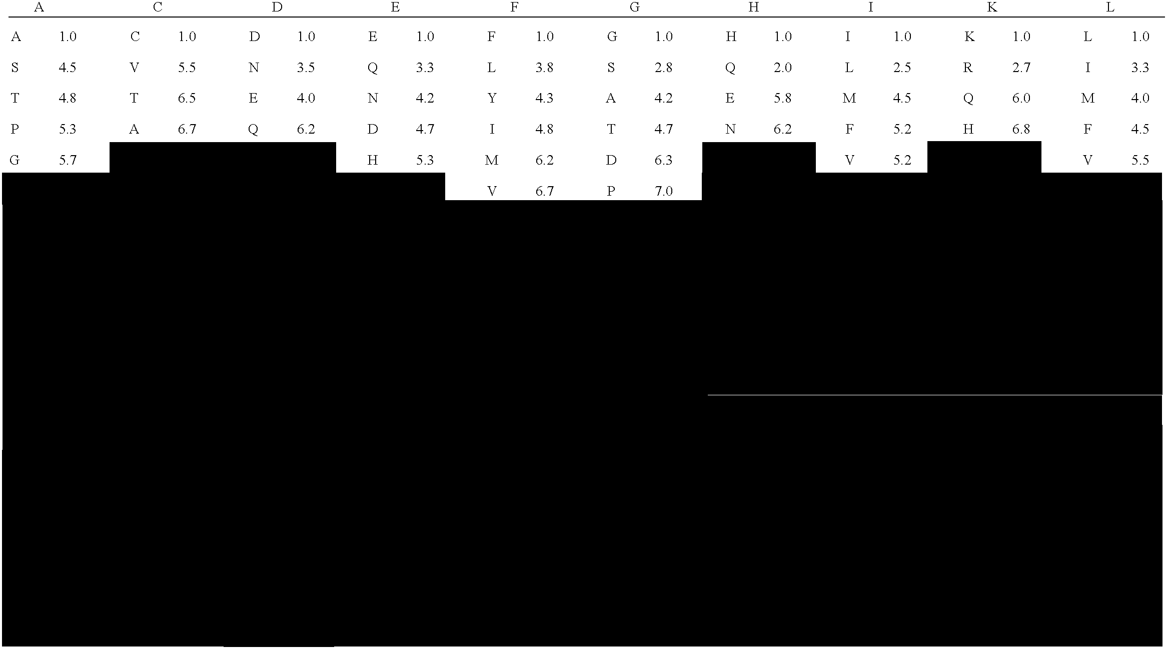

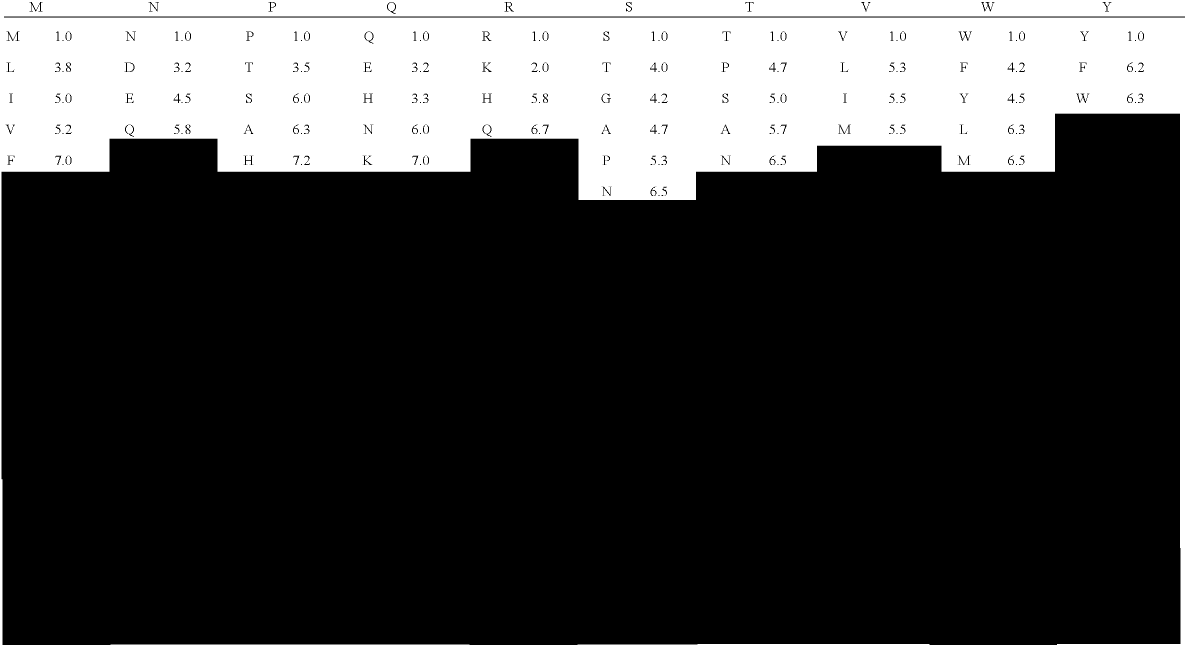

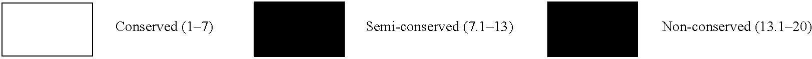

TABLE 3

| | POSITION | POSITION | POSITION |
|---|---|---|---|
| SUPER- | 2 (Primary | 3 (Primary | C Terminus (Primary |
| MOTIFS | Anchor) | Anchor) | Anchor) |
| A1 | T, I, L, V, M, S | | F, W, Y |
| A2 | L, I, Y, M, A, T, Q | | I, V, M, A, T, L |
| A3 | V, S, M, A, T, L, I | | R, K |
| A24 | Y, F, W, I, V, L, M, T | | F, I, Y, W, L, M |
| B7 | P | | V, I, L, F, M, W, Y, A |
| B27 | R, H, K | | F, Y, L, W, M, I, V, A |
| B44 | E, D | | F, W, L, I, M, V, A |
| B58 | A, T, S | | F, W, Y, L, I, V, M, A |
| B62 | Q, L, I, V, M, P | | F, W, Y, M, I, V, L, A |
| MOTIFS | | | |
| A1 | T, S, M | | Y |
| A1 | | D, E, A, S | Y |
| A2.1 | L, M, V, Q, I, A, T | | V, L, I, M, A, T |
| A3 | L, M, V, I, S, A, | | K, Y, R, H, F, A |
| | T, F, C, G, D | | |
| A11 | V, T, M, L, I, S, | | K, R, Y, H |
| | A, G, N, C, D, F | | |
| A24 | Y, F, W, M | | F, L, I, W |
| A*3101 | M, V, T, A, L, I, S | | R, K |
| A*3301 | M, V, A, L, F, I, | | R, K |
| | S, T | | |
| A*6801 | A, V, T, M, S, L, I | | R, K |
| B*0702 | P | | L, M, F, W, Y, A, I, V |
| B*3501 | P | | L, M, F, W, Y, I, V, A |
| B51 | P | | L, I, V, F, W, Y, A, M |
| B*5301 | P | | I, M, F, W, Y, A, I, V |
| B*5401 | P | | A, T, I, V, L, M, F, W, |
| | | | Y |

Bolded residues are preferred, italicized residues are less preferred: A peptide is considered motif-boating If it has primary anchors at each primary anchor position for a motif or supermotif as specified in the above table.

TABLE 4

| | POSITION | POSITION | POSITION |
|---|---|---|---|
| SUPER- | 2 (Primary | 3 (Primary | C Terminus (Primary |
| MOTIFS | Anchor) | Anchor) | Anchor) |
| A1 | T, I, L, V, M, S | | F, W, Y |
| A2 | V, Q, A, T | | I, V, L, M, A, T |
| A3 | V, S, M, A, T, L, I | | R, K |
| A24 | Y, F, W, I, V, L, M, T | | F, I, Y, W, L, M |
| B7 | P | | V, I, L, F, M, W, Y, A |
| B27 | R, H, K | | F, Y, L, W, M, I, V, A |
| B58 | A, T, S | | F, W, Y, L, I, V, M, A |
| B62 | Q, L, I, V, M, P | | F, W, Y, M, I, V, L, A |
| MOTIFS | | | |
| A1 | T, S, M | | Y |
| A1 | | D, E, A, S | Y |
| A2.1 | V, Q, A, T* | | V, L, I, M, A, T |
| A3.2 | L, M, V, I, S, A, | | K, Y, R, H, F, A |
| | T, F, C, G, D | | |
| A11 | V, T, M, L, I, S, | | K, R, H, Y |
| | A, G, N, C, D, F | | |
| A24 | Y, F, W | | F, L, I, W |

*If 2 is V, or Q, the C-term is not L

Bolded residues are preferred, italicized residues are less preferred A peptide is considered motif-bearing if it has primary anchors at each primary anchor position for a motif or supermotif as specified in the above table.

TABLE 5

| | Allelle-specific HLA-supertype members | |
|---|---|---|
| HLA-supertype | Verified^{a} | Predicted^{b} |
| A1 | A*0101, A*2501, A*2601, A*2602, A*3201 | A*0102, A*2604, A*3601, A*4301, A*8001 |
| A2 | A*0201, A*0202, A*0203, A*0204, A*0205, A*0206, A*0207, | A*0208, A*0210, A*0211, A*0212, A*0213 |
| | A*0209, A*0214, A*6802, A*6901 | |
| A3 | A*0301, A*1101, A*3101, A*3301, A*6801 | A*0302, A*1102, A*2603, A*3302, A*3303, A*3401, |
| | | A*3402, A*6601, A*6602, A*7401 |
| A24 | A*2301, A*2402, A*3001 | A*2403, A*2404, A*3002, A*3003 |
| B7 | B*0702, B*0703, B*0704, B*0705, B*1508, B*3501, B*3502, B*3503, | B*1511, B*4201, B*5901 |
| | B*3503, B*3504, B*3505, B*3506, B*3507, B*3508, B*5101, B*5102, | |
| | B*5103, B*5104, B*5105, B*5301, B*5401, B*5501, B*5502, B*5601, | |
| | B*5602, B*6701, B*7801 | |
| B27 | B*1401, B*1402, B*1509, B*2702, B*2703, B*2704, B*2705, B*2706, | B*2701, B*2707, B*2708, B*3802, B*3903, B*3904, |
| | B*3801, B*3901, B*3902, B*7301 | B*3905, B*4801, B*4802, B*1510, B*1518, B*1503 |
| B44 | B*1801, B*1802, B*3701, B*4402, B*4403, B*4404, B*4001, B*4002, | B*4101, B*4501, B*4701, B*4901, B*5001 |
| | B*4006 | |
| B58 | B*5701, B*5702, B*5801, B*5802, B*1516, B*1517 | |
| B62 | B*1501, B*1502, B*1513, B*5201 | B*1301, B*1302, B*1504, B*1505, B*1506, B*1507, |
| | | B*1515, B*1520, B*1521, B*1512, B*1514, B*1510 |

^{a}Verified alleles include alleles whose specificity has been determined by pool sequencing analysis, peptide binding assays, or by analysis of the sequences of CTL epitopes.
^{b}Predicted alleles are alleles whose specificity is predicted on the basis of B and F pocket structure to overlap with the supertype specificity.

TABLE 6

Summary of heteroclitic analogs of CEA.61 and
MAGE2.156

| | | | | | | |
|---|---|---|---|---|---|---|
| | | | SEQ ID | Substi- | | |
| Epitope | HLA | Analogs | NO: | tution | Pos. | Type |

| | | | | | | | |
|---|---|---|---|---|---|---|---|
| CEA.61 | A3 | HLFPYSWYK | SEQ ID | G | P | 4 | Consv |
| | | | NO: 11 | | | | |
| | | HLFIYSWYK | SEQ ID | G | I | 4 | Non- |
| | | | NO: 12 | | | | Consv |
| | | HLFGYSLYK | SEQ ID | W | L | 7 | Consv |
| | | | NO: 13 | | | | |
| | | HLFGYSMYK | SEQ ID | W | M | 7 | Consv |
| | | | NO: 14 | | | | |
| | | HLFGYSIYK | SEQ ID | W | I | 7 | Semi- |
| | | | NO: 15 | | | | Consv |
| | | HLFGYSDYK | SEQ ID | W | D | 7 | Non- |
| | | | NO: 16 | | | | Consv |
| | | HLFGYSGYK | SEQ ID | W | G | 7 | Non- |
| | | | NO: 17 | | | | Consv |
| | | HLFGYSCYK | SEQ ID | W | C | 7 | Non- |
| | | | NO: 18 | | | | Consv |
| | | HLFGYSNYK | SEQ ID | W | N | 7 | Non- |
| | | | NO: 19 | | | | Consv |
| MAGE2.156 | A24 | EYIQLVFGI | SEQ ID | L | I | 3 | Consv |
| | | | NO: 21 | | | | |
| | | EYLELVFGI | SEQ ID | Q | E | 4 | Consv |
| | | | NO: 22 | | | | |
| | | EYLLLVFGI | SEQ ID | Q | L | 4 | Non- |
| | | | NO: 23 | | | | Consv |
| | | EYLQLMFGI | SEQ ID | V | M | 6 | Consv |
| | | | NO: 24 | | | | |
| | | EYLQLLFGI | SEQ ID | V | L | 6 | Consv |
| | | | NO: 25 | | | | |

TABLE 7

Characterization of heteroclitic analogs identified from tumor and viral antigens.

| | | | | | | | | |
|---|---|---|---|---|---|---|---|---|
| | Seq | | | | | | | A*0201 |
| | ID | | Heteroclitic | Type of | Position of | Th1 | Th2 | binding |
| Antigen | NO: | Sequence | substitution | substitution | substitution | cytokines^{a} | cytokines^{b} | (IC50, nM)^{d} |

| | | | | | | | | |
|---|---|---|---|---|---|---|---|---|
| CEA.691 | 1 | IMIGVLVGV | None (WT) | None | | 1 | 10 | 54 |
| CEA.691 M3 | 2 | IMMGVLVGV | I→M | Conservative | 3 | 10⁻⁵ | 1 | 27 |
| CEA.691 H5 | 3 | IMIGHLVGV | V→H | Semi- | 5 | 10⁻⁷ | 10⁻¹ | 16 |
| | | | | conservative | | | | |
| MAGE3.112 | 4 | KVAELVHFL | None (WT) | None | | 1 | NS^{c} | 94 |
| MAGE3.112 I5 | 5 | KVAEIVHFL | L→I | Conservative | 5 | 10⁻⁴ | NS | 66 |
| MAGE3.112 W7 | 6 | KVAELVWFL | H→W | Semi- | 7 | 10⁻⁷ | NS | 7 |
| | | | | conservative | | | | |
| MAG32.157 | 7 | YLQLVFGIEV | None (WT) | None | | 1 | 10 | 40 |
| MAGE2.157 I5 | 8 | YLQLIFGIEV | V→I | Conservative | 5 | 10⁻⁴ | 10⁻² | 476 |
| MAGE2.157 F5 | 9 | YLQLFFGIEV | V→F | Semi- | 5 | 10⁻² | 10⁻² | 212 |
| | | | | conservative | | | | |

^{a}Minimum peptide concentration (μg/ml) required to induce 100 pg/well of IFNγ (Th1 cytokines)
^{b}Minimum peptide concentration (μg/ml) required to induce 50 pg/ml of IL10 or IL5 (Th2 cytokines)
^{c}NS, cytokine levels not significant (<5 pg/ml)
^{d}A relative binding change of four-fold or more compared to wild-type peptide is considered significant and is indicated in bold

TABLE 8
Summary of heteroclitic analogs of MAGE2.170

| | Heteroclitic | Type of | Position of | B*0702 binding |
|---|---|---|---|---|
| Antigen | substitution | substitution | Substitution | (IC50, nM) |
| MAGE2.170 | None (WT) | None | | 112 |
| MAGE2.170H7 | Y → H | Semi-conservative | 7 | 75 |
| MAGE2.170M7 | Y → M | Semi-conservative | 7 | 69 |
| MAGE2.170G7 | Y → G | Non-conservative | 7 | 105 |
| MAGE2.170E7 | Y → E | Non-conservative | 7 | 186 |
| MAGE2.170D7 | Y → D | Non-conservative | 7 | 1276 |

TABLE 9

| | |
|---|---|
| CEA | |
| MESPSAPPHRWCIPWQRLLLTASLLTFWNPPTTAKLTIESTPFNVAEGKEVLLLVHNLPQHLF | (SEQ ID NO: 68) |
| GYSWYKGERVDGNRQIIGYVIGTQQATPGPAYSGREIIYPNASLLIQNIIQNDTGFYTLHVIKSDLVNE | |
| EATGQFRVYPELPKPSISSNNSKPVEDKDAVAFTCEPETQDATYLWWVNNQSLPVSPRLQLSNGNRTLT | |
| LFNVTRNDTASYKCETQNPVSARRSDSVILNVLYGPDAPTISPLNTSYRSGENLNLSCHAASNPPAQYS | |
| WFVNGTFQQSTQELFIPNITVNNSGSYTCQAHNSDTGLNRTTVTTITVYAEPPKPFITSNNSNPVEDED | |
| AVALTCEPEIQNTTYLWWVNNQSLPVSPRLQLSNDNRTLTLLSVTRNDVGPYECGIQNELSVDHSDPVI | |
| LNVLYGPDDPTISPSYTYYRPGVNLSLSCHAASNPPAQYSWLIDGNIQQHTQELFISNITEKNSGLYTC | |
| QANNSASGHSRTTVKTITVSAELPKPSISSNNSKPVEDKDAVAFTCEPEAQNTTYLWWVNGQSLPVSPR | |
| LQLSNGNRTLTLFNVTRNDARAYVCGIQNSVSANRSDPVTLDVLYGPDTPIISPPDSSYLSGANLNLSC | |
| HSASNPSPQYSWRINGIPQQHTQVLFIAKITPNNNGTYACFVSNLATGRNNSIVKSITVSASGTSPGLS | |
| AGATVGIMIGVLVGVALI | |
| MAGE2 | |
| MPLEQRSQHCKPEEGLEARGEALGLVGAQAPATEEQQTASSSSTLVEVTLGEVPAADSPSPPH | (SEQ ID NO: 69) |
| SPQGASSFSTTINYTLWRQSDEGSSNQEEEGPRMFPDLESEFQAAISRKMVELVHFLLLKYRAREPVTK | |
| AEMLESVLRNCQDFFPVIFSKASEYLQLVFGIEVVEVVPISHLYILVTCLGLSYDGLLGDNQVMPKTGL | |
| LIIVLAIIAIEGDCAPEEKIWEELSMLEVFEGREDSVFAHPRKLLMQDLVQENYLEYRQVPGSDPACYE | |
| FLWGPRALIETSYVKVLHHTLKIGGEPHISYPPLHERALREGEE | |

TABLE 10

| | |
|---|---|
| Human Serum Albumin | |
| ACCESSION CAA23754 | |
| mkwvtfisllflfssaysrgvfrrdahksevahrfkdlgeenfkalvliafaqylqqcpfedhvklvne | (SEQ ID NO: 70) |
| vtefaktcvadesaencdkslhtlfgdklctvatlretygemadccakqepernecflqhkddnpnlpr | |
| lvrpevdvmctafhdneetflkkylyeiarrhpyfyapellffakrykaafteccqaadkaacllpkld | |
| elrdegkassakqrlkcaslqkfgerafkawavarlsqrfpkaefaevsklvtdltkvhtecchgdlle | |
| caddradlakyicenqdsissklkeccekpllekshciaevendempadlpslaadfveskdvcknyae | |
| akdvflgmflyeyarrhpdysvvlllrlaktyettlekccaaadphecyakvfdefkplveepqnlikq | |
| ncelfkqlgeykfqnallvrytkkvpqvstptlvevsrnlgkvgskcckhpeakrmpcaedylsvvlnq | |
| lcvlhektpvsdrvtkccteslvnrrpcfsalevdetyvpkefnaetftfhadictlsekerqikkqta | |
| lvelvkhkpkatkeqlkavmddfaafvekcckaddketcfaeegkklvaasqaalgl | |
| HBV core | |
| MQLFHLCLIISCSCPTVQASKLCLGWLWGMDIDPYKEFGATVELLSFLPSDFFP | (SEQ ID NO: 71) |
| SVRDLLDTASALYREALESPEHCSPHHTALRQAILCWGELMTLATWVGVNLED | |
| PASRDLVVSYVNTNMGLKFRQLLWFHISCLTFGRETVIEYLVSFGVWIRTPPAY | |
| RPPNAPILSTLPETTVVRRRGRSPRRRTPSPRRRRSQSPRRRRSQSRESQC | |

TABLE 11

| | |
|---|---|
| Thyroglobulin | |
| ACCESSION NP_003226 | |
| malvleiftllasicwvsanifeyqvdaqplrpcelqretaflkqadyvpqcaedgsfqtvqcqndgrs | (SEQ ID NO: 72) |
| cwcvgangsevlgsrqpgrpvaclsfcqlqkqqillsgyinstdtsylpqcqdsgdyapvqcdvqqvqc | |
| wcvdaegmevygtrqlgrpkrcprsceirnrrllhgvgdksppqcsaegefmpvqckfvnttdmmifdl | |
| vhsynrfpdafvtfssfqrrfpevsgychcadsqgrelaetglellldeiydtifagldlpstftettl | |
| yrilqrrflavqsvisgrfrcptkceverftatsfghpyvpscrrngdyqavqcqtegpcwcvdaqgke | |
| mhgtrqqgeppscaegqscaserqqalsrlyfgtsgyfsqhdlfsspekrwasprvarfatscpptike | |
| lfvdsgllrpmvegqsqqfsvsenllkeairaifpsrglarlalqfttnpkrlqqnlfggkflvnvgqf | |
| nlsgalgtrgtfnfsqffqqlglasflnggrqedlakplsvgldsnsstgtpeaakkdgtmnkptvgsf | |
| gfeinlqenqnalkflasllelpefllflghaisvpedvardlgdvmetvlssqtceqtperlfvpsct | |
| tegsyedvqcfsgecwcvnswgkelpgsrvrggqprcptdcekqrarmqslmgsqpagstlfvpactse | |
| ghflpvqcfnsecycvdaegqaipgtrsaigkpkkcptpcqlqxeqaflrtvqallsnssmlptlsdty | |
| ipqcstdgqwrqvqcngppeqvfelyqrweaqnkgqdltpakllvkimsyreaasgnfslfiqslyeag | |
| qqdvfpvlsqypslqdvplaalegkrpqprenillepylfwqilngqlsqypgsysdfstplahfdlrn | |
| cwcvdeagqelegmrsepsklptcpgsceeaklrvlqfireteeivsasnssrfplgesflvakgirlr | |
| nedlglpplfppreafaeqflrgsdyairlaaqstlsfyqrrrfspddsagasallrsgpyxpqcdafg | |
| swepvqchagtghcwcvdekggfipgsltarslqipqcpttceksrtsgllsswkqarsqenpspkdlf | |
| vpacletgeyarlqasgagtwcvdpasgeelrpgssssaqcpslcnvlkagvlsrrvspgyvpacraed | |
| ggfspvqcdqaqgscwcvmdsgeevpgtrvtggqpacesprcplpfnasevvggtilcetisgptgsam | |
| qqcqllcrqgswsvfppgplicslesgrwesqlpqpracqrpqlwqtiqtqghfqlqlppgkmcsadya | |
| gllqtfqvfildeltargfcqiqvktfgtlvsipvcnnssvqvgcltrerlgvnvtwksrledipvasl | |
| pdlhdieralvgkdllgrftdliqsgsfqlhldsktfpaetirflqgdhfgtsprtwfgcsegfyqvlt | |
| seasqdglgcvkcpegsysqdeecipcpvgfyqeqagslac | |
| vpcpvgrttisagafsqthcvtdcqrneaglqcdqngqyrasqkdrgsgkafcvdgegrrlpwweteap | |
| ledsqclmmqkfekvpeskvifdanapvavrskvpdsefpvmqcltdctedeacsfftvsttepeiscd | |
| fyawtsdnvacmtsdqkrdalgnskatsfgslrcqvkvrshgqdspavylkkgqgstttlqkrfeptgf | |
| gnmlsglynpivfsasganltdahlfcllacdrdlccdgfvltqvqggaiicgllsspsvllcnvkdwm | |
| dpseawanatcpgvtydqeshqvilrlgdqefiksltplegtqdtftnfqqvylwkdsdmgsrpesmgc | |
| rkbtvprpaspteaglttelfspvdlnqvivngnqslssqkhwlfkhlfsaqqanlwclsrcvqehsfc | |
| qlaeitesaslyftctlypeaqvcddimesnaqgcrlilpqmpkalfrkkviledkvknfytrlpfqkl | |
| mgisirnkvpmseksisngffecerrcdadpcctgfgflnvsqlkggevtcltlnslgiqmcseengga | |
| wrildcgspdievhtypfgwyqkpiaqnnapsfcplvvlpsltekvsldswqslalssvvvdpsirhfd | |
| vahvstaatsnfsavrdlclsecsqheaclittlqtqpgavrcmfyadtqscthslqgqncrlllreea | |
| thiyrkpgisllsyeasvpsvpisthgrllgrsqaiqvgtswkqvdqflgvpyaapplaerrfqapepl | |
| nwtgswdaskprascwqpgtrtstspgvsedclylnvfipqnvapnasvlvffhntmdreesegwpaid | |
| gsflaavgnlivvtasyrvgvfgflssgsgevsgnwglldqvaaltwvqthirgfggdprrvslaadrg | |
| gadvasihlltaratnsqlfrravlmggsalspaavisheraqqqaialakevscpmsssqevvsclrq | |
| kpanvlndaqtkllavsgpfhywgpvidghflrepparalkrslxvevdlligssqddglinrakavkq | |
| feesqgrtssktafyqalqnslggedsdarveaaatwyyslehstddyasfsralenatrdyfiicpii | |
| dmasawakrargnvfmyhapenyghgslelladvqfalglpfypayegqfsleekslslkimqyfshfi | |
| rsgnpnypyefsrkvptfatpwpdfvpraggenykefsellpnrqglkkadcsfwskyisslktsadga | |
| kggqsaeseeeeltagsglredllslqepgsktysk | |

TABLE 12
Tumor Associated Antigens and Genes (TAA)

| ANTIGEN | REFERENCE |
|---|---|
| MAGE 1 | (Traversari C., Boon T, J.Ex. Med 176: 1453, 1992) |
| MAGE 2 | (De Smet C., Boon T, Immunogenetics, 39(2)121-9, |
| | 1994) |
| MAGE 3 | (Gaugler B., Boon T, J.Ex. Med 179: 921, 1994) |
| MAGE-11 | (Jurk M., Winnacker L, Int.J.Cancer 75, 762-766, |
| | 1998) |
| MAGE-A10 | (Huang L., Van Pel A, J.Immunology, 162: |
| | 6849-6854) |
| BAGE | (Boel P., Bruggen V, Immunity 2: 167, 1995) |
| GAGE | (Eynde V., Boon T, J.Exp. Med 182: 689, 1995) |
| RAGE | (Gaugler B., Eynde V, Immunogenetics, 44: 325, |
| | 1996) |
| MAGE-C1 | (Lucas S., Boon T, Cancer Research, 58, |
| | 743-752, 1998) |
| LAGE-1 | (Lethel B., Boon T, Int J cancer, 10; |
| | 76(6) 903-908 |
| CAG-3 | (Wang R-Rosenberg S, J.Immunology, 161: |
| | 3591-3596, 1998) |
| DAM | (Fleischhauer K., Traversari C, Cancer Research, |
| | 58, 14, 2969, 1998) |
| MUC1 | (Karanikas V., McKenzie IF, J.clnical investigation, |
| | 100: 11, 1-10, 1997) |
| MUC2 | (Bohm C., Hanski, Int.J.Cancer 75, 688-693, 1998) |
| MUC18 | (Putz E., Pantel K, Cancer Res 59(1): 241-248, |
| | 1999) |
| NY-ES0-1 | (Chen Y., Old LJ PNAS, 94, 1914-18, 1997) |
| MUM-1 | (Coulie P., Boon T, PNAS 92: 7976, 1995) |
| CDK4 | (Wolfel T., Beach D, Science 269: 1281, 1995) |
| BRCA2 | (Wooster R-Stratton M, Nature, 378, 789-791, |
| | 1995) |
| NY-LU-1 | (Gure A., Chen, Cancer Research, 58, 1034-41, |
| | 1998) |
| NY-LU-7 | (Gure A., Chen, Cancer Research, 58, 1034-41, |
| | 1998) |
| NY-LU-12 | (Gure A., Chen, Cancer Research, 58, 1034-41, |
| | 1998) |
| CASP8 | (Mandruzzato S., Bruggen P, J.Ex.Med 186, 5, |
| | 785-793, 1997) |
| RAS | (Sidransky D., Vogelstein B, Science, 256: 102) |
| KIAA0205 | (Gueguen M., Eynde, J.Immunology, 160: |
| | 6188-94, 1998) |
| SCCs | (Molina R., Ballesta AM, Tumor Biol, 17(2): |
| | 81-9, 1996) |
| p53 | (Hollstein M., Harris CC, Science, 253, |
| | 49-53, 1991) |
| p73 | (Kaghad M., Caput D, Cell; 90(4): 809-19, |
| | 1997) |
| CEA | (Muraro R., Schlom J, Cancer Research, 45: |
| | 5769-55780, 1985) |
| Her 2/neu | (Disis M., Cheever M, Cancer Res 54: 1071, 1994) |
| Melan-A | (Coulie P., Boon T, J.Ex.Med; 180: 35, 1994) |
| gp100 | (Bakker A., Figdor, J.Ex.Med 179: 1005, 1994) |
| Tyrosinase | (Wolfel T., Boon T, E.J.I 24: 759, 1994) |
| TRP2 | (Wang R., Rosenberg S.A, J.Ex.Med 184: 2207, |
| | 1996) |
| gp75/TRP1 | (Wang R., Rosenberg S.A, J.Ex,Med 183: 1131, |
| | 1996) |
| PSM | (Pinto J.T., Heston W.D.W., Clin Cancer Res 2(9); |
| | 1445-1451, 1996) |
| PSA | (Correale P., Tsang K, J. Nati cancer institute, 89: |
| | 293-300, 1997) |
| PT1-1 | (Sun Y., Fisher PB, Cancer Research, 57(1): |
| | 18-23, 1997) |
| B-catenin | (Robbins P., Rosenberg SA, J.Ex.Med 183: |
| | 1185, 1996) |
| PRAME | (Neumann E., Seliger B, Cancer Research, 58, |
| | 4090-4095, 1998) |
| Telomearse | (Kishimoto K., Okamoto E, J Surg Oncol, 69(3): |
| | 119-124, 1998) |
| FAK | (Kornberg LJ, Head Neck, 20(8): 745-52, 1998) |
| Tn antigen | (Wang Bl, J Submicrosc Cytol Path, 30(4): |
| | 503-509, 1998) |
| cyclin D1 | (Linggui K., Yaowu Z, Cancer Lett 130(1-2), |
| protein | 93-101, 1998) |
| NOEY2 | (Yu Y., Bat RC, PNAS, 96(1): 214-219, 1999) |
| EGF-R | (Biesterfeld S.- Cancer Weekly, Feb. 15, 1999) |
| SART-1 | (Matsumoto H., Itoh K, Japanese Journal of Cancer |
| | Research, 59, iss12, 1292-1295, 1998) |
| CAPB | (Cancer Weekly, March 29,4-5, 1999) |
| HPVE7 | (Rosenberg S.A.Immunity, 10, 282-287, 1999) |
| p15 | (Rosenberg S.A., Immunity, 10, 282-287, 1999) |
| Folate | (Gruner B.A., Weitman S.D., Investigational New Drugs, |
| receptor | Vol16, iss3, 205-219, 1998) |
| CDC27 | (Wang R.F., Rosenberg SA, Science, vol 284, |
| | 1351-1354, 1999) |
| PAGE-1 | (Chen, J. Biol. Chem: 273: 17618-17625, 1998) |
| PAGE-4 | (Brinkimann: PNAS, 95: 10757, 1998) |
| Kallikrein 2 | (Darson: Urology, 49: 857-862, 1997) |
| PSCA | (Relter R., PNAS, 95: 1735-1740, 1998) |
| DD3 | (Bussemakers M.J.G, European Urology, 35: 408-412, |
| | 1999) |
| RBP-1 | (Takahashi T., British Journal of Cancer, 81(2): |
| | 342-349, 1999) |
| RU2 | (Eybde V.D., J.Exp.Med, 190 (12): 1793-1799, 1999) |
| Folate | (Kim D., Anticancer Research, 19: 2907-2916, 1999) |
| binding | |
| protein | |
| EGP-2 | (Heidenreich R., Human Gene Therapy, 11:9-19, 2000) |

TABLE 13

HLA-A3 Candidate Epitopes for CEA, HER2, p53, and MAGE2/3

| | | | | | | | | | |
|---|---|---|---|---|---|---|---|---|---|
| SEQ ID | Epimmune | | | | A*0301 | A*1101 | A*3101 | A*3301 | A*6801 |
| NO: | ID | Peptide | Sequence | Source | nM | nM | nM | nM | nM |

| | | | | | | | | | |
|---|---|---|---|---|---|---|---|---|---|
| 73 | 1355.02 | 34.0212 | TISPLNTSYK | CEA.241K10 | 61 | 182 | — | — | 116 |
| 74 | 1264.04 | 18.0283 | RTLTLLSVTR | CEA.376 | 524 | 55 | 6.0 | 1036 | 160 |
| 75 | 1371.07 | 1371.07 | IVPSYTYYR | CEA.420V2 | 92 | 13 | 26 | 58 | 2.6 |
| 76 | 1264.01 | 18.0282 | RTLTLFNVTR | CEA.554 | 111 | 13 | 5.0 | 1611 | 99 |
| 77 | 1264.05 | 18.0113 | HTQVLFIAK | CEA.636 | 1183 | 35 | 106 | 132 | 160 |
| 78 | 1355.04 | 34.0148 | FVSNLATGR | CEA.656 | 5790 | 122 | 333 | 104 | 8.2 |
| 79 | | | SSFTTINK | MAGE2.69K9 | 69 | 3.0 | 2195 | — | 26 |
| 80 | 1093.10 | 1093.10 | TTINYTLWR | MAGE2.73 | 204 | 11 | 237 | 171 | 17 |
| 81 | 1356.11 | 34.0156 | SMLEVFEGK | MAGE2.226K9 | 116 | 3.8 | 120 | 387 | 2581 |
| 82 | 1355.12 | 34.0031 | SVFAHPRK | MAGE2.237 | 78 | 74 | 1385 | — | 182 |
| 83 | 1371.74 | 1371.74 | IVYPPLHER | MAGE2.299V2 | 117 | 375 | 95 | 32 | 14 |
| 84 | 1371.69 | 1371.69 | YVFPVIFSK | MAGE3.138V2 | 24 | 3.0 | 2769 | 784 | 1.7 |
| 85 | 1355.15 | 34.0162 | SVLEVFEGK | MAGE3.226K9 | 83 | 6.7 | 129 | 460 | 186 |
| 86 | 1355.14 | | LVHFLLLKK | MAGE2/3.116K9 | 21 | 4.3 | — | — | 381 |
| 87 | 1095.51 | | VVFGILIKR | Her2/neu.669 | 100 | 8.3 | 13 | 78 | 4.0 |
| 88 | 1095.23 | | KIRKYTMRR | Her2/neu.681 | 15 | 3333 | 16 | 4028 | — |
| 89 | 1095.47 | | VLRENTSPK | Her2/neu.754 | 28 | 462 | 129 | 290 | — |
| 90 | 1095.31 | | LVKSPNHVK | Her2/neu.852 | 23 | 86 | 182 | 784 | 73 |
| 91 | | | KVTDFGLAR | Her2/neu.860V2 | 201 | 76 | 106 | — | 133 |
| 92 | 1355.06 | | MALESILRR | Her2/neu.889 | 3235 | 253 | 191 | 132 | 127 |
| 93 | 1095.32 | | LVSEFSRMAR | Her2/neu.972 | 1528 | 182 | 49 | 126 | 36 |
| 94 | 1355.07 | | ASPLDSTFYR | Her2/neu.997 | — | 90 | 150 | 2071 | 154 |
| 95 | 1355.17 | | KTYQGSYGFK | p53.101K10 | 22 | 14 | 129 | — | 67 |
| 96 | 1096.01 | | CTYSPALNK | p53.124 | 24 | 5.5 | 1500 | 518 | 36 |
| 97 | 1355.19 | | GTRVRAMAIYK | p53.154 | 10 | 18 | 16 | — | 533 |
| 98 | | | RVRAMAIYR | p53.156R9 | 41 | 1667 | 9 | 138 | 667 |
| 99 | 1355.26 | | RVCACPGR | p53.273 | 31 | 122 | 106 | 193 | 571 |

TABLE 14

HLA-A24 Candidate Epitopes for CEA, HER2, p53,
and MAGE2/3

| | | | | | |
|---|---|---|---|---|---|
| | | | | A*2402 | SEQ ID |
| Peptide | AA | Sequence | Source | nM | NO: |

| | | | | | |
|---|---|---|---|---|---|
| 15.0006 | 9 | IYPNASLLI | CEA.101 | 1.7 | 100 |
| 15.0008 | 9 | LYGPDAPTI | CEA.234 | 57 | 101 |
| 15.0192 | 10 | QYSWFVNGTF | CEA.268 | 3.5 | 102 |
| 15.0010 | 9 | VYAEPPKPF | CEA.318 | 41 | 103 |
| 52.0144 | 11 | TYLWWVNNQSL | CEA.353 | 46 | 104 |
| 57.0077 | 10 | YYRPGVNLSF | CEA.426F10 | 10 | 105 |
| 57.0078 | 10 | QYSWLIDGNF | CEA.446F10 | 60 | 106 |
| 52.0147 | 11 | TYLWWVNGQSL | CEA.531 | 92 | 107 |
| 15.0015 | 9 | LYGPDTPII | CEA.590 | 46 | 108 |
| 57.0079 | 10 | SYLSGANLNF | CEA.604F10 | 10 | 109 |
| 15.0017 | 9 | TYACFVSNL | CEA.652 | 10 | 110 |
| 57.0060 | 9 | MYPDLESEF | MAGE2.97Y2 | 52 | 111 |
| 57.0085 | 10 | LYILVTCLGF | MAGE2.175F10 | 18 | 112 |
| 10.0018 | 9 | VMPKTGLLI | MAGE2.195 | 52 | 113 |
| 52.0072 | 8 | LWGPRALI | MAGE2.272 | 100 | 114 |
| 57.0088 | 10 | SYVKVLHHTF | MAGE2.282F10 | 34 | 115 |
| 10.0082 | 9 | NWQYFFPVI | MAGE3.142 | 23 | 116 |
| 57.0092 | 10 | LYIFATCLGF | MAGE3.175F10 | 10 | 117 |
| 10.0033 | 9 | IMPKAGLLI | MAGE3.195 | 29 | 118 |
| 52.0102 | 10 | SYPPLHEWVL | MAGE3.300 | 20 | 119 |
| 57.0052 | 9 | PYVSRLLGF | Her2/neu.780F9 | 9.2 | 120 |
| 57.0056 | 9 | SYGVTVWEF | Her2/neu.907F9 | 26 | 121 |
| 52.0163 | 11 | VYMIMVKCWMI | Her2/neu.951 | 6.7 | 122 |
| 57.0058 | 9 | RYRELVSEF | Her2/neu.968Y2 | 36 | 123 |
| 52.0103 | 10 | TYQGSYGFRL | p53.102 | 100 | 124 |
| 57.0096 | 10 | TYQGSYGFRF | p53.102F10 | 30 | 125 |
| 52.0104 | 10 | TYSPALNKMF | p53.125 | 2.4 | 126 |

TABLE 15

TAA-derived A1 candidates

| | | | | | |
|---|---|---|---|---|---|
| | | | SEQ | | |
| | | | ID | | A*0101 |
| Peptide | AA | Sequence | NO: | Source | nM |

| | | | | | |
|---|---|---|---|---|---|
| 52.0109 | 11 | RSDSVILNVLY | 127 | CEA.225 | 47 |
| 57.0008 | 9 | ITDNNSGSY | 128 | CEA.289.D3 | 96 |
| 52.0113 | 11 | HSDPVILNVLY | 129 | CEA.403 | 26 |
| 57.0104 | 10 | PTDSPSYTYY | 130 | CEA.418.D3 | 1.1 |
| 57.0013 | 9 | AADNPPAQY | 131 | CEA.439.D3 | 45 |
| 57.0014 | 9 | ITDKNSGLY | 132 | CEA.467.D3 | 12 |
| 52.0116 | 11 | RSDPVTLDVLY | 133 | CEA.581 | 7.8 |
| 18.0186 | 10 | HSASNPSPQY | 134 | CEA.616 | 74 |
| 57.0022 | 9 | VMDGVGSPY | 135 | Her2/neu.773.D3 | 40 |
| 57.0113 | 10 | CTQIAKGMSY | 136 | Her2/neu.826.T2 | 19 |
| 1.0346 | 9 | LLDIDETEY | 137 | Her2/neu.869 | 3.3 |
| 1.0749 | 10 | FTHQSDVWSY | 138 | Her2/neu.899 | 9.3 |
| 57.0115 | 10 | PADPLDSTFY | 139 | Her2/neu.996.D3 | 19 |
| 57.0116 | 10 | MTDLVDAEEY | 140 | Her2/neu.1014.T2 | 2.3 |
| 57.0025 | 9 | LTDSPQPEY | 141 | Her2/neu.1131.D3 | 32 |
| 13.0090 | 10 | FSPAFDNLYY | 142 | Her2/neu.1213 | 4.5 |
| 57.0118 | 10 | GTDTAENPEY | 143 | Her2/neu.1239.D3 | 26 |
| 57.0120 | 10 | ASDFSTTINY | 144 | MAGE2.68.D3 | 25 |
| 57.0003 | 8 | VTDLGLSY | 145 | MAGE2.179.D3 | 2.7 |
| 15.0112 | 9 | MQDLVQENY | 146 | MAGE2.247 | 17 |
| 1044.01 | 10 | ASSLPTTMNY | 147 | MAGE3.68 | 11 |
| 57.0032 | 9 | GTVVGNWQY | 148 | MAGE3.137.TZ | 36 |
| 1044.07 | 9 | EVDPIGHLY | 149 | MAGE3.168 | 6.8 |
| 57.0124 | 10 | LTDHFVQENY | 150 | MAGE3.246.D3 | 2.3 |
| 57.0034 | 9 | ITGGPHISY | 151 | MAGE3.293.T2 | 36 |
| 57.0125 | 10 | PTQKTYQGSY | 152 | p53.98.T2 | 36 |
| 57.0126 | 10 | GTDKSVTCTY | 153 | p53.117.D3 | 42 |
| 57.0127 | 10 | RVDGNLRVEY | 154 | p53.196.D3 | 46 |
| 52.0136 | 11 | GSDCTTIHYNY | 155 | p53.226 | 68 |

TABLE 16

Primary amino acid sequence
Polypeptide 1 (SEQ ID NO: 156)
MGMQVQIQSLFLLLLWVPGSRGVPISHLDILKKLSEYLQLVGAAAISPSYTYYRKAAATYACFVSNLKVTDFG
AAAHLFGYSIYKNAQYSWFVNGTFKAAAKVFGSLAFVNAAAPYVSRLLGINIMIGHLVGVNLLTFWNPPVIVYI
ERNAAAEYLQLMFGINAIMPKAGLLINKTYQGSYGFKKAAARVRAMAIYRNAAARYARDPQRFGAAAKLCPV
VNASMPPPGTRVGAAAVVLGVVFGIAKFVAAWTLKAAAKVAEIVHFLNTYSPALNKMFKAASYGFRLGFFKA
PSTTINKKAAAVVFGILIKR
Polypeptide 2 (SEQ ID NO: 157)
MGMQVQIQSLFLLLLWVPGSRGIVYPPLHERNAAAEYLQLLFGINAIMPKAGLLINKTYQGSYGFKKAAARVF
YRNAAARYARDPQRFGAAAKLCPVQLWVNASMPPPGTRVGAAAVVLGVVFGIAKFVAAWTLKAAAKVAEL
NAAATYSPALNKMFKAASYGFRLGFFKAAASSFSTTINKKAAAVVFGILIKRVPISHLGILKKLSEYLQLVGAAA
YTYYRKAAATYACFVSNLKVFGSLAFVNAAAPYVSRLLGINAHLFGYSDYKNAQYSWFVNGTFKAAAKVTDF
RNIMMGHLVGVNLLTFWNPPV
Polypeptide 3 (SEQ ID NO: 158)
MGMQVQIQSLFLLLLWVPGSRGTYSPALNKMFKAASYGFRLGFFKAAASSFSTTINKKAAAVVFGILIKRNAA
VAAWTLKAAAKVAEIVHFLKVTDFGLARGAAAHLFPYSWYKNATYACFVSNLKAAAVPISHLEILKKLSEYLQI
AAISPSYTYYRKAAAQYSWFVNGTFKAAAKVFGSLAFVNAAAPYVSRLLGINIMIGHLVGVNLLTFWNPPVIV
HERNAAAEYLQLMFGINSMPPPGTRVGAAAVVLGVVFGINAIMPKAGLLINKTYQGSYGFKKAAARVRAMAP
AARYARDPQRFGAAAKLCPVQLWV

TABLE 17

Nucleic acid sequence
Polynucleotide #1 (SEQ ID NO: 159)
ATGGGAATGCAGGTGCAAATACAGTCTCTCTTCCTTTTGCTTCTCTGGGTTCCAGGATCACGGGGC
GTCCCCATTTCCCATCTCGATATTCTGAAGAAGCTGAGCGAGTACCTGCAACTGGTCGGCGCTGCAGCTATT
AGCCCTAGCTACACTTATTATCGGAAGGCTGCTGCTACCTATGCCTGTTTCGTGTCTAATCTCAAAGTCACA
GACTTCGGGCTCGCAAGAGGGGCTGCCGCTCACCTGTTCGGGTACTCTATCTATAAAAACGCCCAATATTC
CTGGTTTGTGAATGGAACCTTCAAAGCTGCAGCCAAGGTCTTCGGCAGCCTGGCATTTGTCAACGCCGCTG
CTCCCTACGTGAGCCGGCTCCTCGGGATTAATATTATGATCGGCCACCTGGTGGGAGTGAATCTGCTCACA
TTTTGGAACCCTCCAGTGATCGTGTACCCACCTCTCCATGAAAGGAACGCCGCAGCCGAATATCTGCAGCT
GATGTTCGGCATCAATGCCATTATGCCTAAAGCCGGACTGCTGATCAACAAGACTTACCAGGGCTCTTACGG
CTTCAAGAAGGCTGCAGCCCGCGTCAGAGCCATGGCTATCTACCGCAACGCCGCCGCTCGGTACGCCAGG
GACCCCCAGCGCTTTGGGGCTGCCGCCAAGCTGTGCCCAGTGCAGCTGTGGGTGAACGCTTCTATGCCCC
CTCCAGGCACAAGAGTGGGAGCCGCTGCTGTCGTGCTGGGAGTCGTGTTCGGCATCGCAAAGTTTGTGGC
CGCCTGGACCCTCAAGGCAGCAGCAAAAGTCGCAGAGATTGTGCACTTTCTGAACACTTACTCCCCCGCAC
TGAACAAAATGTTTAAAGCCGCATCCTATGGCTTCAGGCTGGGGTTCTTTAAGGCCGCCGCAAGCTCCTTCT
CTACCACAATCAATAAGAAGGCCGCTGCCGTGGTGTTCGGAATCCTCATCAAAAGATAG
Polynucleotide #2 (SEQ ID NO: 160)
ATGGGAATGCAGGTGCAAATACAGTCTCTCTTCCTTTTGCTTCTCTGGGTTCCAGGATCACGGGGC
ATTGTGTACCCCCCCCTGCACGAGCGGAACGCTGCTGCAGAATATCTCCAGCTCCTGTTCGGCATTAACGCCATTATGC
CTAAAGCAGGCCTGCTCATCAACAAAACTTACCAGGGAAGCTATGGGTTCAAGAAGGCAGCTGCAAGAGTCAGGGCCA
TGGCCATCTATCGGAATGCTGCAGCACGCTATGCCAGGGATCCTCAAAGGTTTGGGGCCGCCGCCAAGCTCTGTCCCG
TGCAACTCTGGGTCAATGCCTCCATGCCTCCACCCGGAACAAGAGTCGGAGCCGCCGCCGTGGTCCTCGGGGTGGTC
TTCGGGATCGCCAAATTCGTCGCCGCCTGGACACTGAAGGCCGCTGCTAAGGTCGCCGAACTGGTGTGGTTCCTGAAC
GCTGCCGCAACATACTCCCCTGCTCTCAACAAAATGTTTAAGGCTGCCTCTTACGGCTTTAGACTGGGATTTTTCAAGGG
AGCTGCCTCTAGCTTCTCTACAACTATCAATAAAAAGGCCGCAGCCGTCGTGTTCGGGATCCTGATCAAACGGGTGCCA
ATCAGCCATCTCGGCATCCTGAAGAAACTGTCTGAGTACCTGCAGCTGGTGGGGGCTGCCGCTATCTCTCCAAGCTAC
ACCTACTATAGAAAGGCAGCTGCTACCTACGCTTGCTTCGTGAGCAATCTGAAAGTGTTTGGCTCCCTGGCATTCGTCA
ACGCAGCTGCCCCATACGTGTCCCGCCTCCTGGGAATTAACGCTCACCTGTTTGGATATAGCGACTATAAGAATGCCCA
GTACTCCTGGTTCGTGAACGGCACCTTCAAGGCAGCCGCAAAGGTGACCGACTTTGGCCTGGCTCGCAACATTATGAT
GGGCCATCTGGTGGGCGTGAATCTCCTGACTTTTTGGAATCCCCCTGTGTAG
Polynucleotide #3 (SEQ ID NO: 161)
ATGGGAATGCAGGTGCAAATACAGTCTCTCTTCCTTTTGCTTCTCTGGGTTCCAGGATCACGGGGC
ACCTACTCTCCTGCACTCAATAAAATGTTTAAGGCCGCATCCTACGGCTTCAGGCTCGGATTCTTTAAGGCCGCCGCAAGCAGCTTT
TCTACTACAATCAACAAGAAAGCTGCCGCAGTGGTCTTTGGGATCCTCATCAAAAGGAACGCAGCCGCAGCTAAGTTCGTCGCTGC
TTGGACCCTGAAAGCCGCCGCCAAAGTCGCTGAAATCGTCCATTTCCTCAAGGTGACAGATTTTGGACTGGCTAGAGGCGCCGCC
GCTCACCTGTTCCCTTATTCCTGGTACAAAAACGCCACCTACGCTTGTTTCGTGAGCAACCTGAAGGCTGCCGCAGTGCCAATCTC
CCATCTCGAGATCCTGAAGAAACTGTCTGAGTACCTGCAGCTCGTCGGCGCCGCCGCAATTTCTCCCTCTTACACTTACTATCGCA
AAGCTGCCGCTCAATACAGCTGGTTTGTGAACGGAACTTTCAAGGCTGCCGCTAAGGTGTTCGGATCCCTGGCTTTCGTGAATGCC
GCCGCCCCCTATGTGAGCCGGCTGCTGGGAATTAATATTATGATTGGCCACCTGGTCGGAGTGAACCTGCTGACATTCTGGAATCC
TCGTGTGATTGTCTACCCACCTCTGCACGAAAGAAACGCCGCCGCCGAGTATCTCCAGCTCATGTTTGGGATCAATAGCATGCCAC
CCCCCGGCACCAGAGTGGGGGCAGCAGCCGTCGTCCTGGGCGTGGTGTTCGGGATCAACGCAATCATGCCAAAGGCCGGGCTG
CTGATTAACAAGACATACCAGGGGTCCTATGGCTTTAAGAAGGCCGCAGCTCGCGTGCGGGCTATGGCTATCTATAGGAATGCAGC
CGCTAGATATGCTCGCGACCCACAGCGGTTCGGCGCAGCTGCAAAGCTGTGCCCCGTGCAACTCTGGGTGTAG

TABLE 18

| | |
|---|---|
| Minigene #1 | (SEQ ID NO:) |

| | | | | | |
|---|---|---|---|---|---|
| 1 | CEA.691H5 | hc | IMIGHLVGV | (162) | A2 |
| 2 | CEA.24V9 | fa | LLTFWNPPV | (163) | A2 |
| 3 | HER2.369V2V9 | fa | KVFGSLAFV | (164) | A2 |
| 4 | HER2.665 | wt | VVLGVVFGI | (165) | A2 |
| 5 | p53.149M2 | fa | SMPPPGTRV | (166) | A2 |
| 6 | p53.139L2 | fa | KLCPVQLWV | (167) | A2 |
| 7 | MAGE3.112I5 | hc | KVAENHFL | (168) | A2 |
| 8 | MAGE2.153L2 | fa | KLSEYLQLV | (169) | A3 |
| 9 | CEA.61I7 | hc | HLFGYSIYK | (170) | A3 |
| 10 | CEA.420V2 | fa | ISPSYTYYR | (171) | A3 |
| 11 | HER2.860V2 | fa | KVTDFGLAR | (172) | A3 |
| 12 | HER2.669 | wt | VVFGILIKR | (173) | A3 |
| 13 | MAGE2.69K9 | fa | SSFSTTINK | (174) | A3 |
| 14 | MAGE2.299V2 | fa | IVYPPLHER | (175) | A3 |
| 15 | p53.101K10 | fa | KTYQGSYGFK | (176) | A3 |
| 16 | p53.156R9 | fa | RVRAMAIYR | (177) | A3 |
| 17 | CEA.268 | wt | QYSWFVNGTF | (178) | A24 |
| 18 | CEA.652 | wt | TYACFVSNL | (179) | A24 |
| 19 | HER2.780 | wt | PYVSRLLGI | (180) | A24 |
| 20 | HER2.978Y2 | fa | RYARDPQRF | (181) | A24 |
| 21 | MAGE2.156.M6 | hc | EYLQLMFGI | (182) | A24 |
| 22 | MAGE3.195 | wt | IMPKAGLLI | (183) | A24 |
| 23 | p53.106F9 | fa | SYGFRLGFF | (184) | A24 |
| 24 | p53.125 | wt | TYSPALNKMF | (185) | A24 |
| 25 | MAGE2.170D7 | hc | VPISHLDIL | (186) | B7 |

| | |
|---|---|
| Minigene #2 | (SEQ ID NO:) |

| | | | | | |
|---|---|---|---|---|---|
| 1 | CEA.691M3 | hc | IMMGVLVGV | (187) | A2 |
| 2 | CEA.24V9 | fa | LLTFWNPPV | (163) | A2 |
| 3 | HER2.369V2V9 | fa | KVFGSLAFV | (164) | A2 |
| 4 | HER2.665 | wt | VVLGVVFGI | (165) | A2 |
| 5 | p53.149M2 | fa | SMPPPGTRV | (166) | A2 |
| 6 | p53.139L2 | fa | KLCPVQLWV | (167) | A2 |
| 7 | MAGE3.112W7 | hc | KVAELVWFL | (188) | A2 |
| 8 | MAGE2.153L2 | fa | KLSEYLQLV | (169) | A3 |
| 9 | CEA.61D7 | hc | HLFGYSDYK | (189) | A3 |
| 10 | CEA.420V2 | fa | ISPSYTYYR | (171) | A3 |
| 11 | HER2.860V2 | fa | KVTDFGLAR | (172) | A3 |
| 12 | HER2.669 | wt | VVFGILIKR | (173) | A3 |
| 13 | MAGE2.69K9 | fa | SSFSTTINK | (174) | A3 |
| 14 | MAGE2.299V2 | fa | IVYPPLHER | (175) | A3 |
| 15 | p53.101K10 | fa | KTYQGSYGFK | (176) | A3 |
| 16 | p53.156R9 | fa | RVRAMAIYR | (177) | A3 |
| 17 | CEA.268 | wt | QYSWFVNGTF | (178) | A24 |
| 18 | CEA.652 | wt | TYACFVSNL | (179) | A24 |
| 19 | HER2.780 | wt | PYVSRLLGI | (180) | A24 |
| 20 | HER2.978Y2 | fa | RYARDPQRF | (181) | A24 |
| 21 | MAGE2.156L6 | hc | EYLQLLFGI | (190) | A24 |
| 22 | MAGE3.195 | wt | IMPKAGLLI | (183) | A24 |
| 23 | p53.106F9 | fa | SYGFRLGFF | (184) | A24 |
| 24 | p53.125 | wt | TYSPALNKMF | (185) | A24 |
| 25 | MAGE2.170G7 | hc | VPISHLGIL | (191) | B7 |

| | |
|---|---|
| Minigene #3 | (SEQ ID NO:) |

| | | | | | |
|---|---|---|---|---|---|
| 1 | CEA.691H5 | hc | IMIGHLVGV | (192) | A2 |
| 2 | CEA.24V9 | fa | LLTFWNPPV | (163) | A2 |
| 3 | HER2.369V2V9 | fa | KVFGSLAFV | (164) | A2 |
| 4 | HER2.665 | wt | VVLGVVFGI | (165) | A2 |
| 5 | p53.149M2 | fa | SMPPPGTRV | (166) | A2 |
| 6 | p53.139L2 | fa | KLCPVQLWV | (167) | A2 |
| 7 | MAGE3.112I5 | hc | KVAEIVHFL | (193) | A2 |
| 8 | MAGE2.153L2 | fa | KLSEYLQLV | (169) | A3 |
| 9 | CEA.61P4 | hc | HLFPYSWYK | (194) | A3 |
| 10 | CEA.420V2 | fa | ISPSYTYYR | (171) | A3 |
| 11 | HER2.860V2 | fa | KVTDFGLAR | (172) | A3 |
| 12 | HER2.669 | wt | VVFGILIKR | (173) | A3 |
| 13 | MAGE2.69K9 | fa | SSFSTTINK | (174) | A3 |
| 14 | MAGE2.299V2 | fa | IVYPPLHER | (175) | A3 |
| 15 | p53.101K10 | fa | KTYQGSYGFK | (176) | A3 |
| 16 | p53.156R9 | fa | RVRAMAIYR | (177) | A3 |
| 17 | CEA.268 | wt | QYSWFVNGTF | (178) | A24 |
| 18 | CEA.652 | wt | TYACFVSNL | (179) | A24 |
| 19 | HER2.780 | wt | PYVSRLLGI | (180) | A24 |
| 20 | HER2.978Y2 | fa | RYARDPQRF | (181) | A24 |
| 21 | MAGE2.156.M6 | hc | EYLQLMFGI | (195) | A24 |
| 22 | MAGE3.195 | wt | IMPKAGLLI | (183) | A24 |
| 23 | p53.106F9 | fa | SYGFRLGFF | (184) | A24 |
| 24 | p53.125 | wt | TYSPALNKMF | (185) | A24 |
| 25 | MAGE2.170E7 | hc | VPISHLEIL | (186) | B7 |

What is claimed is:

1. A method of producing a polypeptide comprising an analog of a Major Histocompatibility Complex (MHC) class I peptide epitope,
wherein said analog has enhanced immunogenicity compared to said epitope, said method comprising:
(a) identifying a MHC class I epitope comprising a formula (A),
wherein formula (A) is Rn-R2-R3-R4-R5-R6-R7- . . . Rx,
Rn is the N-terminal amino acid,
Rx is the C-terminal amino acid,
x=8-11 such that Rx can be from the eighth to the eleventh amino acid residue from Rn,
R2 or R3 and Rx are primary anchor residues of a motif or supermotif, and
(b) producing a polypeptide comprising an analog, said analog comprising a formula (B) identical to said formula (A) except one or more conservative or semiconservative amino acid substitutions at R3 and/or R5 and/or R7, provided said one or more substitutions is not of a primary anchor residues.

2. The method of claim 1, which comprises:
(a) identifying a MHC class I epitope comprising a formula (A),
wherein formula (A) is Rn-R2-R3-R4-R5-R6-R7- . . . Rx,
Rn is the N-terminal amino acid,
Rx is the C-terminal amino acid,
x=8-11 such that Rx can be from the eighth to the eleventh amino acid residue from Rn,
R2 or R3 and Rx are primary anchor residues of a motif or supermotif, and
R3 is Ile, and
(b) producing a polypeptide comprising an analog, said analog comprising a formula (B) identical to said formula (A) except that R3 is Met.

3. The method of claim 1, which comprises:
(a) identifying a MHC class I peptide epitope comprising a formula (A),
wherein formula(A) is Rn-R2-R3-R4-R5-R6-R7- . . . Rx,
Rn is the N-terminal amino acid,
Rx is the C-terminal amino acid,
x=8-11 such that Rx can be from the eighth to the eleventh amino acid residue from Rn,
R2 or R3 and Rx are primary anchor residues of a motif or supermotif, and
R7 is Tyr, and
(b) producing a polypeptide comprising an analog, said analog comprising a formula (B) identical to said formula (A) except that R7 is His.

4. The method of claim 1, which comprises:
(a) identifying a MHC class I peptide epitope comprising a formula (A), wherein formula (A) is Rn-R2-R3-R4-R5-R6-R7- . . . Rx,
Rn is the N-terminal amino acid,
Rx is the C-terminal amino acid,
x=8-11 such that Rx can be from the eighth to the eleventh amino acid residue from Rn,
R2 or R3 and Rx are primary anchor residues of a motif or supermotif, and
R7 is Tyr, and
(b) producing a polypeptide comprising an analog, said analog comprising a formula (B) identical to said formula (A) except that R7 is Met.

5. The method of claim 1, wherein the second Class I epitope exhibits at least about 50% increased potency for a specific T-cell compared to the first Class I epitope.

6. The method of claim 1, wherein only one substitution is introduced.

7. The method of claim 1, wherein the substitution is a conservative substitution.

8. The method of claim 1, wherein the substitution is a semi-conservative substitution.

9. The method of claim 1, wherein the peptide comprising said second Class 1 epitope induces both Th1 and Th2 cytokines when said peptide is bound by an HLA Class I molecule and contacted with a cytotoxic T-cell.

10. The method of claim 1, wherein the first Class I epitope comprises a supermotif selected from the group consisting of A1, A2, A3, A24, B7, B27, B44, B58 and B62.

11. The method of claim 1, wherein the first Class I epitope is derived from a viral antigen, a tumor-associated antigen, a parasitic antigen, a bacterial antigen or a fungal antigen.

12. A peptide comprising the second Class I epitope prepared by the method of claim 1.

13. A method to elicit an immune response which method comprises contacting cytotoxic T lymphocytes (CTLs) with the peptide of claim 9.

14. The method of claim 10, wherein the step of contacting is carried out in vitro in the presence of an antigen presenting cell.

15. The method of claim 10, wherein said contacting is carried out by administering to a subject a nucleic acid molecule comprising a nucleotide sequence encoding said peptide.

16. A composition comprising at least one peptide, the peptide comprising a Class I epitope obtainable by the method of claim 1.

17. The composition of claim 13, wherein the peptide contains 9-15 amino acids.

18. The composition of claim 13, wherein the peptide comprises an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:50, SEQ ID NO:51, SEQ ID NO:53, SEQ ID NO:54, SEQ ID NO:56, SEQ ID NO:58 and SEQ ID NO:59.

19. A composition of claim 13, wherein the peptide is admixed or joined to a CTL epitope.

20. A composition of claim 13, wherein the peptide is admixed or joined to an HTL epitope.

21. A composition of claim 17, wherein the HTL epitope is a pan-DR binding molecule.

22. A composition of claim 13, further comprising a liposome.

23. A composition of claim 13, wherein the epitope is coupled to a lipid.

24. A composition of claim 13, wherein said epitope is included in a heteropolymer.

25. A composition of claim 13, wherein the epitope is included in a homoplymer.

26. A composition of claim 13, wherein the epitope is bound to an HLA heavy chain, β2-microglobulin, and strepavidin complex, whereby a tetramer is formed.

27. A composition of claim 13, further comprising an antigen presenting cell, wherein the epitope is on or within the antigen presenting cell.

28. A composition of claim 24, wherein the epitope is bound to an HLA molecule on the antigen presenting cell, whereby when a cytotoxic lymphocyte (CTL) that is restricted to the HLA molecule is present, a receptor of the CTL binds to a complex of the HLA molecule and the epitope.

29. A composition of claim 25, wherein the antigen presenting cell is a dendritic cell.

30. A composition of claim 13, further comprising an HLA molecule, wherein the peptide is bound by the HLA molecule.

31. A composition of claim 13, further comprising a label.

32. A composition of claim 28, wherein the label is biotin, a fluorescent moiety, a non-mammalian sugar, a radio label or a small molecule to which a monoclonal antibody binds.

33. The composition of claim 13 which is a vaccine containing:
a unit dosage of said peptide, and a pharmaceutical excipient.

34. A nucleic acid molecule comprising a nucleotide sequence encoding a peptide of 9-15 amino acids which comprises a second Class I epitope obtainable by the method of claim 1.

35. The nucleic acid molecule of claim 31 wherein said peptide comprises an epitope consisting of an amino acid sequence selected from the group consisting of SEQ ID NO:2, SEQ ID NO:3, SEQ ID NO:5, SEQ ID NO:6, SEQ ID NO:8, SEQ ID NO:9, SEQ ID NO:50, SEQ ID NO:51, SEQ ID NO:53, SEQ ID NO:54, SEQ ID NO:56, SEQ ID NO:58 and SEQ ID NO:59.

36. The nucleic acid molecule of claim 32 which further comprises control sequences for expression of said nucleotide sequence.

37. A pharmaceutical composition which comprises as active ingredient the nucleic acid molecule of claim 31.

38. A method of producing a polypeptide comprising an analog of a Major Histocompatibility Complex (MHC) class I peptide epitope,
wherein said analog has enhanced immunogenicity compared to said epitope, said method comprising:
(a) identifying a MHC class I epitope comprising a formula (A),
wherein formula (A) is Rn-R2-R3-R4-R5-R6-R7- . . . Rx,
Rn is the N-terminal amino acid,
Rx is the C-terminal amino acid,
x=8-11 such that Rx can be from the eighth to the eleventh amino acid residue from Rn,
R2 and Rx are primary anchor residues of a B7 supermotif, and
(b) producing a polypeptide comprising an analog, said analog comprising a formula (B) identical to said formula (A) except that one or more than one of R3 and/or R5 and/or R7 comprise a non-conservative amino acid substitution.

39. The method of claim 35, which comprises:
(a) identifying a class I peptide epitope comprising a formula (A),
wherein formula(A) is Rn-R2-R3-R4-R5-R6-R7- . . . Rx,
Rn is the N-terminal amino acid,
Rx is the C-terminal amino acid,
x=8-11 such that Rx can be from the eighth to the eleventh amino acid residue from Rn,
R2 and Rx are primary anchor residues of a B7 supermotif, and
R7 is Tyr, and
producing a polypeptide comprising an analog, said analog comprising a formula (B) identical to said formula (A) except that Tyr is replaced with Gly, Glu, or Asp at R7.

40. A polypeptide comprising at least one heteroclitic analog of carcinoembryonic antigen (CEA) selected from the group consisting of:

| | | | |
|---|---|---|---|
| (a) | the heteroclitic | HLFPYSWYK; | (SEQ ID NO: 11) |
| | analog | | |
| (b) | the heteroclitic | HLFGYSLYK; | (SEQ ID NO: 13) |
| | analog | | |
| (c) | the heteroclitic | HLFGYSMYK; | (SEQ ID NO: 14) |
| | analog | | |
| (d) | the heteroclitic | HLFGYSIYK; | (SEQ ID NO: 15) |
| | analog | | |
| (e) | the heteroclitic | HLFGYSDYK; | (SEQ ID NO: 16) |
| | analog | | |
| (f) | the heteroclitic | HLFGYSGYK; | (SEQ ID NO: 17) |
| | analog | | |
| and | | | |
| (g) | the heteroclitic | HLFGYSCYK. | (SEQ ID NO: 18) |
| | analog | | |

41. The polypeptide of claim 40, which comprises more than one heteroclitic analog of CEA.

42. The polypeptide of claim 40 or 41, which comprises at least one peptide selected from the group consisting of the CEA epitopes and analogs in Tables 7, 13-15 and 18.

43. The polypeptide of any of claims 40-42, which comprises a fragment of CEA selected from the group consisting of:
(a) amino acids 61-69 of SEQ ID NO:68;
(b) amino acids 61-70, 61-71, 61-72, 61-73, 61-74, 61-75, 61-76, 61-77, 61-78, 61-79, 61-80, 61-81, 61-82, 61-83, 61-84, 61-85, 61-86, 61-87, 61-88, 61-89, 61-90, 61-91, 61-92, 61-93, 61-94, 61-95, 61-96, 61-97, 61-98, 61-99, 61-100, 61-101, 61-102, 61-103, 61-104, 61-105, 61-106, 61-107, 61-108, 61-109, 61-110, 61-111, 61-112, 61-113, 61-114, 61-115, 61-116, 61-117, 61-118, 61-119, 61-120, 61-121, 61-122, 61-123, 61-124, 61-125, 61-126, 61-127, 61-128, 61-129, 61-130, 61-131, 61-132, 61-133, 61-134, 61-135, 61-136, 61-137, 61-138, 61-139, 61-140, 61-141, 61-142, 61-143, 61-144, 61-145, 61-146, 61-147, 61-148, 61-149, 61-150, 61-151, 61-152, 61-153, 61-154, 61-155, 61-156, 61-157, 61-158, 61-159, 61-160, 61-161, 61-162, 61-163, 61-164, 61-165, 61-166, 61-167, 61-168, 61-169, 61-170, 61-171, 61-172, 61-173, 61-174, 61-175, 61-176, 61-177, 61-178, 61-179, 61-180, 61-181, 61-182, 61-183, 61-184, 61-185, 61-186, 61-187, 61-188, 61-189, 61-190, 61-191, 61-192, 61-193, 61-194, 61-195, 61-196, 61-197, 61-198, 61-199, 61-200, 61-201, 61-202, 61-203, 61-204, 61-205, 61-206, 61-207, 61-208, 61-209, 61-210, 61-211, 61-212, 61-213, 61-214, 61-215, 61-216, 61-217, 61-218, 61-219, 61-220, 61-221, 61-222, 61-223, 61-224, 61-225, 61-226, 61-227, 61-228, 61-229, 61-230, 61-231, 61-232, 61-233, 61-234, 61-235, 61-236, 61-237, 61-238, 61-239, 61-240, 61-241, 61-242, 61-243, 61-244, 61-245, 61-246, 61-247, 61-248, 61-249, 61-250, 61-251, 61-252, 61-253, 61-254, 61-255, 61-256, 61-257, 61-258, 61-259, 61-260, 61-261, 61-262, 61-263, 61-264, 61-265, 61-266, 61-267, 61-268, 61-269, 61-270, 61-271, 61-272, 61-273, 61-274, 61-275, 61-276, 61-277, 61-278, 61-279, 61-280, 61-281, 61-282, 61-283, 61-284, 61-285, 61-286, 61-287, 61-288, 61-289, 61-290, 61-291, 61-292, 61-293, 61-294, 61-295, 61-296, 61-297, 61-298, 61-299, 61-300, 61-301, 61-302, 61-303, 61-304, 61-305, 61-306, 61-307, 61-308, 61-309, 61-310, 61-311, 61-312, 61-313, 61-314, 61-315, 61-316, 61-317, 61-318, 61-319, 61-320, 61-321, 61-322, 61-323, 61-324, 61-325, 61-326, 61-327, 61-328, 61-329, 61-330, 61-331, 61-332, 61-333, 61-334, 61-335, 61-336, 61-337, 61-338, 61-339, 61-340, 61-341, 61-342, 61-343, 61-344, 61-345, 61-346, 61-347, 61-348, 61-349, 61-350, 61-351, 61-352, 61-353, 61-354, 61-355, 61-356, 61-357, 61-358, 61-359, 61-360, 61-361, 61-362, 61-363, 61-364, 61-365, 61-366, 61-367, 61-368, 61-369, 61-370, 61-371, 61-372, 61-373, 61-374, 61-375, 61-376, 61-377, 61-378, 61-379, 61-380, 61-381, 61-382, 61-383, 61-384, 61-385, 61-386, 61-387, 61-388, 61-389, 61-390, 61-391, 61-392, 61-393, 61-394, 61-395, 61-396, 61-397, 61-398, 61-399, 61-400, 61-401, 61-402, 61-403, 61-404, 61-405, 61-406, 61-407, 61-408, 61-409, 61-410, 61-411, 61-412, 61-413, 61-414, 61-415, 61-416, 61-417, 61-418, 61-419, 61-420, 61-421, 61-422, 61-423, 61-424, 61-425, 61-426, 61-427, 61-428, 61-429, 61-430, 61-431, 61-432, 61-433, 61-434, 61-435, 61-436, 61-437, 61-438, 61-439, 61-440, 61-441, 61-442, 61-443, 61-444, 61-445, 61-446, 61-447, 61-448, 61-449, 61-450, 61-451, 61-452, 61-453, 61-454, 61-455, 61-456, 61-457, 61-458, 61-459, 61-460, 61-461, 61-462, 61-463, 61-464, 61-465, 61-466, 61-467, 61-468, 61-469, 61-470, 61-471, 61-472, 61-473, 61-474, 61-475, 61-476, 61-477, 61-478, 61-479, 61-480, 61-481, 61-482, 61-483, 61-484, 61-485, 61-486, 61-487, 61-488, 61-489, 61-490, 61-491, 61-492, 61-493, 61-494, 61-495, 61-496, 61-497, 61-498, 61-499, 61-500, 61-501, 61-502, 61-503, 61-504, 61-505, 61-506, 61-507, 61-508, 61-509, 61-510, 61-511, 61-512, 61-513, 61-514, 61-515, 61-516, 61-517, 61-518, 61-519, 61-520, 61-521, 61-522, 61-523, 61-524, 61-525, 61-526, 61-527, 61-528, 61-529, 61-530, 61-531, 61-532, 61-533, 61-534, 61-535, 61-536, 61-537, 61-538, 61-539, 61-540, 61-541, 61-542, 61-543, 61-544, 61-545, 61-546, 61-547, 61-548, 61-549, 61-550, 61-551, 61-552, 61-553, 61-554, 61-555, 61-556, 61-557, 61-558, 61-559, 61-560, 61-561, 61-562, 61-563, 61-564, 61-565, 61-566, 61-567, 61-568, 61-569, 61-570, 61-571, 61-572, 61-573, 61-574, 61-575, 61-576, 61-577, 61-578, 61-579, 61-580, 61-581, 61-582, 61-583, 61-584, 61-585, 61-586, 61-587, 61-588, 61-589, 61-590, 61-591, 61-592, 61-593, 61-594, 61-595, 61-596, 61-597, 61-598, 61-599, 61-600, 61-601, 61-602, 61-603, 61-604, 61-605, 61-606, 61-607, 61-608, 61-609, 61-610, 61-611, 61-612, 61-613, 61-614, 61-615, 61-616, 61-617, 61-618, 61-619, 61-620, 61-621, 61-622, 61-623, 61-624, 61-625, 61-626, 61-627, 61-628, 61-629, 61-630, 61-631, 61-632, 61-633, 61-634, 61-635, 61-636, 61-637, 61-638, 61-639, 61-640, 61-641, 61-642, 61-643, 61-644, 61-645, 61-646, 61-647, 61-648, 61-649, 61-650, 61-651, 61-652, 61-653, 61-654, 61-655, 61-656, 61-657, 61-658, 61-659, 61-660, 61-661, 61-662, 61-663, 61-664, 61-665, 61-666, 61-667, 61-668, 61-669, 61-670, 61-671, 61-672, 61-673, 61-674, 61-675, 61-676, 61-677, 61-678, 61-679, 61-680, 61-681, 61-682, 61-683, 61-684, 61-685, 61-686, 61-687, 61-688, 61-689, 61-690, 61-691, 61-692, 61-693, 61-694, 61-695, 61-696, 61-697, 61-698, 61-699, 61-700, 61-701, and 61-702 of SEQ ID NO:68; and
(c) amino acids 1-69, 2-69, 3-69, 4-69, 5-69, 6-69, 7-69, 8-69, 9-69, 10-69, 11-69, 12-69, 13-69, 14-69, 15-69, 16-69, 17-69, 18-69, 19-69, 20-69, 21-69, 22-69, 23-69, 24-69, 25-69, 26-69, 27-69, 28-69, 29-69, 30-69, 31-69, 32-69, 33-69, 34-69, 35-69, 36-69, 37-69, 38-69, 39-69, 40-69, 41-69, 42-69, 43-69, 44-69, 45-69, 46-69, 47-69, 48-69, 49-69, 50-69, 51-69, 52-69, 53-69, 54-69, 55-69, 56-69, 57-69, 58-69, 59-69, 60-69, of SEQ ID NO:68.

44. A polypeptide comprising at least one heteroclitic analog of melanoma antigen 2 (MAGE2) selected from the group consisting of:
(a) the heteroclitic analog EYIQLVFGI (SEQ ID NO:21);
(b) the heteroclitic analog EYLELVFGI (SEQ ID NO:22);
(c) the heteroclitic analog EYLLLVFGI (SEQ ID NO:23);
(d) the heteroclitic analog EYLQLMFGI (SEQ ID NO:24); and
(e) the heteroclitic analog EYLQLLFGI (SEQ ID NO:25).

45. The polypeptide of claim 44, which comprises more than one heteroclitic analog of MAGE2.

46. The polypeptide of claim 44 or 45, which comprises at least one peptide selected from the group consisting of the MAGE2 epitopes and analogs in Tables 7, 8, 13-15 and 18.

47. The polypeptide of any of claims 44-46, which comprises a fragment of MAGE2 selected from the group consisting of:
(a) amino acids 157-163-69 of SEQ ID NO:69;
(b) 1-163, 2-163, 3-163, 4-163, 5-163, 6-163, 7-163, 8-163, 9-163, 10-163, 11-163, 12-163, 13-163, 14-163, 15-163, 16-163, 17-163, 18-163, 19-163, 20-163, 21-163, 22-163, 23-163, 24-163, 25-163, 26-163, 27-163, 28-163, 29-163, 30-163, 31-163, 32-163, 33-163, 34-163, 35-163, 36-163, 37-163, 38-163, 39-163, 40-163, 41-163, 42-163, 43-163, 44-163, 45-163, 46-163, 47-163, 48-163, 49-163, 50-163, 51-163, 52-163, 53-163, 54-163, 55-163, 56-163, 57-163, 58-163, 59-163, 60-163, 61-163, 62-163, 63-163, 64-163, 65-163, 66-163, 67-163, 68-163, 69-163, 70-163, 71-163, 72-163, 73-163, 74-163, 75-163, 76-163, 77-163, 78-163, 79-163, 80-163, 81-163, 82-163, 83-163, 84-163, 85-163, 86-163, 87-163, 88-163, 89-163, 90-163, 91-163, 92-163, 93-163, 94-163, 95-163, 96-163, 97-163, 98-163, 99-163, 100-163, 101-163, 102-163, 103-163, 104-163, 105-163, 106-163, 107-163, 108-163, 109-163, 110-163, 111-163, 112-163, 113-163, 114-163, 115-163, 116-163, 117-163, 118-163, 119-163, 120-163, 121-163, 122-163, 123-163, 124-163, 125-163, 126-163, 127-163, 128-163, 129-163, 130-163, 131-163, 132-163, 133-163, 134-163, 135-163, 136-163, 137-163, 138-163, 139-163, 140-163, 141-163, 142-163, 143-163, 144-163, 145-163, 146-163, 147-163, 148-163, 149-163, 150-163, 151-163, 152-163, 153-163, 154-163, 155-163, 156-163 of SEQ ID NO:69; and
(c) amino acids 157-164, 165, 157-166, 157-167, 157-168, 157-169, 157-170, 157-171, 157-172, 157-173, 157-174, 157-175, 157-176, 157-177, 157-178, 157-179, 157-180, 157-181, 157-182, 157-183, 157-184, 157-185, 157-186, 157-187, 157-188, 157-189, 157-190, 157-191, 157-192, 157-193, 157-194, 157-195, 157-196, 157-197, 157-198, 157-199, 157-200, 157-201, 157-202, 157-203, 157-204, 157-205, 157-206, 157-207, 157-208, 157-209, 157-210, 157-211, 157-212, 157-213, 157-214, 157-215, 157-216, 157-217, 157-218, 157-219, 157-220, 157-221, 157-222, 157-223, 157-224, 157-225, 157-226, 157-227, 157-228, 157-229, 157-230, 157-231, 157-232, 157-233, 157-234, 157-235, 157-236, 157-237, 157-238, 157-239, 157-240, 157-241, 157-242, 157-243, 157-244, 157-245, 157-246, 157-247, 157-248, 157-249, 157-250, 157-251, 157-252, 157-253, 157-254, 157-255, 157-256, 157-257, 157-258, 157-259, 157-260, 157-261, 157-262, 157-263, 157-264, 157-265, 157-266, 157-267, 157-268, 157-269, 157-270, 157-271, 157-272, 157-273, 157-274, 157-275, 157-276, 157-277, 157-278, 157-279, 157-280, 157-281, 157-282, 157-283, 157-284, 157-285, 157-286, 157-287, 157-288, 157-289, 157-290, 157-291, 157-292, 157-293, 157-294, 157-295, 157-296, 157-297, 157-298, 157-299, 157-300, 157-301, 157-302, 157-303, 157-304, 157-305, 157-306, 157-307, 157-308, 157-309, 157-310, 157-311, 157-312, 157-313, 157-314 of SEQ ID NO:69.

48. The polypeptide of any of claims 40-47, which comprises at least one peptide selected from the group consisting of the p53, CEA, MAGE2, MAGE3, Her2/neu, epitopes and analogs in Tables 13-15 and 18.

49. The polypeptide of any of claims 40-48, which comprises at least one T helper peptide.

50. The polypeptide of claim 49, wherein at least one of said at least one T helper peptide is selected from the group consisting of: aKXVAAWTLKAAa, where “X” is either cyclohexylalanine (SEQ ID NO:29), phenylalanine (SEQ ID NO:30), or tyrosine (SEQ ID NO:31), and “a” is either D-alanine or L-alanine.

51. The polypeptide of any of claims 40-50, which comprises at least one spacer.

52. The polypeptide of any of claims 40-51, which comprises at least one leader.

53. A polynucleotide encoding the polypeptide of any of claims 40-52.

54. A composition comprising at least one polypeptide of any of claims 40-52 or at least one polynucleotide of claim 53 and at least one component selected from the group consisting of: an excipient; an adjuvant; and a lipid.

55. A composition comprising two or more polypeptides of any of claims 40-52.

56. A composition comprising two or more polynucleotides of claim 53.

57. The composition of claim 55 or 56, which comprises at least one component selected from the group consisting of: and excipient; and adjuvant; and a lipid.

58. The composition of any of claims 54-57, which comprises an HLA heavy chain, β2-microglobulin, and streptavidin.

59. The composition of claim 58, wherein said polypeptide is bound to a complex of said HLA heavy chain, β2-microglobulin, and streptavidin, whereby a tetramer is formed.

60. The composition of any of claims 54-58, comprising an antigen presenting cell, wherein the polypeptide is on or within the antigen presenting cell.

61. The composition of claim 60, wherein the polypeptide is bound to an HLA molecule on the antigen presenting cell, whereby when a cytotoxic T lymphocyte (CTL) that is restricted to the HLA molecule is present, a receptor of the CTL binds to a complex of the HLA molecule and the epitope.

62. The polynucleotide of claim 53, which comprises at least one control sequence.

63. A composition comprising the polynucleotide of claim 62 and a component selected from the group consisting of: an excipient; an adjuvant; and a lipid.


SEQUENCE LISTING (196 sequences)

SEQ ID NO: 1 - protein, 9 aa, Artificial Sequence
  Synthetic peptide derived from Homo sapiens carcinoembryonic antigen
IMIGVLVGV

SEQ ID NO: 2 - protein, 9 aa, Artificial Sequence
  Synthetic peptide derived from Homo sapiens carcinoembryonic antigen
IMMGVLVGV

SEQ ID NO: 3 - protein, 9 aa, Artificial Sequence
  Synthetic peptide derived from Homo sapiens carcinoembryonic antigen
IMIGHLVGV

SEQ ID NO: 4 - protein, 9 aa, Artificial Sequence
  Synthetic peptide derived from Homo sapiens melanoma antigens
KVAELVHFL

SEQ ID NO: 5 - protein, 9 aa, Artificial Sequence
  Synthetic peptide derived from Homo sapiens melanoma antigens
KVAEIVHFL

SEQ ID NO: 6 - protein, 9 aa, Artificial Sequence
  Synthetic peptide derived from Homo sapiens melanoma antigens
KVAELVWFL

SEQ ID NO: 7 - protein, 10 aa, Artificial Sequence
  Synthetic peptide derived from Homo sapiens melanoma antigens
YLQLVFGIEV

SEQ ID NO: 8 - protein, 10 aa, Artificial Sequence
  Synthetic peptide derived from Homo sapiens melanoma antigens
YLQLIFGIEV

SEQ ID NO: 9 - protein, 10 aa, Artificial Sequence
  Synthetic peptide derived from Homo sapiens melanoma antigens
YLQLFFGIEV

SEQ ID NO: 10 - protein, 9 aa, Artificial Sequence
  Synthetic peptide derived from Homo sapiens carcinoembronic antigen
HLFGYSWYK

SEQ ID NO: 11 - protein, 9 aa, Artificial Sequence
  Synthetic peptide derived from Homo sapiens carcinoembronic antigen
HLFPYSWYK

SEQ ID NO: 12 - protein, 9 aa, Artificial Sequence
  Synthetic peptide derived from Homo sapiens carcinoembronic antigen
HLFIYSWYK

SEQ ID NO: 13 - protein, 9 aa, Artificial Sequence
  Synthetic peptide derived from Homo sapiens carcinoembronic antigen
HLFGYSLYK

SEQ ID NO: 14 - protein, 9 aa, Artificial Sequence
  Synthetic peptide derived from Homo sapiens carcinoembronic antigen
HLFGYSMYK

SEQ ID NO: 15 - protein, 9 aa, Artificial Sequence
  Synthetic peptide derived from Homo sapiens carcinoembronic antigen
HLFGYSIYK

SEQ ID NO: 16 - protein, 9 aa, Artificial Sequence
  Synthetic peptide derived from Homo sapiens carcinoembronic antigen
HLFGYSDYK

SEQ ID NO: 17 - protein, 9 aa, Artificial Sequence
  Synthetic peptide derived from Homo sapiens carcinoembronic antigen
HLFGYSGYK

SEQ ID NO: 18 - protein, 9 aa, Artificial Sequence
  Synthetic peptide derived from Homo sapiens carcinoembronic antigen
HLFGYSCYK

SEQ ID NO: 19 - protein, 9 aa, Artificial Sequence
  Synthetic peptide derived from Homo sapiens carcinoembronic antigen
HLFGYSNYK

SEQ ID NO: 20 - protein, 9 aa, Artificial Sequence
  Synthetic peptide derived from Homo sapiens melanoma antigens
EYLQLVFGI

SEQ ID NO: 21 - protein, 9 aa, Artificial Sequence
  Synthetic peptide derived from Homo sapiens melanoma antigens
EYIQLVFGI

SEQ ID NO: 22 - protein, 9 aa, Artificial Sequence
  Synthetic peptide derived from Homo sapiens melanoma antigens
EYLELVFGI

SEQ ID NO: 23 - protein, 9 aa, Artificial Sequence
  Synthetic peptide derived from Homo sapiens melanoma antigens
EYLLLVFGI

SEQ ID NO: 24 - protein, 9 aa, Artificial Sequence
  Synthetic peptide derived from Homo sapiens melanoma antigens
EYLQLMFGI

SEQ ID NO: 25 - protein, 9 aa, Artificial Sequence
  Synthetic peptide derived from Homo sapiens melanoma antigens
EYLQLLFGI

SEQ ID NO: 26 - protein, 14 aa, Artificial Sequence
  Synthetic peptide derived from Clostridium tetani
QYIKANSKFIGITE

SEQ ID NO: 27 - protein, 21 aa, Artificial Sequence
  Synthetic peptide derived from Plasmodium falciparum
DIEKKIAKMEKASSVFNVVNS

SEQ ID NO: 28 - protein, 16 aa, Artificial Sequence
  Synthetic peptide derived from Streptococcus sp.
GAVDSILGGVATYGAA

SEQ ID NO: 29 - protein, 13 aa, Unknown
  Synthetic T helper peptide
AKXVAAWTLKAAA

SEQ ID NO: 30 - protein, 13 aa, Unknown
  Synthetic T helper peptide
AKXVAAWTLKAAA

SEQ ID NO: 31 - protein, 13 aa, Unknown
  Synthetic T helper peptide
AKXVAAWTLKAAA

SEQ ID NO: 32 - protein, 9 aa, Unknown
  Synthetic peptide
APAAAAAAY

SEQ ID NO: 33 - protein, 10 aa, Artificial Sequence
  Synthetic peptide derived from Homo sapiens Human Leukocyte Antigen
KVFPYALINK

SEQ ID NO: 34 - protein, 13 aa, Artificial Sequence
  Synthetic peptide derived from Hepatitis B Virus
TPPAYRPPNAPIL

SEQ ID NO: 35 - protein, 10 aa, Artificial Sequence
  Synthetic peptide derived from Hepatitis B Virus
FLPSDFFPSV

SEQ ID NO: 36 - protein, 9 aa, Artificial Sequence
  Synthetic peptide derived from Homo sapiens Human Leukocyte Antigen
APRTLVYLL

SEQ ID NO: 37 - protein, 9 aa, Artificial Sequence
  Synthetic peptide derived from Homo sapiens Human Leukocyte Antigen
APETLVYLL

SEQ ID NO: 38 - protein, 9 aa, Artificial Sequence
  Synthetic peptide derived from Homo sapiens Human Leukocyte Antigen
APRTWVYLL

SEQ ID NO: 39 - protein, 9 aa, Artificial Sequence
  Synthetic peptide derived from Homo sapiens Human Leukocyte Antigen
APRTLVPLL

SEQ ID NO: 40 - protein, 10 aa, Artificial Sequence
  Synthetic peptide derived from Homo sapiens Human Leukocyte Antigen
KVHPYALINK

SEQ ID NO: 41 - protein, 10 aa, Artificial Sequence
  Synthetic peptide derived from Homo sapiens Human Leukocyte Antigen
KVFPQALINK

SEQ ID NO: 42 - protein, 10 aa, Artificial Sequence
  Synthetic peptide derived from Homo sapiens Human Leukocyte Antigen
KVFPYAKINK

SEQ ID NO: 43 - protein, 9 aa, Artificial Sequence
  Synthetic peptide derived from Homo sapiens melanoma antigens
VPISHLYIL

SEQ ID NO: 44 - protein, 9 aa, Artificial Sequence
  Synthetic peptide derived from Homo sapiens melanoma antigens
VPISHLHIL

SEQ ID NO: 45 - protein, 9 aa, Artificial Sequence
  Synthetic peptide derived from Homo sapiens melanoma antigens
VPISHLMIL

SEQ ID NO: 46 - protein, 9 aa, Artificial Sequence
  Synthetic peptide derived from Homo sapiens melanoma antigens
VPISHLGIL

SEQ ID NO: 47 - protein, 9 aa, Artificial Sequence
  Synthetic peptide derived from Homo sapiens melanoma antigens
VPISHLEIL

SEQ ID NO: 48 - protein, 9 aa, Artificial Sequence
  Synthetic peptide derived from Homo sapiens melanoma antigens
VPISHLDIL

SEQ ID NO: 49 - protein, 9 aa, Artificial Sequence
  Synthetic peptide derived from Homo sapiens p53
SMPPPGTRV

SEQ ID NO: 50 - protein, 9 aa, Artificial Sequence
  Synthetic peptide derived from Homo sapiens p53
CMPPPGTRV

SEQ ID NO: 51 - protein, 9 aa, Artificial Sequence
  Synthetic peptide derived from Homo sapiens p53
SMPPPGPRV

SEQ ID NO: 52 - protein, 11 aa, Artificial Sequence
  Synthetic peptide derived from Homo sapiens p53
GLAPPQHLIRV

SEQ ID NO: 53 - protein, 11 aa, Artificial Sequence
  Synthetic peptide derived from Homo sapiens p53
GLTPPQHLIRV

SEQ ID NO: 54 - protein, 11 aa, Artificial Sequence
  Synthetic peptide derived from Homo sapiens p53
GLTPPEHLIRV

SEQ ID NO: 55 - protein, 9 aa, Artificial Sequence
  Synthetic peptide derived from Hepatitis B Virus
GLSRYVARL

SEQ ID NO: 56 - protein, 9 aa, Artificial Sequence
  Synthetic peptide derived from Hepatitis B Virus
GLSRYVPRL

SEQ ID NO: 57 - protein, 9 aa, Artificial Sequence
  Synthetic peptide derived from Human Immunodeficiency Virus
ILKEPVHGV

SEQ ID NO: 58 - protein, 9 aa, Artificial Sequence
  Synthetic peptide derived from Human Immunodeficiency Virus
ILHEPVHGV

SEQ ID NO: 59 - protein, 9 aa, Artificial Sequence
  Synthetic peptide derived from Human Immunodeficiency Virus
ILLEPVHGV

SEQ ID NO: 60 - protein, 9 aa, Artificial Sequence
  Synthetic peptide derived from Homo sapiens p53
LLGRDSFEV

SEQ ID NO: 61 - protein, 9 aa, Artificial Sequence
  Synthetic peptide derived from Homo sapiens p53
LLDRDSFEV

SEQ ID NO: 62 - protein, 9 aa, Artificial Sequence
  Synthetic peptide derived from Homo sapiens p53
LLHRDSFEV

SEQ ID NO: 63 - protein, 9 aa, Artificial Sequence
  Synthetic peptide derived from Homo sapiens p53
LLGRDSLEV

SEQ ID NO: 64 - protein, 9 aa, Artificial Sequence
  Synthetic peptide derived from Homo sapiens p53
LLGRDSHEV

SEQ ID NO: 65 - protein, 9 aa, Artificial Sequence
  Synthetic peptide derived from Homo sapiens p53
LLGRNSFEV

SEQ ID NO: 66 - protein, 9 aa, Artificial Sequence
  Synthetic peptide derived from Homo sapiens p53
LLGRGSFEV

SEQ ID NO: 67 - protein, 9 aa, Artificial Sequence
  Synthetic peptide derived from Human Immunodeficiency Virus
ILIEPVHGV

SEQ ID NO: 68 - protein, 702 aa, Homo sapiens [sequence omitted]

SEQ ID NO: 69 - protein, 314 aa, Homo sapiens
MPLEQRSQHCKPEEGLEARGEALGLVGAQAPATEEQQTASSSSTLVEVTLGEVPAADSPSPPHSPQGASSFSTTINYTLWRQSDEGSSNQEEEGPRMFPDLESEFQAAISRKMVELVHFLLLKYRAREPVTKAEMLESVLRNCQDFFPVIFSKASEYLQLVFGIEVVEVVPISHLYILVTCLGLSYDGLLGDNQVMPKTGLLIIVLAIIAIEGDCAPEEKIWEELSMLEVFEGREDSVFAHPRKLLMQDLVQENYLEYRQVPGSDPACYEFLWGPRALIETSYVKVLHHTLKIGGEPHISYPPLHERALREGEE

SEQ ID NO: 70 - protein, 609 aa, Homo sapiens [sequence omitted]

SEQ ID NO: 71 - protein, 212 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 72 - protein, 2768 aa, Homo sapiens
  misc_feature (734)..(734): Xaa can be any naturally occurring amino acid
MALVLEIFTLLASICWVSANIFEYQVDAQPLRPCELQRETAFLKQADYVPQCAEDGSFQTVQCQNDGRSCWCVGANGSEVLGSRQPGRPVACLSFCQLQKQQILLSGYINSTDTSYLPQCQDSGDYAPVQCDVQQVQCWCVDAEGMEVYGTRQLGRPKRCPRSCEIRNRRLLHGVGDKSPPQCSAEGEFMPVQCKFVNTTDMMIFDLVHSYNRFPDAFVTFSSFQRRFPEVSGYCHCADSQGRELAETGLELLLDEIYDTIFAGLDLPSTFTETTLYRILQRRFLAVQSVISGRFRCPTKCEVERFTATSFGHPYVPSCRRNGDYQAVQCQTEGPCWCVDAQGKEMHGTRQQGEPPSCAEGQSCASERQQALSRLYFGTSGYFSQHDLFSSPEKRWASPRVARFATSCPPTIKELFVDSGLLRPMVEGQSQQFSVSENLLKEAIRAIFPSRGLARLALQFTTNPKRLQQNLFGGKFLVNVGQFNLSGALGTRGTFNFSQFFQQLGLASFLNGGRQEDLAKPLSVGLDSNSSTGTPEAAKKDGTMNKPTVGSFGFEINLQENQNALKFLASLLELPEFLLFLQHAISVPEDVARDLGDVMETVLSSQTCEQTPERLFVPSCTTEGSYEDVQCFSGECWCVNSWGKELPGSRVRGGQPRCPTDCEKQRARMQSLMGSQPAGSTLFVPACTSEGHFLPVQCFNSECYCVDAEGQAIPGTRSAIGKPKKCPTPCQLQXEQAFLRTVQALLSNSSMLPTLSDTYIPQCSTDGQWRQVQCNGPPEQVFELYQRWEAQNKGQDLTPAKLLVKIMSYREAASGNFSLFIQSLYEAGQQDVFPVLSQYPSLQDVPLAALEGKRPQPRENILLEPYLFWQILNGQLSQYPGSYSDFSTPLAHFDLRNCWCVDEAGQELEGMRSEPSKLPTCPGSCEEAKLRVLQFIRETEEIVSASNSSRFPLGESFLVAKGIRLRNEDLGLPPLFPPREAFAEQFLRGSDYAIRLAAQSTLSFYQRRRFSPDDSAGASALLRSGPYXPQCDAFGSWEPVQCHAGTGHCWCVDEKGGFIPGSLTARSLQIPQCPTTCEKSRTSGLLSSWKQARSQENPSPKDLFVPACLETGEYARLQASGAGTWCVDPASGEELRPGSSSSAQCPSLCNVLKSGVLSRRVSPGYVPACRAEDGGFSPVQCDQAQGSCWCVMDSGEEVPGTRVTGGQPACESPRCPLPFNASEVVGGTILCETISGPTGSAMQQCQLLCRQGSWSVFPPGPLICSLESGRWESQLPQPRACQRPQLWQTIQTQGHFQLQLPPGKMCSADYAGLLQTFQVFILDELTARGFCQIQVKTFGTLVSIPVCNNSSVQVGCLTRERLGVNVTWKSRLEDIPVASLPDLHDIERALVGKDLLGRFTDLIQSGSFQLHLDSKTFPAETIRFLQGDHFGTSPRTWFGCSEGFYQVLTSEASQDGLGCVKCPEGSYSQDEECIPCPVGFYQEQAGSLACVPCPVGRTTISAGAFSQTHCVTDCQRNEAGLQCDQNGQYRASQKDRGSGKAFCVDGEGRRLPWWETEAPLEDSQCLMMQKFEKVPESKVIFDANAPVAVRSKVPDSEFPVMQCLTDCTEDEACSFFTVSTTEPEISCDFYAWTSDNVACMTSDQKRDALGNSKATSFGSLRCQVKVRSHGQDSPAVYLKKGQGSTTTLQKRFEPTGFQNMLSGLYNPIVFSASGANLTDAHLFCLLACDRDLCCDGFVLTQVQGGAIICGLLSSPSVLLCNVKDWMDPSEAWANATCPGVTYDQESHQVILRLGDQEFIKSLTPLEGTQDTFTNFQQVYLWKDSDMGSRPESMGCRKBTVPRPASPTEAGLTTELFSPVDLNQVIVNGNQSLSSQKHWLFKHLFSAQQANLWCLSRCVQEHSFCQLAEITESASLYFTCTLYPEAQVCDDIMESNAQGCRLILPQMPKALFRKKVILEDKVKNFYTRLPFQKLMGISIRNKVPMSEKSISNGFFECERRCDADPCCTGFGFLNVSQLKGGEVTCLTLNSLGIQMCSEENGGAWRILDCGSPDIEVHTYPFGWYQKPIAQNNAPSFCPLVVLPSLTEKVSLDSWQSLALSSVVVDPSIRHFDVAHVSTAATSNFSAVRDLCLSECSQHEACLITTLQTQPGAVRCMFYADTQSCTHSLQGQNCRLLLREEATHIYRKPGISLLSYEASVPSVPISTHGRLLGRSQAIQVGTSWKQVDQFLGVPYAAPPLAERRFQAPEPLNWTGSWDASKPRASCWQPGTRTSTSPGVSEDCLYLNVFIPQNVAPNASVLVFFHNTMDREESEGWPAIDGSFLAAVGNLIVVTASYRVGVFGFLSSGSGEVSGNWGLLDQVAALTWVQTHIRGFGGDPRRVSLAADRGGADVASIHLLTARATNSQLFRRAVLMGGSALSPAAVISHERAQQQAIALAKEVSCPMSSSQEVVSCLRQKPANVLNDAQTKLLAVSGPFHYWGPVIDGHFLREPPARALKRSLXVEVDLLIGSSQDDGLINRAKAVKQFEESQGRTSSKTAFYQALQNSLGGEDSDARVEAAATWYYSLEHSTDDYASFSRALENATRDYFIICPIIDMASAWAKRARGNVFMYHAPENYGHGSLELLADVQFALGLPFYPAYEGQFSLEEKSLSLKIMQYFSHFIRSGNPNYPYEFSRKVPTFATPWPDFVPRAGGENYKEFSELLPNRQGLKKADCSFWSKYISSLKTSADGAKGGQSAESEEEELTAGSGLREDLLSLQEPGSKTYSK

SEQ ID NO: 73 - protein, 10 aa, Artificial Sequence
  Synthetic peptide derived from Homo sapiens carcinoembryonic antigen
TISPLNTSYK

SEQ ID NO: 74 - protein, 10 aa, Artificial Sequence
  Synthetic peptide derived from Homo sapiens carcinoembryonic antigen
RTLTLLSVTR

SEQ ID NO: 75 - protein, 9 aa, Artificial Sequence
  Synthetic peptide derived from Homo sapiens carcinoembryonic antigen
IVPSYTYYR

SEQ ID NO: 76 - protein, 10 aa, Artificial Sequence
  Synthetic peptide derived from Homo sapiens carcinoembryonic antigen
RTLTLFNVTR

SEQ ID NO: 77 - protein, 9 aa, Artificial Sequence
  Synthetic peptide derived from Homo sapiens carcinoembryonic antigen
HTQVLFIAK

SEQ ID NO: 78 - protein, 9 aa, Artificial Sequence
  Synthetic peptide derived from Homo sapiens carcinoembryonic antigen
FVSNLATGR

SEQ ID NO: 79 - protein, 8 aa, Artificial Sequence
  Synthetic peptide derived from Homo sapiens melanoma antigens
SSFTTINK

SEQ ID NO: 80 - protein, 9 aa, Artificial Sequence
  Synthetic peptide derived from Homo sapiens melanoma antigens
TTINYTLWR

SEQ ID NO: 81 - protein, 9 aa, Artificial Sequence
  Synthetic peptide derived from Homo sapiens melanoma antigens
SMLEVFEGK

SEQ ID NO: 82 - protein, 8 aa, Artificial Sequence
  Synthetic peptide derived from Homo sapiens melanoma antigens
SVFAHPRK

SEQ ID NO: 83 - protein, 9 aa, Artificial Sequence
  Synthetic peptide derived from Homo sapiens melanoma antigens
IVYPPLHER

SEQ ID NO: 84 - protein, 9 aa, Artificial Sequence
  Synthetic peptide derived from Homo sapiens melanoma antigens
YVFPVIFSK

SEQ ID NO: 85 - protein, 9 aa, Artificial Sequence
  Synthetic peptide derived from Homo sapiens melanoma antigens
SVLEVFEGK

SEQ ID NO: 86 - protein, 9 aa, Artificial Sequence
  Synthetic peptide derived from Homo sapiens melanoma antigens
LVHFLLLKK

SEQ ID NO: 87 - protein, 9 aa, Artificial Sequence
  Synthetic peptide derived from Homo sapiens Her2/neu
VVFGILIKR

SEQ ID NO: 88 - protein, 9 aa, Artificial Sequence
  Synthetic peptide derived from Homo sapiens Her2/neu
KIRKYTMRR

SEQ ID NO: 89 - protein, 9 aa, Artificial Sequence
  Synthetic peptide derived from Homo sapiens Her2/neu
VLRENTSPK

SEQ ID NO: 90 - protein, 9 aa, Artificial Sequence
  Synthetic peptide derived from Homo sapiens Her2/neu
LVKSPNHVK

SEQ ID NO: 91 - protein, 9 aa, Artificial Sequence
  Synthetic peptide derived from Homo sapiens Her2/neu
KVTDFGLAR

SEQ ID NO: 92 - protein, 9 aa, Artificial Sequence
  Synthetic peptide derived from Homo sapiens Her2/neu
MALESILRR

SEQ ID NO: 93 - protein, 10 aa, Artificial Sequence
  Synthetic peptide derived from Homo sapiens Her2/neu
LVSEFSRMAR

SEQ ID NO: 94 - protein, 10 aa, Artificial Sequence
  Synthetic peptide derived from Homo sapiens Her2/neu
ASPLDSTFYR

SEQ ID NO: 95 - protein, 10 aa, Artificial Sequence
  Synthetic peptide derived from Homo sapiens p53
KTYQGSYGFK

SEQ ID NO: 96 - protein, 9 aa, Artificial Sequence
  Synthetic peptide derived from Homo sapiens p53
CTYSPALNK

SEQ ID NO: 97 - protein, 11 aa, Artificial Sequence
  Synthetic peptide derived from Homo sapiens p53
GTRVRAMAIYK

SEQ ID NO: 98 - protein, 9 aa, Artificial Sequence
  Synthetic peptide derived from Homo sapiens p53
RVRAMAIYR

SEQ ID NO: 99 - protein, 8 aa, Artificial Sequence
  Synthetic peptide derived from Homo sapiens p53
RVCACPGR

SEQ ID NO: 100 - protein, 9 aa, Artificial Sequence
  Synthetic peptide derived from Homo sapiens carcinoembryonic antigen
IYPNASLLI

SEQ ID NO: 101 - protein, 9 aa, Artificial Sequence
  Synthetic peptide derived from Homo sapiens carcinoembryonic antigen
LYGPDAPTI

SEQ ID NO: 102 - protein, 10 aa, Artificial Sequence
  Synthetic peptide derived from Homo sapiens carcinoembryonic antigen
QYSWFVNGTF

SEQ ID NO: 103 - protein, 9 aa, Artificial Sequence
  Synthetic peptide derived from Homo sapiens carcinoembryonic antigen
VYAEPPKPF

SEQ ID NO: 104 - protein, 11 aa, Artificial Sequence
  Synthetic peptide derived from Homo sapiens carcinoembryonic antigen
TYLWWVNNQSL

SEQ ID NO: 105 - protein, 10 aa, Artificial Sequence
  Synthetic peptide derived from Homo sapiens carcinoembryonic antigen
YYRPGVNLSF

SEQ ID NO: 106 - protein, 10 aa, Artificial Sequence
  Synthetic peptide derived from Homo sapiens carcinoembryonic antigen
QYSWLIDGNF

SEQ ID NO: 107 - protein, 11 aa, Artificial Sequence
  Synthetic peptide derived from Homo sapiens carcinoembryonic antigen
TYLWWVNGQSL

SEQ ID NO: 108 - protein, 9 aa, Artificial Sequence
  Synthetic peptide derived from Homo sapiens carcinoembryonic antigen
LYGPDTPII

SEQ ID NO: 109 - protein, 10 aa, Artificial Sequence
  Synthetic peptide derived from Homo sapiens carcinoembryonic antigen
SYLSGANLNF

SEQ ID NO: 110 - protein, 9 aa, Artificial Sequence
  Synthetic peptide derived from Homo sapiens carcinoembryonic antigen
TYACFVSNL

SEQ ID NO: 111 - protein, 9 aa, Artificial Sequence
  Synthetic peptide derived from Homo sapiens melanoma antigens
MYPDLESEF

SEQ ID NO: 112 - protein, 10 aa, Artificial Sequence
  Synthetic peptide derived from Homo sapiens melanoma antigens
LYILVTCLGF

SEQ ID NO: 113 - protein, 9 aa, Artificial Sequence
  Synthetic peptide derived from Homo sapiens melanoma antigens
VMPKTGLLI

SEQ ID NO: 114 - protein, 8 aa, Artificial Sequence
  Synthetic peptide derived from Homo sapiens melanoma antigens
LWGPRALI

SEQ ID NO: 115 - protein, 10 aa, Artificial Sequence
  Synthetic peptide derived from Homo sapiens melanoma antigens
SYVKVLHHTF

SEQ ID NO: 116 - protein, 9 aa, Artificial Sequence
  Synthetic peptide derived from Homo sapiens melanoma antigens
NWQYFFPVI

SEQ ID NO: 117 - protein, 10 aa, Artificial Sequence
  Synthetic peptide derived from Homo sapiens melanoma antigens
LYIFATCLGF

SEQ ID NO: 118 - protein, 9 aa, Artificial Sequence
  Synthetic peptide derived from Homo sapiens melanoma antigens
IMPKAGLLI

SEQ ID NO: 119 - protein, 10 aa, Artificial Sequence
  Synthetic peptide derived from Homo sapiens melanoma antigens
SYPPLHEWVL

SEQ ID NO: 120 - protein, 9 aa, Artificial Sequence
  Synthetic peptide derived from Homo sapiens Her2/neu
PYVSRLLGF

SEQ ID NO: 121 - protein, 9 aa, Artificial Sequence
  Synthetic peptide derived from Homo sapiens Her2/neu
SYGVTVWEF

SEQ ID NO: 122 - protein, 11 aa, Artificial Sequence
  Synthetic peptide derived from Homo sapiens Her2/neu
VYMIMVKCWMI

SEQ ID NO: 123 - protein, 9 aa, Artificial Sequence
  Synthetic peptide derived from Homo sapiens Her2/neu
RYRELVSEF

SEQ ID NO: 124 - protein, 10 aa, Artificial Sequence
  Synthetic peptide derived from Homo sapiens p53
TYQGSYGFRL

SEQ ID NO: 125 - protein, 10 aa, Artificial Sequence
  Synthetic peptide derived from Homo sapiens p53
TYQGSYGFRF

SEQ ID NO: 126 - protein, 10 aa, Artificial Sequence
  Synthetic peptide derived from Homo sapiens p53
TYSPALNKMF

SEQ ID NO: 127 - protein, 11 aa, Artificial Sequence
  Synthetic peptide derived from Homo sapiens carcinoembryonic antigen
RSDSVILNVLY

SEQ ID NO: 128 - protein, 9 aa, Artificial Sequence
  Synthetic peptide derived from Homo sapiens carcinoembryonic antigen
ITDNNSGSY

SEQ ID NO: 129 - protein, 11 aa, Artificial Sequence
  Synthetic peptide derived from Homo sapiens carcinoembryonic antigen
HSDPVILNVLY

SEQ ID NO: 130 - protein, 10 aa, Artificial Sequence
  Synthetic peptide derived from Homo sapiens carcinoembryonic antigen
PTDSPSYTYY

SEQ ID NO: 131 - protein, 9 aa, Artificial Sequence
  Synthetic peptide derived from Homo sapiens carcinoembryonic antigen
AADNPPAQY

SEQ ID NO: 132 - protein, 9 aa, Artificial Sequence
  Synthetic peptide derived from Homo sapiens carcinoembryonic antigen
ITDKNSGLY

SEQ ID NO: 133 - protein, 11 aa, Artificial Sequence
  Synthetic peptide derived from Homo sapiens carcinoembryonic antigen
RSDPVTLDVLY

SEQ ID NO: 134 - protein, 10 aa, Artificial Sequence
  Synthetic peptide derived from Homo sapiens carcinoembryonic antigen
HSASNPSPQY

SEQ ID NO: 135 - protein, 9 aa, Artificial Sequence
  Synthetic peptide derived from Homo sapiens Her2/neu
VMDGVGSPY

SEQ ID NO: 136 - protein, 10 aa, Artificial Sequence
  Synthetic peptide derived from Homo sapiens Her2/neu
CTQIAKGMSY

SEQ ID NO: 137 - protein, 9 aa, Artificial Sequence
  Synthetic peptide derived from Homo sapiens Her2/neu
LLDIDETEY

SEQ ID NO: 138 - protein, 10 aa, Artificial Sequence
  Synthetic peptide derived from Homo sapiens Her2/neu
FTHQSDVWSY

SEQ ID NO: 139 - protein, 10 aa, Artificial Sequence
  Synthetic peptide derived from Homo sapiens Her2/neu
PADPLDSTFY

SEQ ID NO: 140 - protein, 10 aa, Artificial Sequence
  Synthetic peptide derived from Homo sapiens Her2/neu
MTDLVDAEEY

SEQ ID NO: 141 - protein, 9 aa, Artificial Sequence
  Synthetic peptide derived from Homo sapiens Her2/neu
LTDSPQPEY

SEQ ID NO: 142 - protein, 10 aa, Artificial Sequence
  Synthetic peptide derived from Homo sapiens Her2/neu
FSPAFDNLYY

SEQ ID NO: 143 - protein, 10 aa, Artificial Sequence
  Synthetic peptide derived from Homo sapiens Her2/neu
GTDTAENPEY

SEQ ID NO: 144 - protein, 10 aa, Artificial Sequence
  Synthetic peptide derived from Homo sapiens melanoma antigens
ASDFSTTINY

SEQ ID NO: 145 - protein, 8 aa, Artificial Sequence
  Synthetic peptide derived from Homo sapiens melanoma antigens
VTDLGLSY

SEQ ID NO: 146 - protein, 9 aa, Artificial Sequence
  Synthetic peptide derived from Homo sapiens melanoma antigens
MQDLVQENY

SEQ ID NO: 147 - protein, 10 aa, Artificial Sequence
  Synthetic peptide derived from Homo sapiens melanoma antigens
ASSLPTTMNY

SEQ ID NO: 148 - protein, 9 aa, Artificial Sequence
  Synthetic peptide derived from Homo sapiens melanoma antigens
GTVVGNWQY

SEQ ID NO: 149 - protein, 9 aa, Artificial Sequence
  Synthetic peptide derived from Homo sapiens melanoma antigens
EVDPIGHLY

SEQ ID NO: 150 - protein, 10 aa, Artificial Sequence
  Synthetic peptide derived from Homo sapiens melanoma antigens
LTDHFVQENY

SEQ ID NO: 151 - protein, 9 aa, Artificial Sequence
  Synthetic peptide derived from Homo sapiens melanoma antigens
ITGGPHISY

SEQ ID NO: 152 - protein, 10 aa, Artificial Sequence
  Synthetic peptide derived from Homo sapiens p53
PTQKTYQGSY

SEQ ID NO: 153 - protein, 10 aa, Artificial Sequence
  Synthetic peptide derived from Homo sapiens p53
GTDKSVTCTY

SEQ ID NO: 154 - protein, 10 aa, Artificial Sequence
  Synthetic peptide derived from Homo sapiens p53
RVDGNLRVEY

SEQ ID NO: 155 - protein, 11 aa, Artificial Sequence
  Synthetic peptide derived from Homo sapiens p53
GSDCTTIHYNY

SEQ ID NO: 156 - protein, 325 aa, Unknown
  Polypeptide 1
MGMQVQIQSLFLLLLWVPGSRGVPISHLDILKKLSEYLQLVGAAAISPSYTYYRKAAATYACFVSNLKVTDFGLARGAAAHLFGYSIYKNAQYSWFVNGTFKAAAKVFGSLAFVNAAAPYVSRLLGINIMIGHLVGVNLLTFWNPPVIVYPPLHERNAAAEYLQLMFGINAIMPKAGLLINKTYQGSYGFKKAAARVRAMAIYRNAAARYARDPQRFGAAAKLCPVQLWVNASMPPPGTRVGAAAVVLGVVFGIAKFVAAWTLKAAAKVAEIVHFLNTYSPALNKMFKAASYGFRLGFFKAAASSFSTTINKKAAAVVFGILIKR

SEQ ID NO: 157 - protein, 326 aa, Unknown
  Polypeptide 2
MGMQVQIQSLFLLLLWVPGSRGIVYPPLHERNAAAEYLQLLFGINAIMPKAGLLINKTYQGSYGFKKAAARVRAMAIYRNAAARYARDPQRFGAAAKLCPVQLWVNASMPPPGTRVGAAAVVLGVVFGIAKFVAAWTLKAAAKVAELVWFLNAAATYSPALNKMFKAASYGFRLGFFKAAASSFSTTINKKAAAVVFGILIKRVPISHLGILKKLSEYLQLVGAAAISPSYTYYRKAAATYACFVSNLKVFGSLAFVNAAAPYVSRLLGINAHLFGYSDYKNAQYSWFVNGTFKAAAKVTDFGLARNIMMGHLVGVNLLTFWNPPV

SEQ ID NO: 158 - protein, 331 aa, Unknown
  Polypeptide 3
MGMQVQIQSLFLLLLWVPGSRGTYSPALNKMFKAASYGFRLGFFKAAASSFSTTINKKAAAVVFGILIKRNAAAAKFVAAWTLKAAAKVAEIVHFLKVTDFGLARGAAAHLFPYSWYKNATYACFVSNLKAAAVPISHLEILKKLSEYLQLVGAAAISPSYTYYRKAAAQYSWFVNGTFKAAAKVFGSLAFVNAAAPYVSRLLGINIMIGHLVGVNLLTFWNPPVIVYPPLHERNAAAEYLQLMFGINSMPPPGTRVGAAAVVLGVVFGINAIMPKAGLLINKTYQGSYGFKKAAARVRAMAIYRNAAARYARDPQRFGAAAKLCPVQLWV

SEQ ID NO: 159 - DNA, 978 bp, Unknown
  Polynucleotide 1
atgggaatgcaggtgcaaatacagtctctcttccttttgcttctctgggttccaggatcacggggcgtccccatttcccatctcgatattctgaagaagctgagcgagtacctgcaactggtcggcgctgcagctattagccctagctacacttattatcggaaggctgctgctacctatgcctgtttcgtgtctaatctcaaagtcacagacttcgggctcgcaagaggggctgccgctcacctgttcgggtactctatctataaaaacgcccaatattcctggtttgtgaatggaaccttcaaagctgcagccaaggtcttcggcagcctggcatttgtcaacgccgctgctccctacgtgagccggctcctcgggattaatattatgatcggccacctggtgggagtgaatctgctcacattttggaaccctccagtgatcgtgtacccacctctccatgaaaggaacgccgcagccgaatatctgcagctgatgttcggcatcaatgccattatgcctaaagccggactgctgatcaacaagacttaccagggctcttacggcttcaagaaggctgcagcccgcgtcagagccatggctatctaccgcaacgccgccgctcggtacgccagggacccccagcgctttggggctgccgccaagctgtgcccagtgcagctgtgggtgaacgcttctatgccccctccaggcacaagagtgggagccgctgctgtcgtgctgggagtcgtgttcggcatcgcaaagtttgtggccgcctggaccctcaaggcagcagcaaaagtcgcagagattgtgcactttctgaacacttactcccccgcactgaacaaaatgtttaaagccgcatcctatggcttcaggctggggttctttaaggccgccgcaagctccttctctaccacaatcaataagaaggccgctgccgtggtgttcggaatcctcatcaaaagatag

SEQ ID NO: 160 - DNA, 981 bp, Unknown
  Polynucleotide 2
atgggaatgcaggtgcaaatacagtctctcttccttttgcttctctgggttccaggatcacggggcattgtgtacccccccctgcacgagcggaacgctgctgcagaatatctccagctcctgttcggcattaacgccattatgcctaaagcaggcctgctcatcaacaaaacttaccagggaagctatgggttcaagaaggcagctgcaagagtcagggccatggccatctatcggaatgctgcagcacgctatgccagggatcctcaaaggtttggggccgccgccaagctctgtcccgtgcaactctgggtcaatgcctccatgcctccacccggaacaagagtcggagccgccgccgtggtcctcggggtggtcttcgggatcgccaaattcgtcgccgcctggacactgaaggccgctgctaaggtcgccgaactggtgtggttcctgaacgctgccgcaacatactcccctgctctcaacaaaatgtttaaggctgcctcttacggctttagactgggatttttcaaggcagctgcctctagcttctctacaactatcaataaaaaggccgcagccgtcgtgttcgggatcctgatcaaacgggtgccaatcagccatctcggcatcctgaagaaactgtctgagtacctgcagctggtgggggctgccgctatctctccaagctacacctactatagaaaggcagctgctacctacgcttgcttcgtgagcaatctgaaagtgtttggctccctggcattcgtcaacgcagctgccccatacgtgtcccgcctcctgggaattaacgctcacctgtttggatatagcgactataagaatgcccagtactcctggttcgtgaacggcaccttcaaggcagccgcaaaggtgaccgactttggcctggctcgcaacattatgatgggccatctggtgggcgtgaatctcctgactttttggaatccccctgtgtag

SEQ ID NO: 161 - DNA, 996 bp, Unknown
  Polynucleotide 3
atgggaatgcaggtgcaaatacagtctctcttccttttgcttctctgggttccaggatcacggggcacctactctcctgcactcaataaaatgtttaaggccgcatcctacggcttcaggctcggattctttaaggccgccgcaagcagcttttctactacaatcaacaagaaagctgccgcagtggtctttgggatcctcatcaaaaggaacgcagccgcagctaagttcgtcgctgcttggaccctgaaagccgccgccaaagtcgctgaaatcgtccatttcctcaaggtgacagattttggactggctagaggcgccgccgctcacctgttcccttattcctggtacaaaaacgccacctacgcttgtttcgtgagcaacctgaaggctgccgcagtgccaatctcccatctcgagatcctgaagaaactgtctgagtacctgcagctcgtcggcgccgccgcaatttctccctcttacacttactatcgcaaagctgccgctcaatacagctggtttgtgaacggaactttcaaggctgccgctaaggtgttcggatccctggctttcgtgaatgccgccgccccctatgtgagccggctgctgggaattaatattatgattggccacctggtcggagtgaacctgctgacattctggaatcctcctgtgattgtctacccacctctgcacgaaagaaacgccgccgccgagtatctccagctcatgtttgggatcaatagcatgccaccccccggcaccagagtgggggcagcagccgtcgtcctgggcgtggtgttcgggatcaacgcaatcatgccaaaggccgggctgctgattaacaagacataccaggggtcctatggctttaagaaggccgcagctcgcgtgcgggctatggctatctataggaatgcagccgctagatatgctcgcgacccacagcggttcggcgcagctgcaaagctgtgccccgtgcaactctgggtgtag

SEQ ID NO: 162 - protein, 9 aa, Artificial Sequence
  Synthetic peptide derived from Homo sapiens carcinoembryonic antigen
IMIGHLVGV

SEQ ID NO: 163 - protein, 9 aa, Artificial Sequence
  Synthetic peptide derived from Homo sapiens carcinoembryonic antigen
LLTFWNPPV

SEQ ID NO: 164 - protein, 9 aa, Artificial Sequence
  Synthetic peptide derived from Homo sapiens Her2
KVFGSLAFV

SEQ ID NO: 165 - protein, 9 aa, Artificial Sequence
  Synthetic peptide derived from Homo sapiens Her2
VVLGVVFGI

SEQ ID NO: 166 - protein, 9 aa, Artificial Sequence
  Synthetic peptide derived from Homo sapiens p53
SMPPPGTRV

SEQ ID NO: 167 - protein, 9 aa, Artificial Sequence
  Synthetic peptide derived from Homo sapiens p53
KLCPVQLWV

SEQ ID NO: 168 - protein, 9 aa, Artificial Sequence
  Synthetic peptide derived from Homo sapiens melanoma antigens
KVAEIVHFL

SEQ ID NO: 169 - protein, 9 aa, Artificial Sequence
  Synthetic peptide derived from Homo sapiens melanoma antigens
KLSEYLQLV

SEQ ID NO: 170 - protein, 9 aa, Artificial Sequence
  Synthetic peptide derived from Homo sapiens carcinoembryonic antigen
HLFGYSIYK

SEQ ID NO: 171 - protein, 9 aa, Artificial Sequence
  Synthetic peptide derived from Homo sapiens carcinoembryonic antigen
ISPSYTYYR

SEQ ID NO: 172 - protein, 9 aa, Artificial Sequence
  Synthetic peptide derived from Homo sapiens Her2
KVTDFGLAR

SEQ ID NO: 173 - protein, 9 aa, Artificial Sequence
  Synthetic peptide derived from Homo sapiens Her2
VVFGILIKR

SEQ ID NO: 174 - protein, 9 aa, Artificial Sequence
  Synthetic peptide derived from Homo sapiens melanoma antigens
SSFSTTINK

SEQ ID NO: 175 - protein, 9 aa, Artificial Sequence
  Synthetic peptide derived from Homo sapiens melanoma antigens
IVYPPLHER

SEQ ID NO: 176 - protein, 10 aa, Artificial Sequence
  Synthetic peptide derived from Homo sapiens p53
KTYQGSYGFK

SEQ ID NO: 177 - protein, 9 aa, Artificial Sequence
  Synthetic peptide derived from Homo sapiens p53
RVRAMAIYR

SEQ ID NO: 178 - protein, 10 aa, Artificial Sequence
  Synthetic peptide derived from Homo sapiens carcinoembryonic antigen
QYSWFVNGTF

SEQ ID NO: 179 - protein, 9 aa, Artificial Sequence
  Synthetic peptide derived from Homo sapiens carcinoembryonic antigen
TYACFVSNL

SEQ ID NO: 180 - protein, 9 aa, Artificial Sequence
  Synthetic peptide derived from Homo sapiens Her2
PYVSRLLGI

SEQ ID NO: 181 - protein, 9 aa, Artificial Sequence
  Synthetic peptide derived from Homo sapiens Her2
RYARDPQRF

SEQ ID NO: 182 - protein, 9 aa, Artificial Sequence
  Synthetic peptide derived from Homo sapiens melanoma antigens
EYLQLMFGI

SEQ ID NO: 183 - protein, 9 aa, Artificial Sequence
  Synthetic peptide derived from Homo sapiens melanoma antigens
IMPKAGLLI

SEQ ID NO: 184 - protein, 9 aa, Artificial Sequence
  Synthetic peptide derived from Homo sapiens p53
SYGFRLGFF

SEQ ID NO: 185 - protein, 10 aa, Artificial Sequence
  Synthetic peptide derived from Homo sapiens p53
TYSPALNKMF

SEQ ID NO: 186 - protein, 9 aa, Artificial Sequence
  Synthetic peptide derived from Homo sapiens melanoma antigens
VPISHLDIL

SEQ ID NO: 187 - protein, 9 aa, Artificial Sequence
  Synthetic peptide derived from Homo sapiens carcinoembryonic antigen
IMMGVLVGV

SEQ ID NO: 188 - protein, 9 aa, Artificial Sequence
  Synthetic peptide derived from Homo sapiens melanoma antigens
KVAELVWFL

SEQ ID NO: 189 - protein, 9 aa, Artificial Sequence
  Synthetic peptide derived from Homo sapiens carcinoembryonic antigen
HLFGYSDYK

SEQ ID NO: 190 - protein, 9 aa, Artificial Sequence
  Synthetic peptide derived from Homo sapiens melanoma antigens
EYLQLLFGI

SEQ ID NO: 191 - protein, 9 aa, Artificial Sequence
  Synthetic peptide derived from Homo sapiens melanoma antigens
VPISHLGIL

SEQ ID NO: 192 - protein, 9 aa, Artificial Sequence
  Synthetic peptide derived from Homo sapiens carcinoembryonic antigen
IMIGHLVGV

SEQ ID NO: 193 - protein, 9 aa, Artificial Sequence
  Synthetic peptide derived from Homo sapiens melanoma antigens
KVAEIVHFL

SEQ ID NO: 194 - protein, 9 aa, Artificial Sequence
  Synthetic peptide derived from Homo sapiens carcinoembryonic antigen
HLFPYSWYK

SEQ ID NO: 195 - protein, 9 aa, Artificial Sequence
  Synthetic peptide derived from Homo sapiens melanoma antigens
EYLQLMFGI

SEQ ID NO: 196 - protein, 9 aa, Artificial Sequence
  Synthetic peptide derived from Homo sapiens melanoma antigens
VPISHLEIL